(19) United States
(12) Patent Application Publication
(10) Pub. No.: US 2025/0014755 A1
(43) Pub. Date: Jan. 9, 2025

(54) SYSTEMS AND METHODS FOR DISEASE ASSESSMENTS

(71) Applicant: Synergenz Bioscience Limited, Hong Kong (CN)

(72) Inventor: Robert Peter Young, Auckland (NZ)

(73) Assignee: Synergenz Bioscience Limited, Hong Kong (CN)

(21) Appl. No.: 18/709,935

(22) PCT Filed: Dec. 9, 2022
(86) PCT No.: PCT/NZ2022/050165

§ 371 Date: May 14, 2024

(30) Foreign Application Priority Data
Dec. 9, 2021 (NZ) 783327
Jun. 7, 2022 (NZ) 789164

(51) Int. Cl. G16H 50/30; G16B 20/20; G16H 10/40

(52) U.S. Cl. CPC G16H 50/30; G16B 20/20; G16H 10/40

(57) ABSTRACT

The present disclosure provides systems and methods for disease assessments. In some examples, the present disclosure provides a method for processing or analyzing a bodily sample of a subject. The method may comprise analyzing said bodily sample to yield a data set comprising at least one genetic polymorphism in the bodily sample. The at least one genetic polymorphism may be associated with a pulmonary or respiratory disease or disorder. The method may further comprise computer processing the data set and a clinical health parameter of the subject to determine a risk-value of the pulmonary or respiratory disease or disorder in the subject. The method may further comprise electronically outputting a report that identifies the risk-value of the pulmonary or respiratory disease or disorder in the subject.

### CROSS-REFERENCE TO RELATED APPLICATION

This application claims the benefit of New Zealand Provisional Patent Application No. 783327 filed on Dec. 9, 2021, and New Zealand Provisional Patent Application No. 789164 filed on Jun. 7, 2022, which are incorporated by reference herein in their entirety.

### BACKGROUND

Lung cancer is one of the most common and deadly cancers. The causes of lung cancer have been attributed to various factors, such as cigarette smoking, occupational exposure, genetic factors, radon exposure, exposure to other acro-pollutants, or dietary factors. Lung cancer-associated risks can be determined for a subject. For example, a subject's lifestyle (e.g., cigarette smoking habit) can increase the subject's risk of developing lung cancer. Additionally, there are genetic markers such as genetic variants or polymorphisms that are associated with the risks of developing cancer.

### SUMMARY

Recognized herein is an industry-wide need for improved screening and diagnosis methods for cancers, such as lung cancer. Further recognized herein is an industry-wide need for analysis of genetic markers with reduced false positive or false negative results. The present disclosure describes methods for determining a the risk of developing a pulmonary disease or disorder based on analyzing genetic markers. Also described herein are improved methods for determining a lung cancer-associated risk. Methods as described herein result in increased sensitivity or increased specificity compared to other methods available. Methods as described herein may be advantageous in diagnosing a subject early for early treatment options or in determining a therapeutic regiment for the subject.

An aspect of the present disclosure provides a method for processing or analyzing a bodily sample in a subject, comprising: analyzing said bodily sample to yield a first data set comprising at least one genetic polymorphism in said bodily sample, wherein said at least one genetic polymorphism is associated with a first pulmonary or respiratory disease or disorder: analyzing said bodily sample to yield a second data set comprising at least one genetic polymorphism in said bodily sample, wherein said at least one genetic polymorphism is associated with a second pulmonary or respiratory disease or disorder: imaging a pulmonary nodule from said subject to determine at least one characteristic associated with said image of said pulmonary nodule: obtaining data with respect to at least one biomarker of said subject: computer processing said first data set and said second data set to yield a third data set, and analyzing said third data set in conjunction with said at least one characteristic of said pulmonary nodule and said at least one biomarker to determine the risk associated with said pulmonary nodule; and electronically outputting a report that identifies the risk presented by said pulmonary nodule. In some embodiments, said pulmonary nodule is detected during a procedure or visit that is related to lung cancer screening. In some embodiments, said pulmonary nodule is detected during a procedure or visit that is not related to lung cancer screening. In some embodiments, said pulmonary nodule is an intermediate risk nodule. In some embodiments, said pulmonary nodule is an indeterminate bodily sample. In some embodiments, said first pulmonary or respiratory disease or disorder or said second pulmonary or respiratory disease or disorder is lung cancer. In some embodiments, said first pulmonary or respiratory disease or disorder or said second pulmonary or respiratory disease or disorder is airflow limitation or chronic obstructive pulmonary disease (COPD). In some embodiments, the method further comprises determining the level of said subject's risk of developing lung cancer, and categorizing said risk as high, intermediate, or low. In some embodiments, the method further comprises gathering data on one or more subjects in an intermediate risk group. In some embodiments, the method further comprises gathering data on one or more subjects in an intermediate risk group who move to a low-risk group. In some embodiments, the method further comprises gathering data on one or more subjects in an intermediate risk group who move to a higher-risk group. In some embodiments, the method further comprises comparing said risk associated with said pulmonary nodule with said data from said one or more subjects in an intermediate risk group. In some embodiments, said risk presented by said pulmonary nodule is determined at an Area Under Curve (AUC) at least 1.01-, 1.02-, 1.03-, 1.04-, 1.05-, or 1.06-fold greater than a corresponding AUC achieved with a clinical score. In some embodiments, said risk presented by said pulmonary nodule is determined at a sensitivity at least 1.01-, 1.02-, 1.03-, 1.04-, 1.05-, or 1.06-fold greater than a corresponding sensitivity achieved with said clinical score. In some embodiments, said risk presented by said pulmonary nodule is determined at a specificity at least 1.01-, 1.02-, 1.03-, 1.04-, 1.05-, or 1.06-fold greater than a corresponding specificity achieved with said clinical score. In some embodiments, said risk presented by said pulmonary nodule is determined at: a sensitivity at least 1.01-, 1.02-, 1.03-, 1.04-, 1.05-, or 1.06-fold greater than a corresponding sensitivity achieved with said clinical score, and a specificity no less than a corresponding specificity achieved with said clinical score. In some embodiments, said risk presented by said pulmonary nodule is determined at: a sensitivity no less than a corresponding sensitivity achieved with said clinical score, and a specificity at least 1.01-, 1.02-, 1.03-, 1.04-, 1.05-, or 1.06-fold greater than a corresponding specificity achieved with said clinical score. In some embodiments, said risk presented by said pulmonary nodule is determined at: a sensitivity at least 1.01-, 1.02-, 1.03-, 1.04-, 1.05-, or 1.06-fold greater than a corresponding sensitivity achieved with said clinical score, and a specificity at least 1.01-, 1.02-, 1.03-, 1.04-, 1.05-, or 1.06-fold greater than a corresponding specificity achieved with said clinical score. In some embodiments, said risk presented by said pulmonary nodule indicates a presence, a reduced risk, or an elevated risk of said pulmonary or respiratory disease or disorder. In some embodiments, the method further comprises analyzing at least one of: total number of pulmonary nodule, pulmonary nodule size, pulmonary nodule speculation, pulmonary nodule type, pulmonary nodule location, and pulmonary nodule lobulation. In some embodiments, the method further comprises determining said clinical score. In some embodiments, said first data set or said second data set comprises at least two, at least five, at least ten, or at least fifteen single nucleotide polymorphisms (SNPs) selected from: (1) a polymorphism at rs1489759 of Hedgehog Interacting Protein (HHIP) gene, (2) a polymorphism at rs13141641 of Hedgehog Interacting Protein (HHIP) gene, (3) a polymorphism at rs2202507 of Glycophorin A (GYPA) gene, (4) a polymorphism at rs7671167 of Family with Sequence Similarity 13 Member A (FAM13A) gene, (5) a polymorphism at rs754388 of Ras and Rab Interactor 3 (RIN3) gene, (6) a polymorphism at rs11168048 of 5-Hydroxytryptamine Receptor 4 (HTR4) gene, (7) a polymorphism at rs58863591 of Deleted in Liver Cancer 1 (DLC1) gene, (8) a polymorphism at rs1422795 of A Disintegrin and Metalloproteinase 19 (ADAM19) gene, (9) a polymorphism at rs2306022 of Integrin Alpha 11 (ITGA11) gene, (10) a polymorphism at rs2808630 of C-Reactive Protein (CRP) gene, (11) a polymorphism at rs3087386 of DNA Repair Protein (Rev1) gene, (12) a polymorphism at rs645419 of Matrix Metalloproteinase-12 (MMP12) gene, (13) a polymorphism at rs763110 of Fas Ligand (FasL) gene. (14) a polymorphism at rs1052486 of HLA-B-Associated Transcript 3 (BAT3) gene, (15) a polymorphism at rs1800797 of Interleukin-6 (IL-6) gene, (16) a polymorphism at rs2070600 of Advanced Glycosylation End Product-Specific Receptor (AGER) gene, (17) a polymorphism at rs2656069 of Iron Regulatory Protein (IREB) gene, (18) a polymorphism at rs16969968 of Cholinergic Receptor Nicotinic Alpha 3/5 (CHRNA3/5) gene, (19) a polymorphism at rs402710 of Telomerase Reverse Transcriptase (TERT) gene, and (20) a polymorphism at rs7937 of Cytochrome P450 Family 2 Subfamily A Member 6 (CYP2A6) gene. In some embodiments, said first data set or said second data set comprises any SNP in full linkage disequilibrium to one or more SNPs as disclosed herein.

Another aspect of the present disclosure provides a method for processing or analyzing a bodily sample of a subject, comprising: analyzing said bodily sample to yield a first data set comprising at least one genetic polymorphism in said bodily sample, wherein said at least one genetic polymorphism is associated with a first pulmonary or respiratory disease or disorder, and a second data set comprising at least one genetic polymorphism in said bodily sample, wherein said at least one genetic polymorphism is associated with a second pulmonary or respiratory disease or disorder: imaging a pulmonary nodule from said subject to determine at least one characteristic associated with said image of said pulmonary nodule: obtaining data with respect to at least one biomarker of said subject computer processing said first data set and said second data set to produce a third data set, and further analyzing said third data set and said at least one characteristic of said pulmonary nodule, to determine the risk associated with said pulmonary nodule to an accuracy of at least about 80%, wherein said accuracy is a percentage of a plurality of independent test samples corresponding to (1) a first plurality of subjects having a pulmonary nodule exhibiting malignancy to (2) a second plurality of subjects having a pulmonary nodule not exhibiting malignancy; and electronically outputting a report that identifies said risk presented by said pulmonary nodule. In some embodiments, said pulmonary nodule is detected during a procedure or visit that is related to lung cancer screening. In some embodiments, said pulmonary nodule is detected during a procedure or visit that is not related to lung cancer screening. In some embodiments, said pulmonary nodule is an intermediate risk nodule. In some embodiments, said pulmonary nodule is an indeterminate bodily sample. In some embodiments, the method further comprises determining the level of said subject's risk of developing lung cancer, and categorizing said risk as high, intermediate, or low. In some embodiments, said first pulmonary or respiratory disease or disorder or said second pulmonary or respiratory disease or disorder is lung cancer. In some embodiments, said first pulmonary or respiratory disease or disorder or said second pulmonary or respiratory disease or disorder is airflow limitation or chronic obstructive pulmonary disease (COPD). In some embodiments, the method further comprises gathering data on one or more subjects in an intermediate risk group. In some embodiments, the method further comprises gathering data on one or more subjects in an intermediate risk group who move to a low-risk group. In some embodiments, the method further comprises gathering data on one or more subjects in an intermediate risk group who move to a higher-risk group. In some embodiments, the method further comprises comparing said risk associated with said pulmonary nodule with said data from said one or more subjects in an intermediate risk group. In some embodiments, said risk associated with said pulmonary nodule is determined at an accuracy of at least about 60%. In some embodiments, said risk associated with said pulmonary nodule is determined at an accuracy of at least about 70%. In some embodiments, said accuracy comprises a ratio of (1) a first sum of true positive cases and true negative cases, to (2) a second sum of true positive cases, true negative cases, false positive cases, and false negative cases. In some embodiments, said risk associated with said pulmonary nodule is determined at a sensitivity of at least about 80%. In some embodiments, said risk associated with said pulmonary nodule is determined at a sensitivity of at least about 90%. In some embodiments, said risk associated with said pulmonary nodule is determined at a sensitivity of at least about 95%. In some embodiments, said sensitivity comprises a ratio of (1) a number of true positive cases, to (2) a sum of true positive cases and false negative cases. In some embodiments, said sensitivity is a percentage of a plurality of independent test samples corresponding to (1) a first plurality of subjects receiving an elevated risk-value of said pulmonary or respiratory disease or disorder, relative to (2) a second plurality of subjects having said pulmonary or respiratory disease or disorder that is correctly determined to have or be at risk of said pulmonary or respiratory disease or disorder. In some embodiments, said risk associated with said pulmonary nodule is determined at a specificity of at least about 80%. In some embodiments, said risk associated with said pulmonary nodule is determined at a specificity of at least about 90%. In some embodiments, said risk associated with said pulmonary nodule is determined at a specificity of at least about 95%. In some embodiments, said specificity comprises a ratio of (1) a number of true negative cases, to (2) a sum of true negative cases and false positive cases. In some embodiments, said specificity is a percentage of a plurality of independent test samples corresponding to (1) a first plurality of subjects receiving an non-elevated risk associated with said nodule, relative to (2) a second plurality who do not have negative clinical indications associated with pulmonary nodule, that is correctly determined to not have or not be at risk of said pulmonary or respiratory disease or disorder. In some embodiments, said risk associated with said pulmonary nodule determined at an Area Under Curve (AUC) of at least about 80%. In some embodiments, said risk associated with said pulmonary nodule determined at an AUC of at least about 90%. In some embodiments, said risk associated with said pulmonary nodule is determined at an AUC of at least about 95%. In some embodiments, said first data set or said second data set comprises at least two, at least five, at least ten, or at least fifteen single nucleotide polymorphisms (SNPs) selected from: (1) a polymorphism at rs1489759 of Hedgehog Interacting Protein (HHIP) gene, (2) a polymorphism at rs13141641 of Hedgehog Interacting Protein (HHIP) gene, (3) a polymorphism at rs2202507 of Glycophorin A (GYPA) gene, (4) a polymorphism at rs7671167 of Family with Sequence Similarity 13 Member A (FAM13A) gene, (5) a polymorphism at rs754388 of Ras and Rab Interactor 3 (RIN3) gene, (6) a polymorphism at rs11168048 of 5-Hydroxytryptamine Receptor 4 (HTR4) gene, (7) a polymorphism at rs58863591 of Deleted in Liver Cancer 1 (DLC1) gene, (8) a polymorphism at rs1422795 of A Disintegrin and Metalloproteinase 19 (ADAM19) gene. (9) a polymorphism at rs2306022 of Integrin Alpha 11 (ITGA11) gene, (10) a polymorphism at rs2808630 of C-Reactive Protein (CRP) gene, (11) a polymorphism at rs3087386 of DNA Repair Protein (Rev1) gene, (12) a polymorphism at rs645419 of Matrix Metalloproteinase-12 (MMP12) gene, (13) a polymorphism at rs763110 of Fas Ligand (FasL) gene, (14) a polymorphism at rs1052486 of HLA-B-Associated Transcript 3 (BAT3) gene, (15) a polymorphism at rs1800797 of Interleukin-6 (IL-6) gene, (16) a polymorphism at rs2070600 of Advanced Glycosylation End Product-Specific Receptor (AGER) gene, (17) a polymorphism at rs2656069 of Iron Regulatory Protein (IREB) gene, (18) a polymorphism at rs16969968 of Cholinergic Receptor Nicotinic Alpha 3/5 (CHRNA3/5) gene, (19) a polymorphism at rs402710 of Telomerase Reverse Transcriptase (TERT) gene, and (20) a polymorphism at rs7937 of Cytochrome P450 Family 2 Subfamily A Member 6 (CYP2A6) gene. In some embodiments, said first data set or said second data set comprises any SNP in full linkage disequilibrium to one or more of said SNPs as disclosed herein. In some embodiments, said risk associated with said pulmonary nodule indicates a presence, a reduced risk, or an elevated risk of said pulmonary or respiratory disease or disorder.

Another aspect of the present disclosure provides a method for processing or analyzing a bodily sample of a subject, comprising: (a) analyzing said bodily sample to yield a first data set comprising at least one genetic polymorphism in said bodily sample, wherein said at least one genetic polymorphism is associated with a first pulmonary or respiratory disease or disorder, and a second data set comprising at least one genetic polymorphism in said bodily sample, wherein said at least one genetic polymorphism is associated with a second pulmonary or respiratory disease or disorder, and wherein said first data set or said second data set comprises at least two, at least five, at least ten, or at least fifteen single nucleotide polymorphisms (SNPs) selected from: (1) a polymorphism at rs1489759 of Hedgehog Interacting Protein (HHIP) gene, (2) a polymorphism at rs13141641 of Hedgehog Interacting Protein (HHIP) gene, (3) a polymorphism at rs2202507 of Glycophorin A (GYPA) gene, (4) a polymorphism at rs7671167 of Family with Sequence Similarity 13 Member A (FAM13A) gene, (5) a polymorphism at rs754388 of Ras and Rab Interactor 3 (RIN3) gene, (6) a polymorphism at rs11168048 of 5-Hydroxytryptamine Receptor 4 (HTR4) gene, (7) a polymorphism at rs58863591 of Deleted in Liver Cancer 1 (DLC1) gene, (8) a polymorphism at rs1422795 of A Disintegrin and Metalloproteinase 19 (ADAM19) gene, (9) a polymorphism at rs2306022 of Integrin Alpha 11 (ITGA11) gene, (10) a polymorphism at rs2808630 of C-Reactive Protein (CRP) gene, (11) a polymorphism at rs3087386 of DNA Repair Protein (Rev1) gene, (12) a polymorphism at rs645419 of Matrix Metalloproteinase-12 (MMP12) gene, (13) a polymorphism at rs763110 of Fas Ligand (FasL) gene, (14) a polymorphism at rs1052486 of HLA-B-Associated Transcript 3 (BAT3) gene, (15) a polymorphism at rs1800797 of Interleukin-6 (IL-6) gene, (16) a polymorphism at rs2070600 of Advanced Glycosylation End Product-Specific Receptor (AGER) gene, (17) a polymorphism at rs2656069 of Iron Regulatory Protein (IREB) gene, (18) a polymorphism at rs16969968 of Cholinergic Receptor Nicotinic Alpha 3/5 (CHRNA3/5) gene, (19) a polymorphism at rs402710 of Telomerase Reverse Transcriptase (TERT) gene, and (20) a polymorphism at rs7937 of Cytochrome P450 Family 2 Subfamily A Member 6 (CYP2A6) gene; (b) imaging a pulmonary nodule from said subject to determine at least one characteristic associated with said image of said pulmonary nodule; and (c) computer processing said first data set and said second data set to yield a third data set, and analyzing said third data set in conjunction with said at least one characteristic of said pulmonary nodule of (b) to determine the risk associated with said pulmonary nodule. In some embodiments, said pulmonary nodule is detected during a procedure or visit that is related to lung cancer screening. In some embodiments, said pulmonary nodule is detected during a procedure or visit that is not related to lung cancer screening. In some embodiments, said pulmonary nodule is an intermediate risk nodule. In some embodiments, said pulmonary nodule is an indeterminate bodily sample. In some embodiments, said first pulmonary or respiratory disease or disorder or said second pulmonary or respiratory disease or disorder is lung cancer. In some embodiments, said first pulmonary or respiratory disease or disorder or said second pulmonary or respiratory disease or disorder is airflow limitation or chronic obstructive pulmonary disease (COPD). In some embodiments, said first data or said second data set further comprises at least one SNP in full linkage disequilibrium to one or more of said SNPs. In some embodiments, said first data set or said second data set comprises 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, or all 22 single nucleotide polymorphisms (SNPs) selected from: (1) a polymorphism (e.g., GG genotype) at rs1489759 of human Hedgehog interacting protein (HHIP) gene, (2) a polymorphism (e.g., CC genotype) at rs13141641 of human Hedgehog interacting protein (HHIP) gene, (3) a polymorphism (e.g., GG or/and CC genotype) at rs2202507 of glycophorin A (GYPA) gene, (4) a polymorphism (e.g., CC genotype) at rs7671167 of family with sequence similarity 13 member A (FAM13A) gene, (5) a polymorphism (e.g., CG or GG genotype) at rs754388 of Ras and Rab interactor 3 (RIN3) gene, (6) a polymorphism (e.g., CC genotype) at rs11168048 of 5-hydroxytryptamine receptor 4 (HTR4) gene, (7) a polymorphism (e.g., CT, or TT genotype) at rs58863591 of deleted in liver cancer 1 (DLC1) gene, (8) a polymorphism (e.g., CC genotype) at rs1422795 in a disintegrin and metalloproteinase 19 (ADAM19) gene, (9) a polymorphism (e.g., TT or TC genotype) at rs2306022 integrin alpha 11 (ITGA11) gene, (10) a polymorphism (e.g., CC genotype) at rs2808630 of C-reactive protein (CRP) gene, (11) a polymorphism (e.g., GG genotype) at rs3087386 of DNA repair protein (Rev1) gene, (12) a polymorphism (e.g., GG genotype) at rs645419 of matrix metalloproteinase-12 (MMP12) gene, (13) a polymorphism (e.g., TT genotype) at rs763110 of Fas ligand (FasL) gene, (14) a polymorphism (e.g., GG genotype) at rs1052486 of HLA-B-associated transcript 3 (BAT3) gene, (15) a polymorphism (e.g., GG genotype) at rs1800797 of interleukin-6 (IL-6) gene, (16) a polymorphism (e.g., TT or TC genotype) at rs2070600 of advanced glycosylation end product-specific receptor (AGER) gene, (17) a polymorphism (e.g., CC or CT genotype) at rs2656069 of iron regulatory protein (IREB) gene, (18) a polymorphism (e.g., AA genotype) at rs16969968 of cholinergic receptor nicotinic alpha 3/5 (CHRNA3/5) gene, (19) a polymorphism (e.g., CC genotype) at rs402710 of telomerase reverse transcriptase (TERT) gene, and (20) a polymorphism (e.g., TT genotype) at rs7937 of cytochrome P450 family 2 subfamily A member 6 (CYP2A6) gene. In some embodiments, said first data set or said second data set comprises one or more single nucleotide polymorphisms (SNPs) selected from (i)-(vii) (e.g., from (iii)-(vii)): (i) at least one chronic obstructive pulmonary disease (COPD)-protective SNP: (ii) at least one COPD-susceptible SNP: (iii) at least one lung cancer-protective SNP: (iv) at least one lung cancer-susceptible SNP: (v) at least one lung cancer-susceptible, COPD-protective SNP: (vi) at least one lung cancer and COPD dual protective SNP; and (vii) at least one lung cancer and COPD dual susceptible SNP. In some embodiments, said at least one COPD-protective SNP of (i) comprises one or more SNPs selected from: a polymorphism in human Hedgehog interacting protein (HHIP) gene (e.g., at rs1489759 (e.g., GG)), a polymorphism in human Hedgehog interacting protein (HHIP) gene (e.g., at rs13141641 (e.g., CC)), a polymorphism in glycophorin A (GYPA) gene (e.g., at rs2202507 (e.g., GG)), a polymorphism in family with sequence similarity 13 member A (FAM13A) gene (e.g., at rs7671167 (e.g., CC)), a polymorphism in Ras and Rab interactor 3 (RIN3) gene (e.g., at rs754388 (e.g., CG or/and GG)), and a polymorphism in 5-hydroxytryptamine receptor 4 (HTR4) gene (e.g., at rs11168048 (e.g., CC)). In some embodiments, said at least one COPD-susceptible SNP of (ii) comprises one or more SNPs selected from: a polymorphism in deleted in liver cancer 1 (DLC1) gene (e.g., at rs58863591 (e.g., CT or/and TT)), and a polymorphism in a disintegrin and metalloproteinase 19 (ADAM19) gene (e.g., at rs1422795 (e.g., CC)). In some embodiments, said at least one lung cancer-protective SNP of (iii) comprises one or more SNPs selected from: a polymorphism in integrin alpha 11 (ITGA11) gene (e.g., at rs2306022 (e.g., TT or/and TC)), a polymorphism in C-reactive protein (CRP) gene (e.g., at rs2808630 (e.g., CC)), a polymorphism in DNA repair protein (Rev1) gene (e.g., at rs3087386 (e.g., GG)), a polymorphism in matrix metalloproteinase-12 (MMP12) gene (e.g., at rs645419 (e.g., GG)), and a polymorphism in Fas ligand (FasL) gene (e.g., at rs763110 (e.g., TT)). In some embodiments, said at least one lung cancer-susceptible SNP of (iv) comprises one or more SNPs selected from: a polymorphism in HLA-B-associated transcript 3 (BAT3) gene (e.g., at rs1052486 (e.g., GG)), and a polymorphism in interleukin-6 (IL-6) gene (e.g., at rs1800797 (e.g., GG)) In some embodiments, said at least one lung cancer-susceptible. COPD-protective SNP of (v) comprises a polymorphism in advanced glycosylation end product-specific receptor (AGER) gene (e.g., at rs2070600 (e.g., TT or/and TC)). In some embodiments, said at least one lung cancer and COPD dual protective SNP m of (vi) comprises a polymorphism in iron regulatory protein (IREB) gene (e.g., at rs2656069 (e.g., CC or/and CT)). In some embodiments, said at least one lung cancer and COPD dual susceptible SNP of (vii) comprises one or more SNPs selected from: a polymorphism in cholinergic receptor nicotinic alpha 3/5 (CHRNA3/5) gene (e.g., at rs16969968 (e.g., AA)), a polymorphism in telomerase reverse transcriptase (TERT) gene (e.g., at rs402710 (e.g., CC)), a polymorphism in cytochrome P450 family 2 subfamily A member 6 (CYP2A6) gene (e.g., at rs7937 (e.g., TT)). In some embodiments, said subject is determined to be predisposed to develop or die of lung cancer within a period of time (e.g., about 5 years). In some embodiments, said subject is determined to be predisposed to develop lung cancer with or without accompanying COPD within a period of time. In some embodiments, when said risk-value is 1-6, said subject is diagnosed with an aggressive lung cancer. In some embodiments, said subject has not been diagnosed with chronic obstructive pulmonary disease (COPD). In some embodiments, said subject has been diagnosed with (e.g., mild-moderate) chronic obstructive pulmonary disease (COPD). In some embodiments, regardless of whether said subject has been diagnosed with chronic obstructive pulmonary disease (COPD). In some embodiments, said subject is determined as having an intermediate risk of developing or dying of lung cancer. In some embodiments, said subject has or had an exposure to smoking (e.g., active or passive smoking) (e.g., tobacco smoking, such as cigarette smoking, pipe smoking, cigar smoking, use of chewing tobacco, or a combination thereof). In some embodiments, said subject is an active tobacco consumer or an active smoker (e.g., having a smoking status of at least 2 cigarettes per day, minimum equivalent to 5 pack years or on average 5 cigarettes per day for 20 years or 20 cigarettes per day for 5 years). In some embodiments, said bodily sample is selected from the group consisting of: a blood sample, a serum sample, a plasma sample, a saliva sample, a stool sample, a sputum sample, a urine sample, a semen sample, a transvaginal fluid sample, a cerebrospinal fluid sample, a sweat sample, a cell sample, and a tissue sample. In some embodiments, said pulmonary or respiratory disease or disorder is selected from the group consisting of lung cancer, chronic obstructive pulmonary disease (COPD), occupational chronic obstructive pulmonary disease (OCOPD), emphysema, and bodily sample(s). In some embodiments, said first data set or said second data set indicates a genetic predisposition (e.g., predominantly protective, predominantly susceptible, or neutral) of said subject to said pulmonary or respiratory disease or disorder. In some embodiments, said analyzing comprises sequencing ribonucleic acid (RNA) molecules obtained or derived from said bodily sample to yield said data set, wherein said data set comprises sequencing reads. In some embodiments, said analyzing comprises performing reverse transcription polymerase chain reaction (RT-PCR) on ribonucleic acid (RNA) molecules obtained or derived from said bodily sample to yield said data set, wherein said data set comprises sequencing reads. In some embodiments, said analyzing comprises reverse transcribing ribonucleic acid (RNA) molecules obtained or derived from said bodily sample to yield complementary deoxyribonucleic acid (cDNA) molecules, and sequencing at least a portion of said cDNA molecules to yield said data set, wherein said data set comprises sequencing reads. In some embodiments, said analyzing comprises reverse transcribing ribonucleic acid (RNA) molecules obtained or derived from said bodily sample to yield complementary deoxyribonucleic acid (cDNA) molecules, and assaying at least a portion of said cDNA molecules by quantitative polymerase reaction (qPCR) to yield said data set, wherein said data set comprises sequencing reads. In some embodiments, said at least one biomarker comprises analysis of proteins, antibodies, or nucleic acids (e.g., liquid or tumor DNA, tumor RNA) obtained from said subject. In some embodiments, said risk associated with said pulmonary nodule is quantified in terms of the growth rate or malignancy of said pulmonary nodule. In some embodiments, determining said risk associated with said pulmonary nodule leads to improved clinical outcomes. In some embodiments, said improved clinical outcome is improvement in AUC. In some embodiments, said improved clinical outcome is more accurate reclassification of intermediate nodules as benign or malignant. In some embodiments, said improved clinical outcome is lower surgery rate for benign nodules. In some embodiments, the method further comprises analyzing a clinical health parameter selected from the group consisting of: age, gender, smoking status, pack years, self-reported comorbidity, spirometric-defined comorbidity, history of chronic obstructive pulmonary disease (COPD). CT evidence of emphysema, forced expiratory volume (FEV1). Forced vital capacity (FVC), family history of lung cancer, body mass index (BMI), lung function, lung cancer histology, lung cancer stage, and surgery.

Another aspect of the present disclosure provides a method of treating a pulmonary or respiratory disease or disorder of a subject, comprising: (a) diagnosing said pulmonary or respiratory disease or disorder of said subject, according to methods as disclosed herein and (b) treating said subject for said pulmonary or respiratory disease or disorder.

Another aspect of the present disclosure provides a method of treating a pulmonary or respiratory disease or disorder of a subject, comprising: treating said subject for said pulmonary or respiratory disease or disorder, which subject has been diagnosed with or exhibits at least one symptom associated with said pulmonary or respiratory disease or disorder of said subject, according to methods as disclosed herein.

Another aspect of the present disclosure provides a method for processing or analyzing a bodily sample of a subject, comprising: (a) analyzing said bodily sample to yield a data set comprising at least one genetic polymorphism in said bodily sample, wherein said at least one genetic polymorphism is associated with a pulmonary or respiratory disease or disorder; (b) computer processing said data set from (a) and a clinical health parameter of said subject to determine a risk-value of said pulmonary or respiratory disease or disorder in said subject; and (c) electronically outputting a report that identifies said risk-value of said pulmonary or respiratory disease or disorder in said subject determined in (b). In some embodiments, in (b), said risk-value of said pulmonary or respiratory disease or disorder in said subject is determined at an Area Under Curve (AUC) at least 1.01-, 1.02-, 1.03-, 1.04-, 1.05-, or 1.06-fold greater than a corresponding AUC achieved with said clinical score In some embodiments, in (b), said risk-value of said pulmonary or respiratory disease or disorder in said subject is determined at a sensitivity at least 1.01-, 1.02-, 1.03-, 1.04-, 1.05-, or 1.06-fold greater than a corresponding sensitivity achieved with said clinical score. In some embodiments, in (b), said risk-value of said pulmonary or respiratory disease or disorder in said subject is determined at a specificity at least 1.01-, 1.02-, 1.03-, 1.04-, 1.05-, or 1.06-fold greater than a corresponding specificity achieved with said clinical score. In some embodiments, in (b), said first risk-value of said pulmonary or respiratory disease or disorder in said subject is determined at: a sensitivity at least 1.01-, 1.02-, 1.03-, 1.04-, 1.05-, or 1.06-fold greater than a corresponding sensitivity achieved with said clinical score, and a specificity no less than a corresponding specificity achieved with said clinical score. In some embodiments, in (b), said first risk-value of said pulmonary or respiratory disease or disorder in said subject is determined at: a sensitivity no less than a corresponding sensitivity achieved with said clinical score, and a specificity at least 1.01-, 1.02-, 1.03-, 1.04-, 1.05-, or 1.06-fold greater than a corresponding specificity achieved with said clinical score. In some embodiments, in (b), said first risk-value of said pulmonary or respiratory disease or disorder in said subject is determined at: a sensitivity at least 1.01-, 1.02-, 1.03-, 1.04-, 1.05-, or 1.06-fold greater than a corresponding sensitivity achieved with said clinical score, and a specificity at least 1.01-, 1.02-, 1.03-, 1.04-, 1.05-, or 1.06-fold greater than a corresponding specificity achieved with said clinical score. In some embodiments, said risk-value indicates a presence, a reduced risk, or an elevated risk of said pulmonary or respiratory disease or disorder. In some embodiments, said clinical health parameter of said subject comprises at least one member selected from the group consisting of age, gender, smoking status, pack years, self-reported comorbidity, spirometric-defined comorbidity, history of chronic obstructive pulmonary disease (COPD), family history of lung cancer, body mass index (BMI), lung function, lung cancer histology, lung cancer stage, and surgery. In some embodiments, the method further comprises determining said clinical score. In some embodiments, said at least one genetic polymorphism comprises at least two, at least five, at least ten, or at least fifteen single nucleotide polymorphisms (SNPs) selected from: a polymorphism at rs1489759 of Hedgehog Interacting Protein (HHIP) gene, a polymorphism at rs13141641 of Hedgehog Interacting Protein (HHIP) gene, a polymorphism at rs2202507 of Glycophorin A (GYPA) gene, a polymorphism at rs7671167 of Family with Sequence Similarity 13 Member A (FAM13A) gene, a polymorphism at rs754388 of Ras and Rab Interactor 3 (RIN3) gene, a polymorphism at rs11168048 of 5-Hydroxytryptamine Receptor 4 (HTR4) gene, a polymorphism at rs58863591 of Deleted in Liver Cancer 1 (DL (1) gene, a polymorphism at rs1422795 of A Disintegrin and Metalloproteinase 19 (ADAM19) gene, a polymorphism at rs2306022 of Integrin Alpha 11 (ITGA11) gene, a polymorphism at rs2808630 of C-Reactive Protein (CRP) gene, a polymorphism at rs3087386 of DNA Repair Protein (Rev1) gene, a polymorphism at rs645419 of Matrix Metalloproteinase-12 (MMP12) gene, a polymorphism at rs763110 of Fas Ligand (FasL) gene, a polymorphism at rs1052486 of HLA-B-Associated Transcript 3 (BAT3) gene, a polymorphism at rs1800797 of Interleukin-6 (IL-6) gene, a polymorphism at rs2070600 of Advanced Glycosylation End Product-Specific Receptor (AGER) gene, a polymorphism at rs2656069 of Iron Regulatory Protein (IREB) gene, a polymorphism at rs16969968 of Cholinergic Receptor Nicotinic Alpha 3/5 (CHRNA3/5) gene, a polymorphism at rs402710 of Telomerase Reverse Transcriptase (TERT) gene, and a polymorphism at rs7937 of Cytochrome P450 Family 2 Subfamily A Member 6 (CYP2A6) gene.

Another aspect of the present disclosure provides a method for processing or analyzing a bodily sample of a subject, comprising: (a) analyzing said bodily sample to yield a data set comprising at least one genetic polymorphism in said bodily sample, wherein said at least one genetic polymorphism is associated with a pulmonary or respiratory disease or disorder; (b) computer processing said data set from (a) to determine a risk-value of said pulmonary or respiratory disease or disorder in said subject at an accuracy of at least about 80%, wherein said accuracy is a percentage of a plurality of independent test samples corresponding to (1) a first plurality of subjects having said pulmonary or respiratory disease or disorder relative to (2) a second plurality of subjects who do not have said pulmonary or respiratory disease or disorder or who have negative clinical indications for said pulmonary or respiratory disease or disorder, that is correctly determined to have, not have, be at risk of, or not be at risk of, said pulmonary or respiratory disease or disorder; and (c) electronically outputting a report that identifies said risk-value of said pulmonary or respiratory disease or disorder in said subject determined in (b). In some embodiments, in (b), said risk-value of said pulmonary or respiratory disease or disorder in said subject is determined at an accuracy of at least about 60%. In some embodiments, in (b), said risk-value of said pulmonary or respiratory disease or disorder in said subject is determined at an accuracy of at least about 70%. In some embodiments, said accuracy comprises a ratio of (1) a first sum of true positive cases and true negative cases, to (2) a second sum of true positive cases, true negative cases, false positive cases, and false negative cases. In some embodiments, in (b), said risk-value is determined at a sensitivity of at least about 80%. In some embodiments, in (b), said risk-value of said pulmonary or respiratory disease or disorder in said subject is determined at a sensitivity of at least about 90%. In some embodiments, in (b), said risk-value of said pulmonary or respiratory disease or disorder in said subject is determined at a sensitivity of at least about 95%. In some embodiments, said sensitivity comprises a ratio of (1) a number of true positive cases, to (2) a sum of true positive cases and false negative cases. In some embodiments, said sensitivity is a percentage of a plurality of independent test samples corresponding to (1) a first plurality of subjects receiving an elevated risk-value of said pulmonary or respiratory disease or disorder, relative to (2) a second plurality of subjects having said pulmonary or respiratory disease or disorder that is correctly determined to have or be at risk of said pulmonary or respiratory disease or disorder. In some embodiments, in (b), said risk-value is determined at a specificity of at least about 80%. In some embodiments, in (b), said risk-value of said pulmonary or respiratory disease or disorder in said subject is determined at a specificity of at least about 90% In some embodiments, in (b), said risk-value of said pulmonary or respiratory disease or disorder in said subject is determined at a specificity of at least about 95%. In some embodiments, said specificity comprises a ratio of (1) a number of true negative cases, to (2) a sum of true negative cases and false positive cases. In some embodiments, said specificity is a percentage of a plurality of independent test samples corresponding to (1) a first plurality of subjects receiving an non-elevated risk-value of said pulmonary or respiratory disease or disorder, relative to (2) a second plurality who do not have said pulmonary or respiratory disease or disorder or who have negative clinical indications for said pulmonary or respiratory disease or disorder, that is correctly determined to not have or not be at risk of said pulmonary or respiratory disease or disorder. In some embodiments, in (b), said risk-value is determined at an Area Under Curve (AUC) of at least about 80%. In some embodiments, in (b), said risk-value of said pulmonary or respiratory disease or disorder in said subject is determined at an AUC of at least about 90%. In some embodiments, in (b), said risk-value of said pulmonary or respiratory disease or disorder in said subject is determined at an AUC of at least about 95%. In some embodiments, said at least one genetic polymorphism comprises at least two, at least five, at least ten, or at least fifteen single nucleotide polymorphisms (SNPs) selected from: a polymorphism at rs1489759 of Hedgehog Interacting Protein (HHIP) gene, a polymorphism at rs13141641 of Hedgehog Interacting Protein (HHIP) gene, a polymorphism at rs2202507 of Glycophorin A (GYPA) gene, a polymorphism at rs7671167 of Family with Sequence Similarity 13 Member A (FAM13A) gene, a polymorphism at rs754388 of Ras and Rab Interactor 3 (RIN3) gene, a polymorphism at rs11168048 of 5-Hydroxytryptamine Receptor 4 (HTR4) gene, a polymorphism at rs58863591 of Deleted in Liver Cancer 1 (DL (1) gene, a polymorphism at rs1422795 of A Disintegrin and Metalloproteinase 19 (ADAM19) gene, a polymorphism at rs2306022 of Integrin Alpha 11 (ITGA11) gene, a polymorphism at rs2808630 of C-Reactive Protein (CRP) gene, a polymorphism at rs3087386 of DNA Repair Protein (Rev1) gene, a polymorphism at rs645419 of Matrix Metalloproteinase-12 (MMP12) gene, a polymorphism at rs763110 of Fas Ligand (FasL) gene, a polymorphism at rs1052486 of HLA-B-Associated Transcript 3 (BAT3) gene, a polymorphism at rs1800797 of Interleukin-6 (IL-6) gene, a polymorphism at rs2070600 of Advanced Glycosylation End Product-Specific Receptor (AGER) gene, a polymorphism at rs2656069 of Iron Regulatory Protein (IREB) gene, a polymorphism at rs16969968 of Cholinergic Receptor Nicotinic Alpha 3/5 (CHRNA3/5) gene, a polymorphism at rs402710 of Telomerase Reverse Transcriptase (TERT) gene, and a polymorphism at rs7937 of Cytochrome P450 Family 2 Subfamily A Member 6 (CYP2A6) gene. In some embodiments, said risk-value indicates a presence, a reduced risk, or an elevated risk of said pulmonary or respiratory disease or disorder.

Another aspect of the present disclosure provides a method for processing or analyzing a bodily sample of a subject, comprising: analyzing said bodily sample to yield a data set comprising at least one genetic polymorphism in said bodily sample, wherein said at least one genetic polymorphism is associated with a pulmonary or respiratory disease or disorder and comprises at least two, at least five, at least ten, or at least fifteen single nucleotide polymorphisms (SNPs) selected from: a polymorphism at rs1489759 of Hedgehog Interacting Protein (HHIP) gene, a polymorphism at rs13141641 of Hedgehog Interacting Protein (HHIP) gene, a polymorphism at rs2202507 of Glycophorin A (GYPA) gene, a polymorphism at rs7671167 of Family with Sequence Similarity 13 Member A (FAM13A) gene, a polymorphism at rs754388 of Ras and Rab Interactor 3 (RIN3) gene, a polymorphism at rs11168048 of 5-Hydroxytryptamine Receptor 4 (HTR4) gene, a polymorphism at rs58863591 of Deleted in Liver Cancer 1 (DI, (1) gene, a polymorphism at rs1422795 of A Disintegrin and Metalloproteinase 19 (ADAM19) gene, a polymorphism at rs2306022 of Integrin Alpha 11 (ITGA11) gene, a polymorphism at rs2808630 of C-Reactive Protein (CRP) gene, a polymorphism at rs3087386 of DNA Repair Protein (Rev1) gene, a polymorphism at rs645419 of Matrix Metalloproteinase-12 (MMP12) gene, a polymorphism at rs763110 of Fas Ligand (FasL) gene, a polymorphism at rs1052486 of HLA-B-Associated Transcript 3 (BAT3) gene, a polymorphism at rs1800797 of Interleukin-6 (IL-6) gene, a polymorphism at rs2070600 of Advanced Glycosylation End Product-Specific Receptor (AGER) gene, a polymorphism at rs2656069 of Iron Regulatory Protein (IREB) gene, a polymorphism at rs16969968 of Cholinergic Receptor Nicotinic Alpha 3/5 (CHRNA3/5) gene, a polymorphism at rs402710 of Telomerase Reverse Transcriptase (TERT) gene, and a polymorphism at rs7937 of Cytochrome P450 Family 2 Subfamily A Member 6 (CYP2A6) gene. In some embodiments, said at least one genetic polymorphism comprises 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, or all 20 single nucleotide polymorphisms (SNPs) selected from: a polymorphism (e.g., GG genotype) at rs1489759 of human Hedgehog interacting protein (HHIP) gene, a polymorphism (e.g., CC genotype) at rs13141641 of human Hedgehog interacting protein (HHIP) gene, a polymorphism (e.g., GG or/and CC genotype) at rs2202507 of glycophorin A (GYPA) gene, a polymorphism (e.g., CC genotype) at rs7671167 of family with sequence similarity 13 member A (FAM13A) gene, a polymorphism (e.g., CG or GG genotype) at rs754388 of Ras and Rab interactor 3 (RIN3) gene, a polymorphism (e.g., CC genotype) at rs11168048 of 5-hydroxytryptamine receptor 4 (HTR4) gene, a polymorphism (e.g., CT, or TT genotype) at rs58863591 of deleted in liver cancer 1 (DI, (1) gene, a polymorphism (e.g., CC genotype) at rs1422795 in a disintegrin and metalloproteinase 19 (ADAM19) gene, a polymorphism (e.g., TT or TC genotype) at rs2306022 integrin alpha 11 (ITGA11) gene, a polymorphism (e.g., CC genotype) at rs2808630 of C-reactive protein (CRP) gene, a polymorphism (e.g., GG genotype) at rs3087386 of DNA repair protein (Rev1) gene, a polymorphism (e.g., GG genotype) at rs645419 of matrix metalloproteinase-12 (MMP12) gene, a polymorphism (e.g., TT genotype) at rs763110 of Fas ligand (FasL) gene, a polymorphism (e.g., GG genotype) at rs1052486 of HLA-B-associated transcript 3 (BAT3) gene, a polymorphism (e.g., GG genotype) at rs1800797 of interleukin-6 (IL-6) gene, a polymorphism (e.g., TT or TC genotype) at rs2070600 of advanced glycosylation end product-specific receptor (AGER) gene, a polymorphism (e.g., CC or CT genotype) at rs2656069 of iron regulatory protein (IREB) gene, a polymorphism (e.g., AA genotype) at rs16969968 of cholinergic receptor nicotinic alpha 3/5 (CHRNA3/5) gene, a polymorphism (e.g., CC genotype) at rs402710 of telomerase reverse transcriptase (TERT) gene, and a polymorphism (e.g., TT genotype) at rs7937 of cytochrome P450 family 2 subfamily A member 6 (CYP2A6) gene. In some embodiments, wherein said at least one genetic polymorphism comprises one or more single nucleotide polymorphisms (SNPs) selected from (i)-(vii) (e.g., from (iii)-(vii)): at least one chronic obstructive pulmonary disease (COPD)-protective SNP: (ii) at least one COPD-susceptible SNP: (iii) at least one lung cancer-protective SNP: (iv) at least one lung cancer-susceptible SNP: (v) at least one lung cancer-susceptible, COPD-protective SNP: (vi) at least one lung cancer and COPD dual protective SNP; and (vii) at least one lung cancer and COPD dual susceptible SNP. In some embodiments, said at least one COPD-protective SNP of (i) comprises one or more SNPs selected from: a polymorphism in human Hedgehog interacting protein (HHIP) gene (e.g., at rs1489759 (e.g., GG)), a polymorphism in human Hedgehog interacting protein (HHIP) gene (e.g., at rs13141641 (e.g., CC)), a polymorphism in glycophorin A (GYPA) gene (e.g., at rs2202507 (e.g., GG)), a polymorphism in family with sequence similarity 13 member A (FAM13A) gene (e.g., at rs7671167 (e.g., CC)), a polymorphism in Ras and Rab interactor 3 (RIN3) gene (e.g., at rs754388 (e.g., CG or/and GG)), and a polymorphism in 5-hydroxytryptamine receptor 4 (HTR4) gene (e.g., at rs11168048 (e.g., CC)). In some embodiments, said at least one COPD-susceptible SNP of (ii) comprises one or more SNPs selected from: a polymorphism in deleted in liver cancer 1 (DLC1) gene (e.g., at rs58863591 (e.g., CT or/and TT)), and a polymorphism in a disintegrin and metalloproteinase 19 (ADAM19) gene (e.g., at rs1422795 (e.g., CC)). In some embodiments, said at least one lung cancer-protective SNP of (iii) comprises one or more SNPs selected from: a polymorphism in integrin alpha 11 (ITGA11) gene (e.g., at rs2306022 (e.g., TT or/and TC)), a polymorphism in C-reactive protein (CRP) gene (e.g., at rs2808630 (e.g., CC)), a polymorphism in DNA repair protein (Rev1) gene (e.g., at rs3087386 (e.g., GG)), a polymorphism in matrix metalloproteinase-12 (MMP12) gene (e.g., at rs645419 (e.g., GG)), and a polymorphism in Fas ligand (FasL) gene (e.g., at rs763110 (e.g., TT)). In some embodiments, said at least one lung cancer-susceptible SNP of (iv) comprises one or more SNPs selected from: a polymorphism in HLA-B-associated transcript 3 (BAT3) gene (e.g., at rs1052486 (e.g., GG)), and a polymorphism in interleukin-6 (IL-6) gene (e.g., at rs1800797 (e.g., GG)) In some embodiments, said at least one lung cancer-susceptible, COPD-protective SNP of (v) comprises a polymorphism in advanced glycosylation end product-specific receptor (AGER) gene (e.g., at rs2070600 (e.g., TT or/and TC)). In some embodiments, said at least one lung cancer and COPD dual protective SNP m of (vi) comprises a polymorphism in iron regulatory protein (IREB) gene (e.g., at rs2656069 (e.g., CC or/and CT)). In some embodiments, said at least one lung cancer and COPD dual susceptible SNP of (vii) comprises one or more SNPs selected from: a polymorphism in cholinergic receptor nicotinic alpha 3/5 (CHRNA3/5) gene (e.g., at rs16969968 (e.g., AA)), a polymorphism in telomerase reverse transcriptase (TERT) gene (e.g., at rs402710 (e.g., CC)), a polymorphism in cytochrome P450 family 2 subfamily A member 6 (CYP2A6) gene (e.g., at rs7937 (e.g., TT)). In some embodiments and 128-136, wherein, in (b), said subject is determined to be predisposed to develop or die of lung cancer within a period of time (e.g., about 5 years). In some embodiments and 128-137, wherein, in (b), said subject is determined to be predisposed to develop lung cancer with or without accompanying COPD within a period of time. In some embodiments, wherein when said risk-value is 1-6, said subject is diagnosed with an aggressive lung cancer. In some embodiments, said subject has not been diagnosed with chronic obstructive pulmonary disease (COPD). In some embodiments, said subject has been diagnosed with (e.g., mild-moderate) chronic obstructive pulmonary disease (COPD). In some embodiments, regardless whether said subject has been diagnosed with chronic obstructive pulmonary disease (COPD). In some embodiments, said subject is determined as having an intermediate risk of developing or dying of lung cancer (e.g., about 0.5% to about 2.0% in quintiles Q2 to Q4). In some embodiments, said subject has or had an exposure to smoking (e.g., active or passive smoking) (e.g., tobacco smoking, such as cigarette smoking, pipe smoking, cigar smoking, use of chewing tobacco, or a combination thereof). In some embodiments, said subject is an active tobacco consumer or an active smoker (e.g., having a smoking status of at least 2 cigarettes per day, minimum equivalent to 5 pack years or on average 5 cigarettes per day for 20 years or 20 cigarettes per day for 5 years). In some embodiments, said bodily sample is selected from the group consisting of: a blood sample, a serum sample, a plasma sample, a saliva sample, a buccal sample, a stool sample, a sputum sample, a urine sample, a semen sample, a transvaginal fluid sample, a cerebrospinal fluid sample, a sweat sample, a cell sample, and a tissue sample. In some embodiments, said bodily sample comprises (DNA, RNA, cell-free DNA, cell-free RNA, proteins, lipids, metabolites, cells, tissues, etc.) In some embodiments, said pulmonary or respiratory disease or disorder is selected from the group consisting of lung cancer, chronic obstructive pulmonary disease (COPD), occupational chronic obstructive pulmonary disease (OCOPD), and emphysema. In some embodiments, said at least one genetic polymorphism indicates a genetic predisposition (e.g., predominantly protective, predominantly susceptible, or neutral) of said subject to said pulmonary or respiratory disease or disorder. In some embodiments, said analyzing comprises sequencing ribonucleic acid (RNA) molecules obtained or derived from said bodily sample to yield said data set, wherein said data set comprises sequencing reads. In some embodiments 83, wherein said analyzing comprises performing reverse transcription polymerase chain reaction (RT-PCR) on ribonucleic acid (RNA) molecules obtained or derived from said bodily sample to yield said data set, wherein said data set comprises sequencing reads. In some embodiments, said analyzing comprises reverse transcribing ribonucleic acid (RNA) molecules obtained or derived from said bodily sample to yield complementary deoxyribonucleic acid (cDNA) molecules, and sequencing at least a portion of said cDNA molecules to yield said data set, wherein said data set comprises sequencing reads. In some embodiments, said analyzing comprises reverse transcribing ribonucleic acid (RNA) molecules obtained or derived from said bodily sample to yield complementary deoxyribonucleic acid (cDNA) molecules, and assaying at least a portion of said cDNA molecules by quantitative polymerase reaction (qPCR) to yield said data set, wherein said data set comprises sequencing reads.

Another aspect of the present disclosure provides a method of treating a pulmonary or respiratory disease or disorder of a subject, comprising: diagnosing said pulmonary or respiratory disease or disorder of said subject, according to In some embodiments; and treating said subject for said pulmonary or respiratory disease or disorder.

Another aspect of the present disclosure provides a method of treating a pulmonary or respiratory disease or disorder of a subject, comprising: treating said subject for said pulmonary or respiratory disease or disorder, which subject has been diagnosed with or exhibits at least one symptom associated with said pulmonary or respiratory disease or disorder of said subject, according to methods as disclosed herein.

Another aspect of the present disclosure provides a kit for processing or analyzing a bodily sample of a subject, comprising: a set of probes for identifying a presence, absence, or relative amount of a set of genomic regions associated with lung cancer, chronic obstructive pulmonary disease (COPD), occupational chronic obstructive pulmonary disease (OCOPD), and emphysema in said bodily sample or a derivative thereof, wherein said set of probes identify at least two, at least five, at least ten, or at least fifteen single nucleotide polymorphisms (SNPs) selected from: a polymorphism at rs1489759 of Hedgehog Interacting Protein-1 (HHIP) gene, a polymorphism at rs13141641 of Hedgehog Interacting Protein-2 (HHIP) gene, a polymorphism at rs2202507 of Glycophorin A (GYPA) gene, a polymorphism at rs7671167 of Family with Sequence Similarity 13 Member A (FAM13A) gene, a polymorphism at rs754388 of Ras and Rab Interactor 3 (RIN3) gene, a polymorphism at rs11168048 of 5-Hydroxytryptamine Receptor 4 (HTR4) gene, a polymorphism at rs58863591 of Deleted in Liver Cancer 1 (DI, (1) gene, a polymorphism at rs1422795 of A Disintegrin and Metalloproteinase 19 (ADAM19) gene, a polymorphism at rs2306022 of Integrin Alpha 11 (ITGA11) gene, a polymorphism at rs2808630 of C-Reactive Protein (CRP) gene, a polymorphism at rs3087386 of DNA Repair Protein (Rev1) gene, a polymorphism at rs645419 of Matrix Metalloproteinase-12 (MMP12) gene, a polymorphism at rs763110 of Fas Ligand (FasL) gene, a polymorphism at rs1052486 of HLA-B-Associated Transcript 3 (BAT3) gene, a polymorphism at rs1800797 of Interleukin-6 (IL-6) gene, a polymorphism at rs2070600 of Advanced Glycosylation End Product-Specific Receptor (AGER) gene, a polymorphism at rs2656069 of Iron Regulatory Protein (IREB) gene, a polymorphism at rs16969968 of Cholinergic Receptor Nicotinic Alpha 3/5 (CHRNA3/5) gene, a polymorphism at rs402710 of Telomerase Reverse Transcriptase (TERT) gene, and a polymorphism at rs7937 of Cytochrome P450 Family 2 Subfamily A Member 6 (CYP2A6) gene. In some embodiments, the kit further comprises a label or package insert that provides information on how said set of probes are used.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 illustrates a non-limiting example of a computing device: in this case, a device with one or more processors, memory, storage, and a network interface.

FIG. 2 illustrates a non-limiting example of a web/mobile application provision system: in this case, a system providing browser-based and/or native mobile user interfaces.

FIG. 3 illustrates a non-limiting example of a cloud-based web/mobile application provision system; in this case, a system comprising an elastically load balanced, auto-scaling web server and application server resources as well synchronously replicated databases.

FIG. 4 illustrates the genetic study subgroup from the NLST. Legends: NLST: National Lung Screening Trial; ACRIN: American College of Radiology, Imaging Network; NCI: National Cancer Institute; NZ: New Zealand.

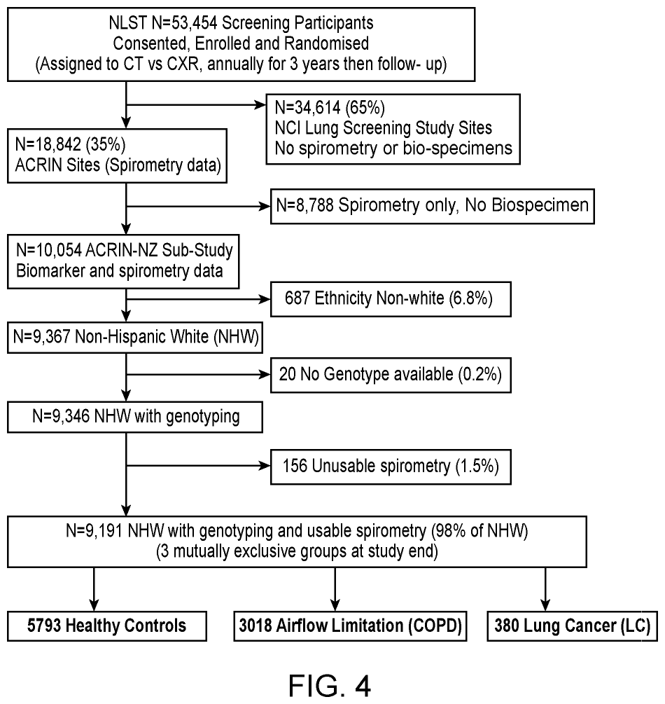

FIG. 5 illustrates phenotypic groups according to baseline lung function and subsequent development of lung cancer (mutually exclusive groups) in 9,191 Non-Hispanic Whites. Legends: At baseline 5977 had no airflow limitation (No COPD) and 3214 had airflow limitation (35%) according to pre-bronchodilator spirometry (Table 4 and Table 5).

FIG. 6 illustrates a schematic representation of the risk genotype effects for COPD and Lung cancer.

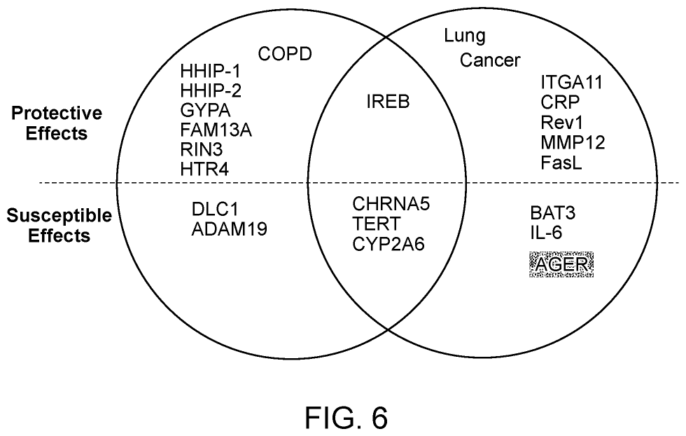

FIG. 7 illustrates the genetic study subgroup from the NLST. Legends: NLST: National Lung Screening Trial; ACRIN: American College of Radiology, Imaging Network; NCI: National Cancer Institute; NZ: New Zealand; NHW: Non-Hispanic Whites; SNP: Single Nucleotide Polymorphism; CT: computerized tomography; and CXR: Chest X-ray.

FIG. 8 illustrates the 5-year risk of developing (dotted) or dying (solid) from lung cancer according to the gene-based risk tertiles.

FIG. 9 illustrates lung cancer prevalence per 1000 using the gene-based (solid) and PLCO_{M2012} (dotted) models according to quintiles.

FIGS. 10A-B illustrates proportion of subjects who change their risk group after reclassification from PLCOM2012 lung cancer risk recalculated according to the composite gene-based risk. FIG. 10A: reclassification of tertiles of risk. FIG. 10B: reclassification of quintiles of risk.

FIG. 11 illustrates outcomes for lung cancer according to the lung cancer polygenic risk score (PRS) groups (Table 11).

FIG. 12 illustrates cause-specific mortality analyzed across PRS groups as: FIG. 12A: percentage of all deaths; and FIG. 12B: deaths per 1000 within each PRS risk group (crude tertile, Table 11).

FIG. 13 illustrates risk difference for lung cancer deaths averted according to PRS risk grouping after screening randomization (Table 11).

FIG. 14 illustrates odds of lung cancer deaths referenced against the low PRS risk group and adjusted according to clinical factors, clinical score and GOLD 1-4 status.

FIG. 15 illustrates a consort figure of the genetic study subgroup from the study of Example 4.

FIG. 16 illustrates outcomes for lung cancer according to the lung cancer polygenic risk score (PRS) groups.

FIG. 17 illustrates risk difference for (a) lung cancer lethality and (b) lung cancer deaths averted per 1000 screened according to polygenic risk score (PRS) grouping after screening randomization.

FIG. 18 illustrates cause-specific mortality across polygenic risk score (PRS) groups as (a) percentage of all deaths and (b) deaths per 1000 within each PRS group.

FIG. 19 illustrates odds of lung cancer deaths referenced against the low polygenic risk score (PRS) group and adjusted according to clinical factors, clinical score and GOLD 1-4 status.

FIG. 20 illustrates lung cancer prevalence per 1000 using the gene-based (solid line) and PLCO_{M2012} (dashed line) models according to quintiles of 6-year risk of developing lung cancer.

The novel features of the disclosure are set forth with particularity in the appended claims. A better understanding of the features and advantages of the present disclosure will be obtained by reference to the following detailed description that sets forth illustrative embodiments.

### DETAILED DESCRIPTION

### Overview

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Described herein, in some embodiments, are methods for processing or analyzing a bodily sample in a subject. In some embodiments, the method entails determining a risk associated with a pulmonary or respiratory disease or disorder in a subject. In some embodiments, the method identifies or quantifies at least one genetic polymorphism in a biological sample obtained from the subject. In some embodiments, the method entails analyzing the bodily sample to yield a first data set comprising at least one genetic polymorphism in a bodily sample. The genetic polymorphism maybe associated with a first pulmonary or respiratory disease or disorder. In some embodiments, the method comprises analyzing the bodily sample to yield a second data set comprising at least one genetic polymorphism in the bodily sample. The bodily sample may be selected from the group consisting of: a blood sample, a scrum sample, a plasma sample, a saliva sample, a stool sample, a sputum sample, a urine sample, a semen sample, a transvaginal fluid sample, a cerebrospinal fluid sample, a sweat sample, a cell sample, and a tissue sample. In some embodiments, the at least one genetic polymorphism is associated with a second pulmonary or respiratory disease or disorder. In some embodiments, a polymorphism profile is established based on the analysis of the first data set. In some embodiments, a polymorphism profile is established based on the analysis of the second data set. In some embodiments, the method comprises imaging a pulmonary nodule from the subject to determine at least one characteristic associated with said image of the pulmonary nodule. In some embodiments, the method comprises obtaining data with respect to at least one biomarker of the subject. In some embodiments, the method further comprises computer processing the first data set and the second data set to yield a third data set. In some embodiments, the method further comprises analyzing the third data set in conjunction with the at least one characteristic of the pulmonary nodule with the at least one biomarker to determine the risk associated with the pulmonary nodule. In some embodiments, the method comprises electronically outputting a report that identifies the risk presented by the pulmonary nodule. In some embodiments, the polymorphism profile comprises a sensitivity or a specificity determined according to a receiver operator characteristic (ROC) curve having an area under curve (AUC) value. In some embodiments, the method determines an early diagnosis or a therapeutic intervention for treating a subject who is at risk of developing lung cancer or a subject who has already developed lung cancer. In some embodiments, the pulmonary nodule is detected during a procedure or visit related to lung cancer screening. In some embodiments, the pulmonary nodule is detected during a procedure or visit that is not related to lung cancer screening. In some embodiments, the pulmonary nodule is an intermediate risk nodule. In some embodiments, the pulmonary nodule is an indeterminate bodily sample. In some embodiments, the first pulmonary or respiratory disease or disorder is lung cancer. In some embodiments, the second pulmonary or respiratory disease or disorder is lung cancer. In some embodiments, the first pulmonary or respiratory disease or disorder is airflow limitation or chronic obstructive pulmonary disease (COPD). In some embodiments, the second pulmonary or respiratory disease or disorder is airflow limitation or chronic obstructive pulmonary disease (COPD).

Also described herein, in some embodiments, are methods for improving performance of determining a lung cancer-associated risk of a subject by incorporating analysis of the at least one genetic polymorphism of the subject. In some embodiments, the method comprises identifying or quantifying the at least one genetic polymorphism in a biological sample obtained from said subject in need thereof to obtain a polymorphism profile of said subject. In some embodiments, the method identifies or quantifies at least one genetic polymorphism in a biological sample obtained from the subject. In some embodiments, the method entails analyzing the bodily sample to yield a first data set comprising at least one genetic polymorphism in a bodily sample. The genetic polymorphism maybe associated with a first pulmonary or respiratory disease or disorder. In some embodiments, the method comprises analyzing the bodily sample to yield a second data set comprising at least one genetic polymorphism in the bodily sample. In some embodiments, the at least one genetic polymorphism is associated with a second pulmonary or respiratory disease or disorder. In some embodiments, a polymorphism profile is established based on the analysis of the first data set. In some embodiments, a polymorphism profile is established based on the analysis of the second data set. In some embodiments, the method determines, taking into consideration the polymorphism profile, a level of the lung cancer-associated risk of the subject such that said determination is characterized by a performance rate higher than a corresponding performance rate of a corresponding determination without identifying or quantifying said at least one genetic polymorphism. In some embodiments, the method determines: a first performance rate higher than a corresponding first performance rate of a corresponding determination without identifying or quantifying said at least one genetic polymorphism; and a second performance rate no less (or higher) than a corresponding second performance rate of said corresponding determination. In some embodiments, the method generates an AUC higher than a corresponding AUC of the corresponding determination without identifying or quantifying said at least one genetic polymorphism. In some embodiments, the method generates an increased specificity compared to a corresponding specificity of the corresponding determination without identifying or quantifying said at least one genetic polymorphism. In some embodiments, the method generates an increased sensitivity compared to a corresponding sensitivity of the corresponding determination without identifying or quantifying said at least one genetic polymorphism. In some embodiments, the method further identifies or quantifies at least one clinical variable (e.g., comprising one or more items selected from: age, gender, smoking status, pack years, self-reported comorbidity, spirometric-defined comorbidity, history of chronic obstructive pulmonary disease (COPD), family history of lung cancer, body mass index (BMI), lung function, lung cancer histology, lung cancer stage, or surgery). In some embodiments, the method determines the lung cancer-associated risk by analyzing both the polymorphism profile and the at least one clinical variable. In some embodiments, the method further determines an early diagnosis or therapeutic intervention (e.g., effective against lung cancer) for said subject.

In some embodiments, the method identifies two categories of polymorphisms: those associated with a reduced risk of developing lung cancer, which can be termed protective polymorphisms, and those associated with an increased risk of developing lung cancer known as susceptibility polymorphism. In some embodiments, the method comprises assessing a subject's risk of developing a pulmonary or respiratory disease or disorder by determining the presence or absence of at least one protective polymorphism associated with a reduced risk of developing lung cancer and/or the presence or absence of at least one susceptibility polymorphism associated with an increased risk of developing lung cancer. The presence of one or more of said protective polymorphisms may be indicative of a reduced risk of developing lung cancer. The absence of at least one protective polymorphism in combination with the presence of at least one susceptibility polymorphism may be indicative of an increased risk of developing lung cancer.

The present disclosure provides methods for determining the risk associated with a pulmonary nodule. In some embodiments, the risk associated with the pulmonary nodule is the risk of developing a pulmonary or respiratory disease or disorder. In some embodiments, the pulmonary or respiratory disease or disorder is lung cancer, chronic obstructive pulmonary disease (COPD), or a disorder characterized by airflow limitation. In some embodiments, the method identifies or quantifies at least one genetic polymorphism in a biological sample obtained from the subject. In some embodiments, the method entails analyzing the bodily sample to yield a first data set comprising at least one genetic polymorphism in a bodily sample. The genetic polymorphism maybe associated with a first pulmonary or respiratory disease or disorder. In some embodiments, the method comprises analyzing the bodily sample to yield a second data set comprising at least one genetic polymorphism in the bodily sample. In some embodiments, the at least one genetic polymorphism is associated with a second pulmonary or respiratory disease or disorder. In some embodiments, a polymorphism profile is established based on the analysis of the first data set. In some embodiments, a polymorphism profile is established based on the analysis of the second data set. In some embodiments, the method comprises imaging a pulmonary nodule from the subject to determine at least one characteristic associated with said image of the pulmonary nodule. In some embodiments, the method comprises obtaining data with respect to at least one biomarker of the subject. In some embodiments, the method further comprises computer processing the first data set and the second data set to yield a third data set. In some embodiments, the method further comprises analyzing the third data set in conjunction with the at least one characteristic of the pulmonary nodule with the at least one biomarker to determine the risk associated with the pulmonary nodule. In some embodiments, the method comprises electronically outputting a report that identifies the risk presented by the pulmonary nodule. The method may identify two categories of polymorphisms: those associated with a reduced risk of developing lung cancer, which can be termed protective polymorphisms, and those associated with an increased risk of developing lung cancer known as susceptibility polymorphism. In some embodiments, the method comprises assessing a subject's risk of developing a pulmonary or respiratory disease or disorder by determining the presence or absence of at least one protective polymorphism associated with a reduced risk of developing lung cancer and/or the presence or absence of at least one susceptibility polymorphism associated with an increased risk of developing lung cancer. The presence of one or more of said protective polymorphisms may be indicative of a reduced risk of developing lung cancer. The absence of at least one protective polymorphism in combination with the presence of at least one susceptibility polymorphism may be indicative of an increased risk of developing lung cancer. The method may comprise treating a subject for a pulmonary or respiratory disease or disorder. The subject may have been diagnosed with or exhibit at least one symptom associated with the pulmonary or respiratory disease or disorder. The pulmonary or respiratory disease or disorder may be lung cancer. In some embodiments, the method determines an early diagnosis or a therapeutic intervention for treating a subject who is at risk of developing lung cancer or a subject who has already developed lung cancer. In some embodiments, the pulmonary nodule is detected during a procedure or visit related to lung cancer screening. In some embodiments, the pulmonary nodule is detected during a procedure or visit that is not related to lung cancer screening. In some embodiments, the pulmonary nodule is an intermediate risk nodule. In some embodiments, the pulmonary nodule is an indeterminate bodily sample. In some embodiments, the first pulmonary or respiratory disease or disorder is lung cancer. In some embodiments, the second pulmonary or respiratory disease or disorder is lung cancer. In some embodiments, the first pulmonary or respiratory disease or disorder is airflow limitation or chronic obstructive pulmonary disease (COPD). In some embodiments, the second pulmonary or respiratory disease or disorder is airflow limitation or chronic obstructive pulmonary disease (COPD).

In some embodiments, “risk of developing lung cancer” means the likelihood that a subject to will develop lung cancer and include predisposition to and potential onset of lung cancer. In some embodiments, increasing risk of developing lung cancer means that a subject having such an increased risk possesses an hereditary inclination or tendency to develop lung cancer. Subjects with an increased risk of developing lung cancer include those with a predisposition to lung cancer, such as a tendency or predilection regardless of their lung function at the time of assessment, for example, a subject who is genetically inclined to lung cancer but who has normal lung function, those at potential risk, including subjects with a tendency to mildly reduced lung function who are likely to go on to suffer lung cancer if they keep smoking, and subjects with potential onset of lung cancer, who have a tendency to poor lung function on spirometry etc., consistent with lung cancer at the time of assessment.

In some embodiments, decreasing risk of developing lung cancer refers to a subject having such a decreased risk possesses an hereditary disinclination or reduced tendency to develop lung cancer. This docs not mean that such a person will not develop lung cancer at any time, merely that he or she has a decreased likelihood of developing lung cancer compared to the general population of individuals that either docs possess one or more polymorphisms associated with increased lung cancer, or does not possess a polymorphism associated with decreased lung cancer.

### Pulmonary Nodules

Pulmonary nodules may be associated with a particular risk of developing a pulmonary or respiratory disease or disorder. The pulmonary or respiratory disease or disorder may be lung cancer. A pulmonary nodule analyzed may show a higher or lower risk of developing lung cancer. The proportion of screening participants who have pulmonary nodules identified on their annual CT scans varies between about 25% and 50%, depending on the population screened, the definition of a “nodule of interest”, and the methods used for nodule characterization. Examples of methods for nodule characterization may include lung-RADs or volumetric assessment. A nodule of interest may be characterized as being at least about 6 millimeters. However, roughly 96% of all nodules detected during screening are subsequently found not to be lung cancer. While nodule size and radiological (CT) characteristics (often after interval scanning), help to discriminate benign from malignant nodules this remains a complex process. Moreover, a proportion of pulmonary nodules fall into the category of “intermediate risk”, or “indeterminate pulmonary nodule” (IPN). Recognized herein is an industrywide need for improved detection and analysis of pulmonary nodules to characterize the risk associated with the pulmonary nodule. Also recognized herein is a need for improved detection and analysis of intermediate risk nodules, and of recategorizing these as low-risk or high-risk pulmonary nodules.

The present disclosure provides methods for analyzing a pulmonary nodule. In some embodiments, the method comprises analyzing a first data set comprising at lest one genetic polymorphism associated with a first pulmonary respiratory disease or disorder. In some embodiments, the method comprises analyzing a second data set comprising at least one genetic polymorphism associated with a second pulmonary or respiratory disease or disorder. In some embodiments, the first data set comprising the at least one genetic polymorphism is derived from a bodily sample. In some embodiments, the second data set comprising the at least one genetic polymorphism is derived from a bodily sample. The bodily sample may be obtained from, for example, blood, saliva, or buccal swab. In some embodiments, the method comprises obtaining data with respect to at least one biomarker. In some embodiments, the method comprises computer processing the first data set and the second data set to yield a third data set. In some embodiments, the method comprises analyzing the third data set in conjunction with the at least one characteristic of the pulmonary nodule and the at least one biomarker to determine the risk associated with the pulmonary nodule. In some embodiments, the method comprises electronically outputting a report that identifies the risk presented by the pulmonary nodule. In some embodiments, the pulmonary nodule is a low risk pulmonary nodule. In some embodiments, the pulmonary nodule is an intermediate risk pulmonary nodule. In some embodiments, the pulmonary nodule is a high risk pulmonary nodule. The risk associated with a pulmonary nodule could be quantified in terms of the growth rate or malignancy of the pulmonary nodule. In some embodiments, the pulmonary nodule is an indeterminate bodily sample. In some embodiments, the indeterminate bodily sample is blood, serum, buccal swab, tissue sample, nucleic acid sample, or breath condensate. In some embodiments, the pulmonary nodule is detected during a procedure or visit related to lung cancer screening. In some embodiments, the pulmonary nodule is detected during a procedure or visit that is not related to lung cancer screening. In some embodiments, the method comprises imaging a pulmonary nodule from a subject to determine at least one characteristic associated with the image of the pulmonary nodule. Methods as described herein may further comprise analyzing age, gender, family history of lung cancer. CT evidence of emphysema, predicted FEV1, predicted FVC, pack years, total number of nodules, nodule size (either diameter or volume), nodule spiculation, nodule type, nodule location, or nodule lobulation. Pack years may be calculated by multiplying the number of packs of cigarettes smoked per day by the number of years the person has smoked. In some embodiments, the method may further comprise gathering data on one or more subjects in an intermediate risk group following the claimed methods of analysis. In some embodiments, the method may further comprise gathering data on one or more subjects in an intermediate risk group who move to a low-risk group following the claimed methods of analysis. In some embodiments, the method may further comprise gathering data on one or more subjects in an intermediate risk group who move to a higher-risk group following the claimed methods of analysis. In some embodiments, the method may further comprise comparing the risk associated with the pulmonary nodule with data from one or more subjects in an intermediate risk group. In some embodiments, determining the risk associated with the pulmonary nodule may lead to improved clinical outcomes. The improved clinical outcome may be an improvement in Area Under Curve (AUC). The improved clinical outcome may be accurate reclassification of a pulmonary nodule in a subject. The improved clinical outcome may be accurate reclassification of a pulmonary nodule in a subject associated with a pulmonary or respiratory disease or disorder. The improved clinical outcome may be accurate reclassification of an intermediate pulmonary nodule. The reclassification of the intermediate pulmonary nodule may be benign. The reclassification of the intermediate pulmonary nodule may be malign. The improved clinical outcome may be lower surgery rate for a subject with a benign pulmonary nodule. A pulmonary nodule may be benign if associated with lower risk of developing a pulmonary or respiratory disease or disorder, e.g., lung cancer.

Method as described herein may be used for various applications. For example, methods as described herein can be used to characterize incidental nodules, or nodules that are found when scans or x-rays are done for reasons unrelated to any concerns about lung cancer (“incidental” or “non-screen” nodules”).

### Low Risk

Methods as described herein may be used to characterize the risk associated with a pulmonary nodule. Methods as described herein may be used to characterize the risk of developing a pulmonary or respiratory disease or disorder associated with a pulmonary nodule. The pulmonary or respiratory disease or disorder may be lung cancer. The pulmonary or respiratory disease or disorder may be COPD. The risk may be quantified in terms of a polygenic risk score (PRS). A pulmonary nodule may be associated with a low risk of developing a pulmonary or respiratory disease or disorder when the PRS is at least about −10 to at least about −0.1. A pulmonary nodule may be associated with a low risk of developing of a pulmonary or respiratory disease or disorder when the PRS is at least about −10, −9, −8, −7, −6, −5, −4, −3, −2, −1, −0.9, −0.8, −0.7, −0.6, −0.5, −0.4, −0.3, −0.2, −0.1, or more. A pulmonary nodule may be associated with a low risk of developing of a pulmonary or respiratory disease or disorder when the PRS is at least about −4 to at least about −1. In some embodiments, a low risk score is associated with predominantly protective SNPs.

### Intermediate Risk

Methods as described herein may be used to characterize the risk associated with a pulmonary nodule. Methods as described herein may be used to characterize the risk of developing a pulmonary or respiratory disease or disorder associated with a pulmonary nodule. The pulmonary or respiratory disease or disorder may be lung cancer. The pulmonary or respiratory disease or disorder may be COPD. The risk may be quantified in terms of a polygenic risk score (PRS). A pulmonary nodule may be associated with an intermediate risk of developing a pulmonary or respiratory disease or disorder when the PRS is at least about 0) to at least about 1. A pulmonary nodule may be associated with an intermediate risk of developing of a pulmonary or respiratory disease or disorder when the PRS is at least about 0, 0.1, 0.2, 0.3, 0.4, 0.5, 0.6, 0.7, 0.8, 0.9, or more. In some embodiments, an intermediate risk may be associated with equal protective and susceptible SNPs.

### High Risk

Methods as described herein may be used to characterize the risk associated with a pulmonary nodule. Methods as described herein may be used to characterize the risk of developing a pulmonary or respiratory disease or disorder associated with a pulmonary nodule. The pulmonary or respiratory disease or disorder may be lung cancer. The pulmonary or respiratory disease or disorder may be COPD. The risk may be quantified in terms of a polygenic risk score (PRS). A pulmonary nodule may be associated with an intermediate risk of developing a pulmonary or respiratory disease or disorder when the PRS is at least about 1 to at least about 10. A pulmonary nodule may be associated with an intermediate risk of developing of a pulmonary or respiratory disease or disorder when the PRS is at least about 1, 2, 3, 4, 5, 6, 7, 8, 9, 10, or more. A high risk may be associated with predominantly susceptible SNPs.

### Methods Based on SNP Analysis

SNPs based on a patient's germ-line genetic profile may be useful information in determining the risk associated with a pulmonary nodule. The present disclosure provides methods of analyzing single nucleotide polymorphisms (SNPs) to determine the risk of developing a pulmonary or respiratory disease or disorder. In some embodiments, the method comprises analyzing a first data set comprising at lest one genetic polymorphism associated with a first pulmonary respiratory disease or disorder. In some embodiments, the method comprises analyzing a second data set comprising at least one genetic polymorphism associated with a second pulmonary or respiratory disease or disorder. In some embodiments, the first data set comprising the at least one genetic polymorphism is derived from a bodily sample. In some embodiments, the second data set comprising the at least one genetic polymorphism is derived from a bodily sample. The bodily sample may be obtained from, for example, a blood sample, a scrum sample, a plasma sample, a saliva sample, a stool sample, a sputum sample, a urine sample, a semen sample, a transvaginal fluid sample, a cerebrospinal fluid sample, a sweat sample, a cell sample, or a tissue sample. The biomarker may be, for example, proteins, antibodies, or nucleic acids (e.g., liquid or tumor DNA, tumor RNA) obtained from a subject.

In some embodiments, the method comprises obtaining data with respect to at least one biomarker. In some embodiments, the method comprises computer processing the first data set and the second data set to yield a third data set. In some embodiments, the method comprises analyzing the third data set in conjunction with the at least one characteristic of the pulmonary nodule and the at least one biomarker to determine the risk associated with the pulmonary nodule. In some embodiments, the method comprises electronically outputting a report that identifies the risk presented by the pulmonary nodule. In some embodiments, the pulmonary nodule is a low risk pulmonary nodule. In some embodiments, the pulmonary nodule is an intermediate risk pulmonary nodule. In some embodiments, the pulmonary nodule is a high-risk pulmonary nodule. In some embodiments, the pulmonary nodule is an indeterminate bodily sample. In some embodiments, the indeterminate bodily sample is blood, serum, buccal swab, tissue sample, nucleic acid sample, or breath condensate. In some embodiments, the pulmonary nodule is detected during a procedure or visit related to lung cancer screening. In some embodiments, the pulmonary nodule is detected during a procedure or visit that is not related to lung cancer screening. In some embodiments, the method comprises imaging a pulmonary nodule from a subject to determine at least one characteristic associated with the image of the pulmonary nodule. In some embodiments, the method may further comprise gathering data on one or more subjects in an intermediate risk group following the claimed methods of analysis. In some embodiments, the method may further comprise gathering data on one or more subjects in an intermediate risk group who move to a low-risk group following the claimed methods of analysis. In some embodiments, the method may further comprise gathering data on one or more subjects in an intermediate risk group who move to a higher-risk group following the claimed methods of analysis. The method may comprise treating a subject for a pulmonary or respiratory disease or disorder. The subject may have been diagnosed with or exhibit at least one symptom associated with the pulmonary or respiratory disease or disorder. The pulmonary or respiratory disease or disorder may be lung cancer.

Airflow limitation may have an effect on the development of another pulmonary or respiratory disease or disorder. Airflow limitation may have an effect on the development of lung cancer. The presence of airways disease, such as chronic obstructive pulmonary disorder (COPD) may be associated with less diagnosis of patients. The presence of COPD may be associated with more aggressive lung cancer. The presence of COPD may be associated with greater likelihood of death from lung cancer. Airflow limitation may be associated with a lower volume doubling time, or the time a lung cancer takes to double in size. This increase in size may be a marker of greater malignant potential. Forced Expiratory Volume (FEV) is the volume of air that can be exhaled during a forced breath in t seconds. FEV1 is the amount of air that can be forced during a breath in one second. FVC is the maximum amount of air that can be inhaled after maximum exhalation. Reduced FEV1 or reduced FVC may be features of obstructed and/or restricted spirometry. Reduced FEV1 or reduced FVC may be associated with a greater tendency to nodule malignancy or development of cancer. The present disclosure provides methods for analyzing SNPs to determine the risk associated with a pulmonary nodule. In some embodiments, the SNP may be associated with a pulmonary or respiratory disease or disorder. In some embodiments, the pulmonary or respiratory disease or disorder may be lung cancer. In some embodiments, the pulmonary or respiratory disease or disorder may be airflow limitation. In some embodiments, the pulmonary or respiratory disease or disorder may be COPD. In some embodiments, methods as described herein comprise combining SNP variants that increase or decrease the risk of having a first pulmonary or respiratory disease or disorder with SNP variants that increase or decrease the risk of developing a second pulmonary or respiratory disease or disorder. In some embodiments, methods as described herein comprise combining SNP variants that increase or decrease the risk of having airflow limitation with SNP variants that increase or decrease the risk of developing lung cancer. The risk associated with a pulmonary nodule could be quantified in terms of the growth rate or malignancy of the pulmonary nodule. In some embodiments, methods as described herein further comprise analyzing or determining whether a pulmonary nodule has greater malignant potential. Methods as described herein may further comprise analyzing age, gender, family history of lung cancer. CT evidence of emphysema, predicted FEV1, predicted FVC, pack years, total number of nodules, nodule size (either diameter or volume), nodule spiculation, nodule type, nodule location, or nodule lobulation. Pack years may be calculated by multiplying the number of packs of cigarettes smoked per day by the number of years the person has smoked.

The present disclosure provides methods of determining a polygenic risk score associated with a pulmonary nodule. In some embodiments, the polygenic risk score is calculated by determining a first set of SNPs that are susceptibility SNPs and a second set of SNPs that are protective SNPs. In some embodiments, a susceptibility SNP is a SNP that is related with higher risk of developing a disease or disorder. In some embodiments, the disease or disorder is a pulmonary or respiratory disease or disorder. In some embodiments, the protective SNP is a SNP related to lower risk of developing a disease or disorder. In some embodiments, the disease or disorder is a pulmonary or respiratory disease or disorder. In some embodiments, the polygenic risk score is further calculated by determining the net genetic risk score. The net genetic risk score may be found by adding the number of susceptibility SNPs and subtracting the number of protective SNPs. Examples of susceptible and protective SNPs are provided in Table 1. Examples of SNP may include any SNP in full linkage disequilibrium to one or more SNPs as disclosed herein. In some embodiments, the polygenic risk score is calculated by adding the net genetic risk score to other clinical variables. Various clinical risk models may be used. An example of a clinical risk model is the BROCK model published by McWilliams and colleagues. Methods as described herein may further comprise analyzing age, gender, family history of lung cancer. CT evidence of emphysema, predicted FEV1, predicted FVC, pack years, total number of nodules, nodule size (either diameter or volume), nodule spiculation, nodule type, nodule location, or nodule lobulation. Pack years may be calculated by multiplying the number of packs of cigarettes smoked per day by the number of years the person has smoked. In some embodiments, determining the risk associated with the pulmonary nodule may lead to improved clinical outcomes. The improved clinical outcome may be an improvement in Area Under Curve (AUC). The improved clinical outcome may be accurate reclassification of a pulmonary nodule in a subject. The improved clinical outcome may be accurate reclassification of a pulmonary nodule in a subject associated with a pulmonary or respiratory disease or disorder. The improved clinical outcome may be accurate reclassification of an intermediate pulmonary nodule. The reclassification of the intermediate pulmonary nodule may be benign. The reclassification of the intermediate pulmonary nodule may be malign. The improved clinical outcome may be lower surgery rate for a subject with a benign pulmonary nodule. A pulmonary nodule may be benign if associated with lower risk of developing a pulmonary or respiratory disease or disorder, e.g., lung cancer.

TABLE 1
List of germline SNPs associated with lung function
and lung cancer risk that may contribute to determining
the malignant potential of a lung nodule.

| Gene name | | Effect on nodule |
|---|---|---|
| (rs number) | Genotype | malignancy risk |
| HHIP (rs1489759) | GG (protective) | reduced |
| GYPA (rs2202507) | GG (protective) | reduced |
| FAM13A (rs 7671167) | CC (protective) | reduced |
| HTR4 (rs 11168048) | CC (protective) | reduced |
| DLC1 (rs58863591) | CT/TT | increased |
| | (susceptible) | |
| RIN3 (rs754388) | CG/GG | reduced |
| | (protective) | |
| HHIP (rs13141641) | CC (protective) | reduced |
| ADAM19 (rs1422795) | CC (susceptible) | increased |
| CHRNA5 (rs16969968) | AA (susceptible) | increased |
| TERT (rs402710) | CC (susceptible) | increased |
| IREB (rs2656069) | CC/CT | reduced |
| | (protective) | |
| CYP2A6 (rs7937) | TT (susceptible) | increased |
| BAT3 (rs1052486) | GG (susceptible) | increased |
| AGER (rs2070600) | TT/TC | ?increased/?reduced |
| | (susceptible) | |
| ITGA11 (rs2306022) | TT/TC | reduced |
| | (protective) | |
| CRP (rs2808630) | CC (protective) | reduced |
| MMP12 (rs645419) | GG (protective) | reduced |
| FasL (rs763110) | TT (protective) | reduced |
| Rev1 (rs3087386) | GG (protective) | reduced |
| IL-6 (rs1800797) | GG (susceptible) | increased |

The present disclosure provides methods for analyzing clinical and nodule variables to determine the risk associated with a pulmonary nodule. In some embodiments, the risk associated with a pulmonary nodule is the risk of developing a pulmonary or respiratory disease or disorder. In some embodiments, the risk associated with a pulmonary nodule is the risk of developing lung cancer. Examples of clinical and nodule variables may be found in Table 2. The method of analyzing clinical and nodule variables may comprise analyzing age, gender, family history of lung cancer, CT evidence of emphysema, predicted FEV1, predicted FVC, pack years, total number of nodules, nodule size (either diameter or volume), nodule spiculation, nodule type, nodule location, or nodule lobulation. Pack years may be calculated by multiplying the number of packs of cigarettes smoked per day by the number of years the person has smoked.

TABLE 2
Clinical and nodule variables that effect the risk of
malignancy of a screen-detected nodule.

| Patient | | Effect on risk of | |
|---|---|---|---|
| Variable | Characteristic | malignancy | Other |
| | Age | ↑ risk with ↑ age | |
| | gender | ↑ risk with men | |
| | Family history (FHx) | ↑ risk with positive FHx | |
| | of lung cancer | | |
| | CT evidence of | ↑ risk with emphysema | |
| | emphysema | | |
| | FEV1% predicted | ↑ risk with ↓ FEV1% | |
| | FVC % predicted | ↑ risk with ↓ FVC% | |
| | Pack years | ↑ risk with ↑ pack years | |
| Nodule | Total number of nodules | ↑ risk with ↑ total nodules | |
| | Nodule size | ↑ risk with ↑ nodule size | |
| | (diameter or volume) | | |
| | Nodule spiculation | ↑ risk with nodule | |
| | | spiculation | |
| | Nodule type | ↑ risk with part solid | |
| | Nodule location | ↑ risk with upper lobe | |
| | Nodule lobulation | ↑ risk with lobulation | |

Described herein, in some embodiments, are methods for analyzing at least genetic polymorphism for determining the risk associated with a pulmonary nodule. In some embodiments, the risk associated with a pulmonary nodule is the risk of developing a pulmonary or respiratory disease or disorder. In some embodiments, the risk associated with a pulmonary nodule is the risk of developing lung cancer. In some embodiments, the method identifies at least one genetic polymorphism in a biological sample from said subject in need thereof to obtain a polymorphism profile of said subject. In some embodiments, the method quantifies at least one genetic polymorphism in a biological sample from said subject in need thereof to obtain a polymorphism profile of said subject. In some embodiments, the method identifies and quantifies at least one genetic polymorphism in a biological sample from said subject in need thereof to obtain a polymorphism profile of said subject. In some embodiments, the method takes into consideration said polymorphism profile, a level (e.g., probability, odds ratio, p value, etc.) of said lung cancer-associated risk of said subject with a sensitivity or a specificity determined according to a receiver operator characteristic (ROC) curve having an area under curve (AUC) value with a confidence interval. In some embodiments, the AUC value determined from methods described herein is at least or equal to about 0.50, 0.51, 0.52, 0.53, 0.54, 0.55, 0.56, 0.57, 0.58, 0.59, 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.88, 0.99, or higher. In some embodiments, the AUC value determined from the methods described herein is increased by at least or equal to about 1.0%, 2.0%, 3.0%, 4.0%, 5.0%, 10.0%, 15.0%, 20.0%, 25.0%, 30.0%, 35.0%, 40.0%, 45.0%, 50.0%, 55.0%, 60.0%, 65.0%, 70.0%, 75.0%, 80.0%, 85.0%, 90.0%, 95.0%, 99.0%, or more compared to an AUC value determined by a corresponding performance rate of a corresponding determination without identifying or/and quantifying said at least one genetic polymorphism. In some embodiments, the confidence interval determined by the methods described herein is at least or equal to 60.0%, 65.0%, 70.0%, 75.0%, 80.0%, 85.0%, 90.0%, 91.0%, 92.0%, 93.0%, 94.0%, 95.0%, 96.0%, 97.0%, 98.0%, 99.0%, 99.5%, or 99.9%. In some embodiments, the confidence interval determined by the methods described herein is increased by at least or equal to about 1.0%, 2.0%, 3.0%, 4.0%, 5.0%, 10.0%, 15.0%, 20.0%, 25.0%, 30.0%, 35.0%, 40.0%, 45.0%, 50.0%, 55.0%, 60.0%, 65.0%, 70.0%, 75.0%, 80.0%, 85.0%, 90.0%, 95.0%, 99.0%, or more compared to a confidence interval as determined by corresponding performance rate of a corresponding determination without identifying or/and quantifying said at least one genetic polymorphism. The risk associated with a pulmonary nodule could be quantified in terms of the growth rate or malignancy of said pulmonary nodule.

Also described herein, in some embodiments, are methods for determining a lung cancer-associated risk of a subject, comprising: identifying or quantifying at least one genetic polymorphism in a biological sample from said subject in need thereof to obtain a polymorphism profile of said subject; and determining, taking into consideration said polymorphism profile, a level (e.g., probability, odds ratio, p value, etc.) of said lung cancer-associated risk of said subject with an increased sensitivity or an increased specificity. In some embodiments, the method increases the sensitivity by at least or equal to about of at least about 1%, 2%, 5%, 10%, 15%, 20%, 25%, 30%, 35%, 40%, 45%, 50%, 55%, 60%, 65%, 70%, 75%, 80%, 85%, 90%, 95%, 99%, or higher compared to a sensitivity of the corresponding performance rate of a corresponding determination without identifying or/and quantifying said at least one genetic polymorphism. In some embodiments, the method increases the specificity by at least or equal to about of at least about 1%, 2%, 5%, 10%, 15%, 20%, 25%, 30%, 35%, 40%, 45%, 50%, 55%, 60%, 65%, 70%, 75%, 80%, 85%, 90%, 95%, 99%, or higher compared to a specificity of the corresponding performance rate of a corresponding determination without identifying or/and quantifying said at least one genetic polymorphism.

In some embodiments, the method increases the true positive rate by at least or equal to about of at least about 1%, 2%, 5%, 10%, 15%, 20%, 25%, 30%, 35%, 40%, 45%, 50%, 55%, 60%, 65%, 70%, 75%, 80%, 85%, 90%, 95%, 99%, or higher compared to a true positive rate of the corresponding performance rate of a corresponding determination without identifying or/and quantifying said at least one genetic polymorphism. In some embodiments, the method decreases the false positive rate by at least or equal to about of at least about 1%, 2%, 5%, 10%, 15%, 20%, 25%, 30%, 35%, 40%, 45%, 50%, 55%, 60%, 65%, 70%, 75%, 80%, 85%, 90%, 95%, 99%, or higher compared to a false positive rate of the corresponding performance rate of a corresponding determination without identifying or/and quantifying said at least one genetic polymorphism.

In some embodiments, the method increases the true negative rate by at least or equal to about of at least about 1%, 2%, 5%, 10%, 15%, 20%, 25%, 30%, 35%, 40%, 45%, 50%, 55%, 60%, 65%, 70%, 75%, 80%, 85%, 90%, 95%, 99%, or higher compared to a true negative rate of the corresponding performance rate of a corresponding determination without identifying or/and quantifying said at least one genetic polymorphism. In some embodiments, the method decreases the false negative rate by at least or equal to about of at least about 1%, 2%, 5%, 10%, 15%, 20%, 25%, 30%, 35%, 40%, 45%, 50%, 55%, 60%, 65%, 70%, 75%, 80%, 85%, 90%, 95%, 99%, or higher compared to a false negative rate of the corresponding performance rate of a corresponding determination without identifying or/and quantifying said at least one genetic polymorphism.

In some embodiments, the risk of developing or dying from lung cancer is at least partially determined by a PLCO_{M2012} score described herein. In some embodiments, the risk of developing or dying from lung cancer is at least or equal to about 0.1%, 0.5%, 1.0%, 2.0%, 3.0%, 4.0%, 5.0%, 6.0%, 7.0%, 8.0%, 9.0%, 10.0%, 11.0%, 12.0%, 13.0%, 14.0%, 15.0%, 16.0%, 17.0%, 18.0%, 19.0%, or 20.0%.

The present disclosure provides methods of processing or analyzing a bodily sample in a subject. The bodily sample may be selected from the group consisting of: a blood sample, a serum sample, a plasma sample, a saliva sample, a stool sample, a sputum sample, a urine sample, a semen sample, a transvaginal fluid sample, a cerebrospinal fluid sample, a sweat sample, a cell sample, and a tissue sample. The method may comprise analyzing the bodily sample to yield a first data set comprising at least one genetic polymorphism associated with a first pulmonary or respiratory disease or disorder. The method may further comprise analyzing a second data set comprising at least one genetic polymorphism associated with a second pulmonary or respiratory disease or disorder. The at least one genetic polymorphism of the first data set or the second data set may be associated with a risk of pulmonary or respiratory disease or disorder. The pulmonary or respiratory disease or disorder may be lung cancer. The pulmonary or respiratory disease or disorder may be airflow limitation. The pulmonary or respiratory disease or disorder may be COPD. Method as described herein may further comprise imaging a pulmonary nodule from the subject to determine at least one characteristic associated with the image of said pulmonary nodule. Methods as described herein may further comprise obtaining data with respect to at least one biomarker of the subject. The biomarker may be, for example, proteins, antibodies, or nucleic acids (e.g., liquid or tumor DNA, tumor RNA) obtained from a subject. Method as described herein may further comprise computer processing the first data set and the second data set to produce a third data set, and further analyzing the third data set and said at least one characteristic of the pulmonary nodule. Methods as described herein may further comprise determining the risk associated with the pulmonary nodule to an accuracy of at least about 80%. The accuracy may be a percentage of a plurality of independent test samples corresponding to (1) a first plurality of subjects having a pulmonary nodule exhibiting malignancy to (2) a second plurality of subjects having a pulmonary nodule not exhibiting malignancy. The accuracy may be a percentage of a plurality of independent test samples corresponding to (1) a first plurality of subjects receiving an elevated risk-value of said pulmonary or respiratory disease or disorder, relative to (2) a second plurality of subjects having said pulmonary or respiratory disease or disorder that is correctly determined to have or be at risk of said pulmonary or respiratory disease or disorder. The risk associated with a pulmonary nodule could be quantified in terms of the growth rate or malignancy of said pulmonary nodule.

Methods as described herein may further comprise electronically outputting a report that identifies the risk presented by the pulmonary nodule. The pulmonary nodule may be detected during a procedure or a visit that is related to lung cancer screening. The pulmonary nodule may be detected during a procedure or a visit that is not related to lung cancer screening. In some embodiments, the pulmonary nodule is an intermediate risk nodule. In some embodiments, the pulmonary nodule is an indeterminate bodily sample. In some embodiments, the first pulmonary or respiratory disease or disorder is lung cancer. In some embodiments, the second pulmonary or respiratory disease or disorder is lung cancer. In some embodiments, the first pulmonary or respiratory disease or disorder is airflow limitation or chronic obstructive pulmonary disease (COPD). In some embodiments, the second pulmonary or respiratory disease or disorder is airflow limitation or chronic obstructive pulmonary disease (COPD).

In some embodiments, the method further determines a preventive intervention for said subject. In some embodiments, the method further determines a therapeutic intervention (e.g., effective against lung cancer) for said subject. In some embodiments, the method determines the early diagnosis or therapeutic intervention based on the analysis of the polymorphism profile described herein. In some embodiments, the method determines the early diagnosis or therapeutic intervention based on the analysis of the at least one clinical variable described herein. In some embodiments, the method determines the early diagnosis or therapeutic intervention based on the analysis of the polymorphism profile and the at least one clinical variable described herein.

In some embodiments, the polymorphism profile comprises at least one genomic locus, genomic location, or SNP described herein. The genomic locus, genomic location, or SNP described herein may be categorized as “susceptible” for increasing the susceptibility of the subject's risk of developing lung cancer. In an alternative, the genomic locus, genomic location, or SNP described herein may be categorized as “protective” for protecting or decreasing the susceptibility of the subject's risk of developing lung cancer. The genomic locus, genomic location, or SNP described herein may be categorized as “susceptible” for the subject's risk of developing COPD. In some embodiments, the genomic locus, genomic location, or SNP described herein may be categorized as “protective” for the subject's risk of developing COPD. In some embodiments, the at least one genomic locus, genomic location, or SNP belongs to the coding or non-coding region of any one of the lung cancer-associated gene described herein. Non-limiting examples of the lung cancer-associated gene include Cholinergic receptor nicotinic alpha 3 subunit (CHRNA3), Cholinergic receptor nicotinic alpha 5 subunit (CHRNA5), Telomerase reverse transcriptase (TERT), HLA-B associated transcript 3 (BAT3), Cytochrome P450 family 2 subfamily A member 6 (CYP2A6), Iron responsive element binding protein 2 (IREB), Fas cell surface death receptor ligand (FasL), REV1 DNA directed polymerase (Rev1), Integrin subunit alpha 11 (ITGA11). Matrix metallopeptidase 12 (MMP12), Interleukin 6 (IL-6), C-Reactive protein (CRP), or Advanced glycosylation end-product specific receptor (AGER). In some embodiments, the lung cancer-associated gene can be any one of the gene selected from Table 8 (e.g., CHRNA5, CYP2E1, IL-18, IL-8, IL-1B, ITGA11, NAT-2, 1-ACT, Cerberus 1 (Cer1), DAT1, TNFR1, TLR9, P73, SOD3, ITGB3, DRD2, BCL2, XPD (ERCC2), Rev1, FasL, BAT3, TERT, CRP, FAM13A, HHIP, ADAM19, AGER, GYPA, GRP126, GSTCD, IREB, HTR4, RIN3, ADCY2, MMP12, CYP2A6, DLC1, IL-6).

In some embodiments, the at least one genetic polymorphism comprises one or more (e.g., 12 or more, etc.) single nucleotide polymorphisms (SNPs) selected from: a polymorphism (e.g., GG genotype) at rs1489759 of human Hedgehog interacting protein (HHIP) gene: a polymorphism (e.g., CC genotype) at rs13141641 of human Hedgehog interacting protein (HHIP) gene: a polymorphism (e.g., GG or/and CC genotype) at rs2202507 of glycophorin A (GYPA) gene: a polymorphism (e.g., CC genotype) at rs7671167 of family with sequence similarity 13 member A (FAM13A) gene: a polymorphism (e.g., CG or GG genotype) at rs754388 of Ras and Rab interactor 3 (RIN3) gene: a polymorphism (e.g., CC genotype) at rs11168048 of 5-hydroxytryptamine receptor 4 (HTR4) gene: a polymorphism (e.g., CT, or TT genotype) at rs58863591 of deleted in liver cancer 1 (DLC1) gene: a polymorphism (e.g., CC genotype) at rs1422795 in a disintegrin and metalloproteinase 19 (ADAM19) gene: a polymorphism (e.g., TT or TC genotype) at rs2306022 integrin alpha 11 (ITGA11) gene: a polymorphism (e.g., CC genotype) at rs2808630 of C-reactive protein (CRP) gene: a polymorphism (e.g., GG genotype) at rs3087386 of DNA repair protein (Rev1) gene: a polymorphism (e.g., GG genotype) at rs645419 of matrix metalloproteinase-12 (MMP12) gene: a polymorphism (e.g., TT genotype) at rs763110 of Fas ligand (FasL) gene: a polymorphism (e.g., GG genotype) at rs1052486 of HLA-B-associated transcript 3 (BAT3) gene: a polymorphism (e.g., GG genotype) at rs1800797 of interleukin-6 (IL-6) gene: a polymorphism (e.g., TT or TC genotype) at rs2070600 of advanced glycosylation end product-specific receptor (AGER) gene: a polymorphism (e.g., CC or CT genotype) at rs2656069 of iron regulatory protein (IREB) gene: a polymorphism (e.g., AA genotype) at rs16969968 of cholinergic receptor nicotinic alpha 3/5 (CHRNA3/5) gene: a polymorphism (e.g., CC genotype) at rs402710 of telomerase reverse transcriptase (TERT) gene; or a polymorphism (e.g., TT genotype) at rs7937 of cytochrome P450 family 2 subfamily A member 6 (CYP2A6) gene. In some embodiments, the HHIP is, for example, an HHIP-1 polymorphism or an HHIP-2 polymorphism.

In some embodiments, the at least one genetic polymorphism comprises one or more single nucleotide polymorphisms (SNPs) described herein. In some embodiments, the at least one genetic polymorphism comprises at least one chronic obstructive pulmonary disease (COPD)-protective SNP. In some embodiments, the at least one genetic polymorphism comprises at least one COPD-susceptible SNP. In some embodiments, the at least one genetic polymorphism comprises at least one lung cancer-protective SNP. In some embodiments, the at least one genetic polymorphism comprises at least one lung cancer-susceptible SNP. In some embodiments, the at least one genetic polymorphism comprises at least one lung cancer and COPD dual protective SNP. In some embodiments, the at least one genetic polymorphism comprises at least one lung cancer and COPD dual susceptible SNP.

In some embodiments, the at least one COPD-protective SNP comprises one or more SNPs selected from: a polymorphism in human Hedgehog interacting protein (HHIP) gene (e.g., at rs1489759 (e.g., GG)), a polymorphism in human Hedgehog interacting protein (HHIP) gene (e.g., at rs13141641 (e.g., CC)), a polymorphism in glycophorin A (GYPA) gene (e.g., at rs2202507 (e.g., GG)), a polymorphism in family with sequence similarity 13 member A (FAM13A) gene (e.g., at rs7671167 (e.g., CC)), a polymorphism in Ras and Rab interactor 3 (RIN3) gene (e.g., at rs754388 (e.g., CG or/and GG)), and a polymorphism in 5-hydroxytryptamine receptor 4 (HTR4) gene (e.g., at rs11168048 (e.g., CC)). In some embodiments, the HHIP is, for example, an HHIP-1 polymorphism or an HHIP-2 polymorphism.

In some embodiments, the at least one COPD-susceptible SNP of (ii) comprises one or more SNPs selected from: a polymorphism in deleted in liver cancer 1 (DLC1) gene (e.g., at rs58863591 (e.g., CT or/and TT)), and a polymorphism in a disintegrin and metalloproteinase 19 (ADAM19) gene (e.g., at rs1422795 (e.g., CC)).

In some embodiments, the at least one lung cancer-protective SNP comprises one or more SNPs selected from: a polymorphism in integrin alpha 11 (ITGA11) gene (e.g., at rs2306022 (e.g., TT or/and TC)), a polymorphism in C-reactive protein (CRP) gene (e.g., at rs2808630 (e.g., CC)), a polymorphism in DNA repair protein (Rev1) gene (e.g., at rs3087386 (e.g., GG)), a polymorphism in matrix metalloproteinase-12 (MMP12) gene (e.g., at rs645419 (e.g., GG)), and a polymorphism in Fas ligand (FasL) gene (e.g., at rs763110 (e.g., TT)). In some embodiments, the at least one lung cancer-susceptible SNP of comprises one or more SNPs selected from: a polymorphism in HLA-B-associated transcript 3 (BAT3) gene (e.g., at rs1052486 (e.g., GG)), and a polymorphism in interleukin-6 (IL-6) gene (e.g., at rs1800797 (e.g., GG)). In some embodiments, the at least one lung cancer-susceptible, COPD-protective SNP comprises a polymorphism in advanced glycosylation end product-specific receptor (AGER) gene (e.g., at rs2070600 (e.g., TT or/and TC)). In some embodiments, the at least one lung cancer and COPD dual protective SNP m of (vi) comprises a polymorphism in iron regulatory protein (IREB) gene (e.g., at rs2656069 (e.g., CC or/and CT)). In some embodiments, the at least one lung cancer and COPD dual susceptible SNP of (vii) comprises one or more SNPs selected from: a polymorphism in cholinergic receptor nicotinic alpha 3/5 (CHRNA3/5) gene (e.g., at rs16969968 (e.g., AA)), a polymorphism in telomerase reverse transcriptase (TERT) gene (e.g., at rs402710 (e.g., CC)), a polymorphism in cytochrome P450 family 2 subfamily A member 6 (CYP2A6) gene (e.g., at rs7937 (e.g., TT)). In some embodiments, the polymorphism profile indicates a genetic predisposition (e.g., predominantly protective, predominantly susceptible, or neutral) of said subject to a lung cancer-associated condition (e.g., developing lung cancer (e.g., with or without accompanying COPD) or dying of lung cancer).

In some embodiments, the method determines the lung cancer-associated risk at least partially dependent on analyzing the one or more members selected from: incidence, onset, stage, progression, mortality, survival, subtype, and presence or absence of an accompanying comorbidity (e.g., within a period of time). In some embodiments, the lung cancer-associated risk comprises a likelihood that a subject is predisposed to develop or die of lung cancer within a period of time (e.g., about 5 years). In some embodiments, a level of the risk of dying of lung cancer (e.g., within 5 years) is at least about 0.5%, 0.6%, 0.7%, 0.9%, 0.8%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.5%, 5.0%, 5.5%, 6.0%, 7.0%, 8.0%, 9.0%, 10.0%, 15.0%, 20.0%, or a higher level. In some embodiments, the determination of the lung cancer-associated risk comprises a likelihood that a subject is predisposed to develop lung cancer with or without accompanying COPD within a period of time

### Subject

Described herein, in some embodiments, are methods for determining a lung cancer-associated risk of a subject. In some embodiments, the subject has not been diagnosed with COPD. In some embodiments, the subject has been diagnosed with (e.g., mild-moderate) COPD. In some embodiments, the method determines a lung cancer-associated risk for a subject regardless of whether said subject has been diagnosed with COPD. In some embodiments, the subject is determined as having an intermediate risk of developing or dying of lung cancer. In some embodiments, the risk of developing or dying from lung cancer is at least partially determined by a PLCOM2012 score described herein. In some embodiments, the risk of developing or dying from lung cancer is at least or equal to about 0.1%, 0.5%, 1.0%, 2.0%, 3.0%, 4.0%, 5.0%, 6.0%, 7.0%, 8.0%, 9.0%, 10.0%, 11.0%, 12.0%, 13.0%, 14.0%, 15.0%, 16.0%, 17.0%, 18.0%, 19.0%, or 20.0%.

In some embodiments, the method determines a risk of developing or dying pf lung cancer comprises determining at least one clinical variable of a subject. In some embodiments, the clinical variable includes the subject's exposure to cigarette smoking. In some embodiments, the subject may be actively or passively exposed to smoking (e.g., tobacco smoking, such as cigarette smoking, pipe smoking, cigar smoking, use of chewing tobacco, or a combination thereof). In some embodiments, the subject is an active tobacco consumer or an active smoker (e.g., having a smoking status of at least 2 cigarettes per day). Additional clinical variables that can be determined for the subject's risk of developing or dying of lung cancer include the subject's age, gender, self-reported comorbidity, spirometric-defined comorbidity, history of chronic obstructive pulmonary disease (COPD), family history of lung cancer, body mass index (BMI), lung function, lung cancer histology, lung cancer stage, or surgical history.

### Assaying Biological Samples

The biological samples may be obtained or derived from a subject (e.g., a human subject). The biological samples may be stored in a variety of storage conditions before processing, such as different temperatures (e.g., at room temperature, under refrigeration or freezer conditions, at 25° C., at 4° C., at −18° C., −20° C., or at −80° C.) or different suspensions (e.g., EDTA collection tubes, the RNA collection tubes, or DNA collection tubes). In some embodiments, the biological sample is a cell-free biological sample. The biological sample may be obtained from cerebrospinal fluid (CSF), saliva, serum, plasma, urine, buccal swab, nasal swab, liquid biopsy, or a combination thereof, from said subject.

The biological sample may be obtained from a subject who does not have lung cancer, from a subject who is at risk of developing lung cancer, from a subject who is suspected of having lung cancer, a subject who has already developed lung cancer, or from a subject who exhibits at least one of the clinical variables described herein. Non-limiting examples of lung cancer can include non-small cell lung cancer (NSCLC), small cell lung cancer (SCLC), or any other lung cancer type, including adenocarcinoma, squamous carcinoma, large cell (undifferentiated) carcinoma, large cell neuroendocrine carcinoma, adenosquamous carcinoma, sarcomatoid carcinoma, lung carcinoid tumor, or adenoid cystic carcinoma. Other exemplary lung cancer may include lymphoma, sarcoma, benign lung tumor, or hamartoma.

The biological sample may be taken before and/or after treatment of a subject with lung cancer. The biological samples may be obtained from a subject during a treatment or a treatment regime. Multiple biological samples may be obtained from a subject to monitor the effects of the treatment over time. The biological sample may be taken from a subject known or suspected of having lung cancer for which a definitive positive or negative diagnosis is not available via clinical tests. The biological sample may be taken from a subject suspected of having lung cancer. The biological sample may be taken from a subject experiencing unexplained symptoms, such as fatigue, nausea, weight loss, aches and pains, weakness, or bleeding. The biological sample may be taken from a subject having explained symptoms. The biological sample may be taken from a subject at risk of developing lung cancer due to any one of or any combination of the clinical variables described herein.

The biological sample may contain one or more analytes capable of being assayed, such as the ribonucleic acid (cfRNA) molecules suitable for assaying to generate transcriptomic data, the deoxyribonucleic acid (cfDNA) molecules suitable for assaying to generate genomic or sequencing data (e.g., SNP data), proteins suitable for assaying to generate proteomic data, metabolites suitable for assaying to generate metabolomic data, or a mixture or combination thereof. One or more such analytes (e.g., cfRNA molecules, cfDNA molecules, proteins, or metabolites) may be isolated or extracted from one or more the biological samples of a subject for downstream assaying using one or more suitable assays.

After obtaining the biological sample from the subject, the biological sample may be processed to generate datasets for determining a lung cancer-associated risk of the subject. For example, a presence, absence, or quantitative assessment of nucleic acid molecules of the biological sample at a panel of lung cancer-associated genomic loci or SNP (e.g., quantitative measures of RNA transcripts or DNA at the lung cancer-associated genomic loci or SNP), proteomic data comprising quantitative measures of proteins of the dataset at a panel of lung cancer-associated proteins, methylation assays, telomere length assays, and/or metabolome data comprising quantitative measures of a panel of lung cancer-associated metabolites may be indicative of lung cancer. Processing the biological sample obtained from the subject may: subject the biological sample to conditions that are sufficient to isolate, enrich, or extract a plurality of nucleic acid molecules, proteins, and/or metabolites; and assay the plurality of nucleic acid molecules, proteins, and/or metabolites to generate the dataset.

In some embodiments, a plurality of nucleic acid molecules is extracted from the biological sample and subjected to sequencing to generate a plurality of sequencing reads. The nucleic acid molecules may comprise ribonucleic acid (RNA) or deoxyribonucleic acid (DNA). The nucleic acid molecules (e.g., RNA or DNA) may be extracted from the biological sample by a variety of methods, such as a FastDNA Kit protocol from MP Biomedicals, a QIAamp DNA the biological mini kit from Qiagen, or a biological DNA isolation kit protocol from Norgen Biotek. The extraction method may extract all RNA or DNA molecules from a sample. Alternatively, the extract method may selectively extract a portion of RNA or DNA molecules from a sample. Extracted RNA molecules from a sample may be converted to DNA molecules by reverse transcription (RT).

The sequencing may be performed by any suitable sequencing methods, such as massively parallel sequencing (MPS), paired-end sequencing, high-throughput sequencing, next-generation sequencing (NGS), shotgun sequencing, single-molecule sequencing, nanopore sequencing, semiconductor sequencing, pyrosequencing, sequencing-by-synthesis (SBS), sequencing-by-ligation, sequencing-by-hybridization, and RNA-Seq (Illumina).

The sequencing may comprise nucleic acid amplification (e.g., of RNA or DNA molecules). In some embodiments, the nucleic acid amplification is polymerase chain reaction (PCR). A suitable number of rounds of PCR (e.g., PCR, qPCR, reverse-transcriptase PCR, digital PCR, etc.) may be performed to sufficiently amplify an initial amount of nucleic acid (e.g., RNA or DNA) to a desired input quantity for subsequent sequencing. In some cases, the PCR may be used for global amplification of target nucleic acids. This may comprise using adapter sequences that may be first ligated to different molecules followed by PCR amplification using universal primers. PCR may be performed using any of a number of commercial kits, e.g., provided by Life Technologies. Affymetrix, Promega, Qiagen, etc. In other cases, only certain target nucleic acids within a population of nucleic acids may be amplified. Specific primers, possibly in conjunction with adapter ligation, may be used to selectively amplify certain targets for downstream sequencing. The PCR may comprise targeted amplification of one or more genomic loci, such as genomic loci associated with lung cancers. The sequencing may comprise use of simultaneous reverse transcription (RT) and polymerase chain reaction (PCR), such as a OneStep RT-PCR kit protocol by Qiagen, NEB, Thermo Fisher Scientific, or Bio-Rad.

RNA or DNA molecules isolated or extracted from the biological sample may be tagged, e.g., with identifiable tags, to allow for multiplexing of a plurality of samples. Any number of RNA or DNA samples may be multiplexed. For example a multiplexed reaction may contain RNA or DNA from at least about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, or more than 100 of the biological samples. For example, a plurality of the biological samples may be tagged with sample barcodes such that each DNA molecule may be traced back to the sample (and the subject) from which the DNA molecule originated. Such tags may be attached to RNA or DNA molecules by ligation or by PCR amplification with primers.

After subjecting the nucleic acid molecules to sequencing, suitable bioinformatics processes may be performed on the sequence reads to generate the data indicative of the presence, absence, or relative assessment of the lung cancer. For example, the sequence reads may be aligned to one or more reference genomes (e.g., a genome of one or more species such as a human genome). The aligned sequence reads may be quantified at one or more genomic loci to generate the datasets indicative of the lung cancer. For example, quantification of sequences corresponding to a plurality of genomic loci associated with lung cancers may generate the datasets indicative of the lung cancer.

The biological sample may be processed without any nucleic acid extraction. For example, lung cancer may be identified or monitored in the subject by using probes configured to selectively enrich nucleic acid (e.g., RNA or DNA) molecules corresponding to the plurality of lung cancer-associated genomic or SNP loci. The probes may be nucleic acid primers. The probes may have sequence complementarity with nucleic acid sequences from one or more of the plurality of lung cancer-associated genomic loci, genomic regions, or SNPs. The plurality of lung cancer-associated genomic loci, genomic regions, or SNPs may comprise at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, or more distinct lung cancer-associated genomic loci, genomic regions, or SNPs. The plurality of lung cancer-associated genomic loci, genomic regions, or SNPs may comprise one or more members (e.g., 1, 2, 3, 4, 5, 6, 7, 8, 9, 10, 11, 12, 13, 14, 15, 16, 17, 18, 19, 20, about 25, about 30, about 35, about 40, about 45, about 50, about 55, about 60, about 65, about 70, about 75, about 80, or more) of the polymorphisms described herein. The lung cancer-associated genomic loci, genomic regions, or SNPs may be associated with at least one of the clinical variables described herein.

The probes may be nucleic acid molecules (e.g., RNA or DNA) having sequence complementarity with nucleic acid sequences (e.g., RNA or DNA) of the one or more genomic loci (e.g., lung cancer-associated genomic loci). These nucleic acid molecules may be primers or enrichment sequences. The assaying of the biological sample using probes that are selective for the one or more genomic loci (e.g., lung cancer-associated genomic loci) may comprise use of array hybridization (e.g., microarray-based), polymerase chain reaction (PCR), methylation detection, telomere length assays, or nucleic acid sequencing (e.g., RNA sequencing or DNA sequencing). In some embodiments, DNA or RNA may be assayed by one or more of: isothermal DNA/RNA amplification methods (e.g., loop-mediated isothermal amplification (LAMP), helicase dependent amplification (HDA), rolling circle amplification (RCA), recombinase polymerase amplification (RPA)), immunoassays, electrochemical assays, surface-enhanced Raman spectroscopy (SERS), quantum dot (QD)-based assays, methylation detection, telomere length assays, molecular inversion probes, SEQENOM MassARRAY®, droplet digital PCR (ddPCR), CRISPR/Cas-based detection (e.g., CRISPR-typing PCR (ctPCR), specific high-sensitivity enzymatic reporter un-locking (SHERLOCK), DNA endonuclease targeted CRISPR trans reporter (DETECT®), and CRISPR-mediated analog multi-event recording apparatus (CAMERA)), and laser transmission spectroscopy (LTS).

The assay readouts may be quantified at one or more genomic loci (e.g., lung cancer-associated genomic loci) to generate the data indicative of lung cancer. For example, quantification of array hybridization or polymerase chain reaction (PCR) corresponding to a plurality of genomic loci (e.g., lung cancer-associated genomic loci) may generate data indicative of lung cancer. Assay readouts may comprise quantitative PCR (qPCR) values, digital PCR (dPCR) values, digital droplet PCR (ddPCR) values, fluorescence values, etc., or normalized values thereof. The assay may be a home use test configured to be performed in a home setting.

In some embodiments, multiple assays are used to process the biological samples of a subject. For example, a first assay may be used to process a first the biological sample obtained or derived from the subject to generate a first dataset; and based at least in part on the first dataset, a second assay different from said first assay may be used to process a second the biological sample obtained or derived from the subject to generate a second dataset indicative of said lung cancer. The first assay may be used to screen or process the biological samples of a set of subjects, while the second or subsequent assays may be used to screen or process the biological samples of a smaller subset of the set of subjects. The first assay may have a low cost and/or a high sensitivity of detecting one or more lung cancers (e.g., lung cancer), that is amenable to screening or processing the biological samples of a relatively large set of subjects. The second assay may have a higher cost and/or a higher specificity of detecting one or more lung cancers (e.g., lung cancer), that is amenable to screening or processing the biological samples of a relatively small set of subjects (e.g., a subset of the subjects screened using the first assay). The second assay may generate a second dataset having a specificity (e.g., for lung cancer) greater than the first dataset generated using the first assay. As an example, one or more the biological samples may be processed using a cfRNA assay on a large set of subjects and subsequently a metabolomics assay on a smaller subset of subjects, or vice versa. The smaller subset of subjects may be selected based at least in part on the results of the first assay.

Alternatively, multiple assays may be used to simultaneously process the biological samples of a subject. For example, a first assay may be used to process a first the biological sample obtained or derived from the subject to generate a first dataset indicative of lung cancer; and a second assay different from the first assay may be used to process a second the biological sample obtained or derived from the subject to generate a second dataset indicative of lung cancer. Any or all of the first dataset and the second dataset may then be analyzed to assess the lung cancer of the subject. For example, a single diagnostic index or diagnosis score can be generated based on a combination of the first dataset and the second dataset. As another example, separate diagnostic indexes or diagnosis scores can be generated based on the first dataset and the second dataset.

The biological samples may be processed using a metabolomics assay. For example, a metabolomics assay can be used to identify a quantitative measure (e.g., indicative of a presence, absence, or relative amount) of each of a plurality of lung cancer-associated metabolites in the biological sample of the subject. The metabolomics assay may be configured to process the biological samples such as a blood sample or a urine sample (or derivatives thereof) of the subject. A quantitative measure (e.g., indicative of a presence, absence, or relative amount) of lung cancer-associated metabolites in the biological sample may be indicative of lung cancer. The metabolites in the biological sample may be produced (e.g., as an end product or a byproduct) as a result of one or more metabolic pathways corresponding to lung cancer-associated genes. Assaying one or more metabolites of the biological sample may comprise isolating or extracting the metabolites from the biological sample. The metabolomics assay may be used to generate datasets indicative of the quantitative measure (e.g., indicative of a presence, absence, or relative amount) of each of a plurality of lung cancer-associated metabolites in the biological sample of the subject.

The biological samples may be processed using a proteomics assay. For example, a proteomics assay can be used to identify a quantitative measure (e.g., indicative of a presence, absence, or relative amount) of each of a plurality of lung cancer-associated proteins or polypeptides in the biological sample of the subject. The proteomics assay may be configured to process the biological samples such as a blood sample or a urine sample (or derivatives thereof) of the subject. A quantitative measure (e.g., indicative of a presence, absence, or relative amount) of lung cancer-associated proteins or polypeptides in the biological sample may be indicative of one or more lung cancers. The proteins or polypeptides in the biological sample may be produced (e.g., as an end product or a byproduct) as a result of one or more biochemical pathways corresponding to lung cancer-associated genes. Assaying one or more proteins or polypeptides of the biological sample may comprise isolating or extracting the proteins or polypeptides from the biological sample. The proteomics assay may be used to generate datasets indicative of the quantitative measure (e.g., indicative of a presence, absence, or relative amount) of each of a plurality of lung cancer-associated proteins or polypeptides in the biological sample of the subject.

### Trained Algorithms

After using one or more assays to process one or more of the biological samples derived from the subject to generate one or more datasets, a trained algorithm may be used to process one or more of the datasets (e.g., at each of a plurality of lung cancer-associated genomic loci) to determine a lung cancer-associated risk. For example, the trained algorithm may be used to determine qualitative or quantitative measures of sequences at each of the plurality of lung cancer-associated genomic loci or SNPs in the biological samples. The trained algorithm may be configured to identify the lung cancer with an accuracy of at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or more than 99% for at least about 25, at least about 50, at least about 100, at least about 150, at least about 200, at least about 250, at least about 300, at least about 350, at least about 400, at least about 450, at least about 500, or more than about 500 independent samples.

The present disclosure provides methods of processing or analyzing a bodily sample in a subject. The method may comprise analyzing the bodily sample to yield a first data set comprising at least one genetic polymorphism associated with a first pulmonary or respiratory disease or disorder. The method may further comprise analyzing a second data set comprising at least one genetic polymorphism associated with a second pulmonary or respiratory disease or disorder. The at least one genetic polymorphism of the first data set or the second data set may be associated with a risk of pulmonary or respiratory disease or disorder. The pulmonary or respiratory disease or disorder may be lung cancer. The pulmonary or respiratory disease or disorder may be airflow limitation. The pulmonary or respiratory disease or disorder may be COPD. Method as described herein may further comprise imaging a pulmonary nodule from the subject to determine at least one characteristic associated with the image of said pulmonary nodule. Methods as described herein may further comprise obtaining data with respect to at least one biomarker of the subject. Method as described herein may further comprise computer processing the first data set and the second data set to produce a third data set, and further analyzing the third data set and said at least one characteristic of the pulmonary nodule. Methods as described herein may further comprise determining the risk associated with the pulmonary nodule to an accuracy of at least about 80. The accuracy may be a percentage of a plurality of independent test samples corresponding to (1) a first plurality of subjects having a pulmonary nodule exhibiting malignancy to (2) a second plurality of subjects having a pulmonary nodule not exhibiting malignancy. Methods as described herein may further comprise electronically outputting a report that identifies the risk presented by the pulmonary nodule.

The trained algorithm may comprise a supervised machine learning algorithm. The trained algorithm may comprise a classification and regression tree (CART) algorithm. The supervised machine learning algorithm may comprise, for example, a Random Forest, a support vector machine (SVM), a neural network, or a deep learning algorithm. The trained algorithm may comprise an unsupervised machine learning algorithm.

The trained algorithm may be configured to accept a plurality of input variables and to produce one or more output values based on the plurality of input variables. The plurality of input variables may comprise one or more datasets indicative of a lung cancer. For example, an input variable may comprise a number of sequences corresponding to or aligning to each of the plurality of lung cancer-associated lung cancer-associated genomic loci or SNPs. In addition, the input variable may also include the presence of the at least clinical variables described herein in the subject. The plurality of input variables may also include clinical health data of a subject.

The trained algorithm may comprise a classifier, such that each of the one or more output values comprises one of a fixed number of possible values (e.g., a linear classifier, a logistic regression classifier, etc.) indicating a classification of the biological sample by the classifier. The trained algorithm may comprise a binary classifier, such that each of the one or more output values comprises one of two values (e.g., {0, 1}, {positive, negative}, or {high-risk, low-risk}) indicating a classification of the biological sample by the classifier. The trained algorithm may be another type of classifier, such that each of the one or more output values comprises one of more than two values (e.g., {0, 1, 2}, {positive, negative, or indeterminate}, or {high-risk, intermediate-risk, or low-risk}) indicating a classification of the biological sample by the classifier. The output values may comprise descriptive labels, numerical values, or a combination thereof. Some of the output values may comprise descriptive labels. Such descriptive labels may provide an identification or indication of the disease or disorder state of the subject, and may comprise, for example, positive, negative, high-risk, intermediate-risk, low-risk, or indeterminate. Such descriptive labels may provide an identification of a treatment for the subject's lung cancer, or a prioritization for early diagnosis through screening, and may comprise, for example, early diagnosis, a therapeutic intervention, a duration of the therapeutic intervention, and/or a dosage of the therapeutic intervention suitable to treat a lung cancer condition. Such descriptive labels may provide an identification of secondary clinical tests that may be appropriate to perform on the subject, and may comprise, for example, an imaging test, a blood test, a computed tomography (CT) scan, a magnetic resonance imaging (MRI) scan, an ultrasound scan, a chest X-ray, a positron emission tomography (PET) scan, a PET-CT scan, a biological cytology, an amniocentesis, a non-invasive prenatal test (NIPT), or any combination thereof. For example, such descriptive labels may provide a prognosis of the lung cancer of the subject. As another example, such descriptive labels may provide a relative assessment of the lung cancer (e.g., an estimated gestational age in number of days, weeks, or months) of the subject. Some descriptive labels may be mapped to numerical values, for example, by mapping “positive” to 1 and “negative” to 0).

Some of the output values may comprise numerical values, such as binary, integer, or continuous values. Such binary output values may comprise, for example, {0, 1}, {positive, negative}, or {high-risk, low-risk}. Such integer output values may comprise, for example, {0, 1, 2}. Such continuous output values may comprise, for example, a probability value of at least 0) and no more than 1. Such continuous output values may comprise, for example, an un-normalized probability value of at least 0. Such continuous output values may indicate a prognosis of the lung cancer of the subject. Some numerical values may be mapped to descriptive labels, for example, by mapping 1 to “positive” and ( ) to “negative.”

Some of the output values may be assigned based on one or more cutoff values. For example, a binary classification of samples may assign an output value of “positive” or 1 if the sample indicates that the subject has at least a 50% probability of having a lung cancer (e.g., lung cancer). For example, a binary classification of samples may assign an output value of “negative” or 0 if the sample indicates that the subject has less than a 50% probability of having a lung cancer (e.g., lung cancer). In this case, a single cutoff value of 50% is used to classify samples into one of the two possible binary output values. Examples of single cutoff values may include about 1%, about 2%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, and about 99%.

As another example, a classification of samples may assign an output value of “positive” or 1 if the sample indicates that the subject has a probability of having a lung cancer of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more. The classification of samples may assign an output value of “positive” or 1 if the sample indicates that the subject has a probability of having a lung cancer of more than about 50%, more than about 55%, more than about 60%, more than about 65%, more than about 70%, more than about 75%, more than about 80%, more than about 85%, more than about 90%, more than about 91%, more than about 92%, more than about 93%, more than about 94%, more than about 95%, more than about 96%, more than about 97%, more than about 98%, or more than about 99%.

The classification of samples may assign an output value of “negative” or 0 if the sample indicates that the subject has a probability of having a lung cancer of less than about 50%, less than about 45%, less than about 40%, less than about 35%, less than about 30%, less than about 25%, less than about 20%, less than about 15%, less than about 10%, less than about 9%, less than about 8%, less than about 7%, less than about 6%, less than about 5%, less than about 4%, less than about 3%, less than about 2%, or less than about 1%. The classification of samples may assign an output value of “negative” or 0 if the sample indicates that the subject has a probability of having a lung cancer of no more than about 50%, no more than about 45%, no more than about 40%, no more than about 35%, no more than about 30%, no more than about 25%, no more than about 20%, no more than about 15%, no more than about 10%, no more than about 9%, no more than about 8%, no more than about 7%, no more than about 6%, no more than about 5%, no more than about 4%, no more than about 3%, no more than about 2%, or no more than about 1%.

The classification of samples may assign an output value of “indeterminate” or 2 if the sample is not classified as “positive”, “negative”, 1, or 0. In this case, a set of two cutoff values is used to classify samples into one of the three possible output values. Examples of sets of cutoff values may include {1%, 99%}, {2%, 98%}, {5%, 95%}, {10%, 90%}, {15%, 85%}, {20%, 80%}, {25%, 75%}, {30%, 70%}, {35%, 65%}, {40%, 60%}, and {45%, 55%}. Similarly, sets of n cutoff values may be used to classify samples into one of n+1 possible output values, where n is any positive integer.

The trained algorithm may be trained with a plurality of independent training samples. Each of the independent training samples may comprise a biological sample from a subject, associated datasets obtained by assaying the biological sample (as described elsewhere herein), and one or more known output values corresponding to the biological sample (e.g., a clinical diagnosis, prognosis, absence, or treatment efficacy of a lung cancer of the subject). Independent training samples may comprise the biological samples and associated datasets and outputs obtained or derived from a plurality of different subjects. Independent training samples may comprise the biological samples and associated datasets and outputs obtained at a plurality of different time points from the same subject (e.g., on a regular basis such as weekly, biweekly, or monthly). Independent training samples may be associated with presence of the lung cancer (e.g., training samples comprising the biological samples and associated datasets and outputs obtained or derived from a plurality of subjects known to have the lung cancer). Independent training samples may be associated with absence of the lung cancer (e.g., training samples comprising the biological samples and associated datasets and outputs obtained or derived from a plurality of subjects who are known to not have a previous diagnosis of the lung cancer or who have received a negative test result for the lung cancer).

The trained algorithm may be trained with at least about 5, at least about 10, at least about 15, at least about 20, at least about 25, at least about 30, at least about 35, at least about 40, at least about 45, at least about 50, at least about 100, at least about 150, at least about 200, at least about 250, at least about 300, at least about 350, at least about 400, at least about 450, or at least about 500 independent training samples. The independent training samples may comprise the biological samples associated with presence of the lung cancer and/or the biological samples associated with absence of the lung cancer. The trained algorithm may be trained with no more than about 500, no more than about 450, no more than about 400, no more than about 350, no more than about 300, no more than about 250, no more than about 200, no more than about 150, no more than about 100, or no more than about 50 independent training samples associated with presence of the lung cancer. In some embodiments, the biological sample is independent of samples used to train the trained algorithm.

The trained algorithm may be trained with a first number of independent training samples associated with presence of the lung cancer and a second number of independent training samples associated with absence of the lung cancer. The first number of independent training samples associated with presence of the lung cancer may be no more than the second number of independent training samples associated with absence of the lung cancer. The first number of independent training samples associated with presence of the lung cancer may be equal to the second number of independent training samples associated with absence of the lung cancer. The first number of independent training samples associated with presence of the lung cancer may be greater than the second number of independent training samples associated with absence of the lung cancer.

The trained algorithm may be configured to identify the lung cancer at an accuracy of at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more; for at least about 5, at least about 10, at least about 15, at least about 20, at least about 25, at least about 30, at least about 35, at least about 40, at least about 45, at least about 50, at least about 100, at least about 150, at least about 200, at least about 250, at least about 300, at least about 350, at least about 400, at least about 450, or at least about 500 independent training samples. The accuracy of identifying the lung cancer by the trained algorithm may be calculated as the percentage of independent test samples (e.g., subjects known to have the lung cancer or subjects with negative clinical test results for the lung cancer) that are correctly identified or classified as having or not having the lung cancer.

The trained algorithm may be configured to identify the risk associated with a pulmonary nodule at an accuracy of at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more; for at least about 5, at least about 10, at least about 15, at least about 20, at least about 25, at least about 30, at least about 35, at least about 40, at least about 45, at least about 50, at least about 100, at least about 150, at least about 200, at least about 250, at least about 300, at least about 350, at least about 400, at least about 450, or at least about 500 independent training samples. The accuracy of identifying the lung cancer by the trained algorithm may be calculated as the percentage of independent test samples (e.g., subjects known to have the lung cancer or subjects with negative clinical test results for the lung cancer) that are correctly identified or classified as having or not having the lung cancer.

The trained algorithm may be configured to identify the lung cancer with a positive predictive value (PPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The PPV of identifying the lung cancer using the trained algorithm may be calculated as the percentage of the biological samples identified or classified as having the lung cancer that correspond to subjects that truly have the lung cancer.

The trained algorithm may be configured to identify the risk associated with a pulmonary nodule with a positive predictive value (PPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The PPV of identifying the lung cancer using the trained algorithm may be calculated as the percentage of the biological samples identified or classified as having the lung cancer that correspond to subjects that truly have the lung cancer.

The trained algorithm may be configured to identify the lung cancer with a negative predictive value (NPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The NPV of identifying the lung cancer using the trained algorithm may be calculated as the percentage of the biological samples identified or classified as not having the lung cancer that correspond to subjects that truly do not have the lung cancer.

The trained algorithm may be configured to identify the risk associated with a pulmonary nodule with a negative predictive value (NPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The NPV of identifying the lung cancer using the trained algorithm may be calculated as the percentage of the biological samples identified or classified as not having the lung cancer that correspond to subjects that truly do not have the lung cancer.

The trained algorithm may be configured to identify the lung cancer with a clinical sensitivity at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about 99.999%, or more. The clinical sensitivity of identifying the lung cancer using the trained algorithm may be calculated as the percentage of independent test samples associated with presence of the lung cancer (e.g., subjects known to have the lung cancer) that are correctly identified or classified as having the lung cancer.

The trained algorithm may be configured to identify the risk presented by a pulmonary nodule with a clinical sensitivity at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about 99.999%, or more. The clinical sensitivity of identifying the lung cancer using the trained algorithm may be calculated as the percentage of independent test samples associated with presence of the lung cancer (e.g., subjects known to have the lung cancer) that are correctly identified or classified as having the lung cancer.

The trained algorithm may be configured to identify the lung cancer with a clinical specificity of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about 99.999%, or more. The clinical specificity of identifying the lung cancer using the trained algorithm may be calculated as the percentage of independent test samples associated with absence of the lung cancer (e.g., subjects with negative clinical test results for the lung cancer) that are correctly identified or classified as not having the lung cancer.

The trained algorithm may be configured to identify the risk associated with a pulmonary nodule with a clinical specificity of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about 99.999%, or more. The clinical specificity of identifying the lung cancer using the trained algorithm may be calculated as the percentage of independent test samples associated with absence of the lung cancer (e.g., subjects with negative clinical test results for the lung cancer) that are correctly identified or classified as not having the lung cancer.

The trained algorithm may be configured to identify the lung cancer with an Area-Under-Curve (AUC) of at least about 0.50, at least about 0.55, at least about 0.60, at least about 0.65, at least about 0.70, at least about 0.75, at least about 0.80, at least about 0.81, at least about 0.82, at least about 0.83, at least about 0.84, at least about 0.85, at least about 0.86, at least about 0.87, at least about 0.88, at least about 0.89, at least about 0.90, at least about 0.91, at least about 0.92, at least about 0.93, at least about 0.94, at least about 0.95, at least about 0.96, at least about 0.97, at least about 0.98, at least about 0.99, or more. The AUC may be calculated as an integral of the Receiver Operator Characteristic (ROC) curve (e.g., the area under the ROC curve) associated with the trained algorithm in classifying the biological samples as having or not having the lung cancer.

The trained algorithm may be configured to identify the risk associated with a pulmonary nodule with an Area-Under-Curve (AUC) of at least about 0.50, at least about 0.55, at least about 0.60, at least about 0.65, at least about 0.70, at least about 0.75, at least about 0.80, at least about 0.81, at least about 0.82, at least about 0.83, at least about 0.84, at least about 0.85, at least about 0.86, at least about 0.87, at least about 0.88, at least about 0.89, at least about 0.90, at least about 0.91, at least about 0.92, at least about 0.93, at least about 0.94, at least about 0.95, at least about 0.96, at least about 0.97, at least about 0.98, at least about 0.99, or more. The AUC may be calculated as an integral of the Receiver Operator Characteristic (ROC) curve (e.g., the area under the ROC curve) associated with the trained algorithm in classifying the biological samples as having or not having the lung cancer.

The trained algorithm may be adjusted or tuned to improve one or more of the performance, accuracy, PPV, NPV, clinical sensitivity, clinical specificity, or AUC of identifying the lung cancer. The trained algorithm may be adjusted or tuned by adjusting parameters of the trained algorithm (e.g., a set of cutoff values used to classify the biological sample as described elsewhere herein, or weights of a neural network). The trained algorithm may be adjusted or tuned continuously during the training process or after the training process has completed.

After the trained algorithm is initially trained, a subset of the inputs may be identified as most influential or most important to be included for making high-quality classifications. For example, a subset of the plurality of lung cancer-associated genomic loci may be identified as most influential or most important to be included for making high-quality classifications or identifications of lung cancers (or sub-types of lung cancers). The plurality of lung cancer-associated genomic loci or a subset thereof may be ranked based on classification metrics indicative of each genomic locus's influence or importance toward making high-quality classifications or identifications of lung cancers (or sub-types of lung cancers). Such metrics may be used to reduce, in some cases significantly, the number of input variables (e.g., predictor variables) that may be used to train the trained algorithm to a desired performance level (e.g., based on a desired minimum accuracy, PPV, NPV, clinical sensitivity, clinical specificity, AUC, or a combination thereof). For example, if training the trained algorithm with a plurality comprising several dozen or hundreds of input variables in the trained algorithm results in an accuracy of classification of more than 99%, then training the trained algorithm instead with only a selected subset of no more than about 5, no more than about 10, no more than about 15, no more than about 20, no more than about 25, no more than about 30, no more than about 35, no more than about 40, no more than about 45, no more than about 50, or no more than about 100 such most influential or most important input variables among the plurality can yield decreased but still acceptable accuracy of classification (e.g., at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99%). The subset may be selected by rank-ordering the entire plurality of input variables and selecting a predetermined number (e.g., no more than about 5, no more than about 10, no more than about 15, no more than about 20, no more than about 25, no more than about 30, no more than about 35, no more than about 40, no more than about 45, no more than about 50, or no more than about 100) of input variables with the best classification metrics.

### Identifying or Monitoring a Lung Cancer

After using a trained algorithm to process the dataset, the lung cancer may be identified or monitored in the subject. The identification may be based at least in part on quantitative measures of sequence reads of the dataset at a panel of lung cancer-associated genomic loci or SNPs (e.g., quantitative measures of RNA transcripts or DNA at the lung cancer-associated genomic loci), methylation detection, telomere length assays, proteomic data comprising quantitative measures of proteins of the dataset at a panel of lung cancer-associated proteins, and/or metabolome data comprising quantitative measures of a panel of lung cancer-associated metabolites.

The lung cancer may be identified in the subject at an accuracy of at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The accuracy of identifying the lung cancer by the trained algorithm may be calculated as the percentage of independent test samples (e.g., subjects known to have the lung cancer or subjects with negative clinical test results for the lung cancer) that are correctly identified or classified as having or not having the lung cancer.

The lung cancer may be identified in the subject with a positive predictive value (PPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The PPV of identifying the lung cancer using the trained algorithm may be calculated as the percentage of the biological samples identified or classified as having the lung cancer that correspond to subjects that truly have the lung cancer.

The lung cancer may be identified in the subject with a negative predictive value (NPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The NPV of identifying the lung cancer using the trained algorithm may be calculated as the percentage of the biological samples identified or classified as not having the lung cancer that correspond to subjects that truly do not have the lung cancer.

The lung cancer may be identified in the subject with a clinical sensitivity of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about 99.999%, or more. The clinical sensitivity of identifying the lung cancer using the trained algorithm may be calculated as the percentage of independent test samples associated with presence of the lung cancer (e.g., subjects known to have the lung cancer) that are correctly identified or classified as having the lung cancer.

The lung cancer may be identified in the subject with a clinical specificity of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about 99.999%, or more. The clinical specificity of identifying the lung cancer using the trained algorithm may be calculated as the percentage of independent test samples associated with absence of the lung cancer (e.g., subjects with negative clinical test results for the lung cancer) that are correctly identified or classified as not having the lung cancer.

In an aspect, the present disclosure provides a method for determining that a subject is at risk of developing lung cancer, comprising assaying a biological sample derived from the subject to generate a dataset that is indicative of said lung cancer-associated risk at a specificity of at least 80%, and using a trained algorithm that is trained on samples independent of the biological sample to determine that the subject is at risk of developing lung cancer at an accuracy of at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more.

After the lung cancer is identified in a subject, a sub-type of the lung cancer (e.g., selected from among a plurality of sub-types of the lung cancer) may further be identified. The sub-type of the lung cancer may be determined based at least in part on the quantitative measures of sequence reads of the dataset at a panel of lung cancer-associated genomic loci (e.g., quantitative measures of RNA transcripts or DNA at the lung cancer-associated genomic loci), methylation detection, telomere length assays, proteomic data comprising quantitative measures of proteins of the dataset at a panel of lung cancer-associated proteins, and/or metabolome data comprising quantitative measures of a panel of lung cancer-associated metabolites. For example, the subject may be identified as being at risk of a sub-type of lung. After identifying the subject as being at risk of developing a sub-type of lung cancer, a clinical intervention for the subject may be selected based at least in part on the sub-type of lung cancer for which the subject is identified as being at risk. In some embodiments, the clinical intervention is selected from a plurality of clinical interventions including early diagnosis or screening.

In some embodiments, the trained algorithm may determine that the subject is at risk of developing lung cancer of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more.

The trained algorithm may determine that the subject is at risk of developing lung cancer at an accuracy of at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about 99.999%, or more.

Upon identifying the subject as having the lung cancer, the subject may be optionally provided with a therapeutic intervention (e.g., prescribing an appropriate course of treatment to treat the lung cancer of the subject). The therapeutic intervention may comprise a prescription of an effective dose of a drug, a further testing or evaluation of the lung cancer, a further monitoring of the lung cancer, an induction or inhibition of labor, or a combination thereof. If the subject is currently being treated for the lung cancer with a course of treatment, the therapeutic intervention may comprise a subsequent different course of treatment (e.g., to increase treatment efficacy due to non-efficacy of the current course of treatment).

The therapeutic intervention, screening, or early diagnosis may comprise recommending the subject for a secondary clinical test to confirm a diagnosis of the lung cancer. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, a biological cytology, an amniocentesis, a non-invasive prenatal test (NIPT), or any combination thereof.

The quantitative measures of sequence reads of the dataset at the panel of lung cancer-associated genomic loci (e.g., quantitative measures of RNA transcripts or DNA at the lung cancer-associated genomic loci), methylation detection, telomere length assays, proteomic data comprising quantitative measures of proteins of the dataset at a panel of lung cancer-associated proteins, and/or metabolome data comprising quantitative measures of a panel of lung cancer-associated metabolites may be assessed over a duration of time to monitor a patient (e.g., subject who has lung cancer or who is being treated for lung cancer). In such cases, the quantitative measures of the dataset of the patient may change during the course of treatment. For example, the quantitative measures of the dataset of a patient with decreasing risk of the lung cancer due to an effective treatment may shift toward the profile or distribution of a healthy subject (e.g., a subject without a lung cancer complication). Conversely, for example, the quantitative measures of the dataset of a patient with increasing risk of the lung cancer due to an ineffective treatment may shift toward the profile or distribution of a subject with higher risk of the lung cancer or a more advanced lung cancer.

The lung cancer of the subject may be monitored by monitoring a course of treatment for treating the lung cancer of the subject. The monitoring may comprise assessing the lung cancer of the subject at two or more time points. The assessing may be based at least on the quantitative measures of sequence reads of the dataset at a panel of lung cancer-associated genomic loci (e.g., quantitative measures of RNA transcripts or DNA at the lung cancer-associated genomic loci), proteomic data comprising quantitative measures of proteins of the dataset at a panel of lung cancer-associated proteins, and/or metabolome data comprising quantitative measures of a panel of lung cancer-associated metabolites determined at each of the two or more time points.

In some embodiments, a difference in the quantitative measures of sequence reads of the dataset at a panel of lung cancer-associated genomic loci (e.g., quantitative measures of RNA transcripts or DNA at the lung cancer-associated genomic loci), proteomic data comprising quantitative measures of proteins of the dataset at a panel of lung cancer-associated proteins, and/or metabolome data comprising quantitative measures of a panel of lung cancer-associated metabolites determined between the two or more time points may be indicative of one or more clinical indications, such as (i) a diagnosis of the lung cancer of the subject. (ii) a prognosis of the lung cancer of the subject, (iii) an increased risk of the lung cancer of the subject. (iv) a decreased risk of the lung cancer of the subject. (v) an efficacy of the course of treatment for treating the lung cancer of the subject, and (vi) a non-efficacy of the course of treatment for treating the lung cancer of the subject.

In some embodiments, a difference in the quantitative measures of sequence reads of the dataset at a panel of lung cancer-associated genomic loci (e.g., quantitative measures of RNA transcripts or DNA at the lung cancer-associated genomic loci), proteomic data comprising quantitative measures of proteins of the dataset at a panel of lung cancer-associated proteins, and/or metabolome data comprising quantitative measures of a panel of lung cancer-associated metabolites determined between the two or more time points may be indicative of a diagnosis of the lung cancer of the subject. For example, if the lung cancer was not detected in the subject at an earlier time point but was detected in the subject at a later time point, then the difference is indicative of a diagnosis of the lung cancer of the subject. A clinical action or decision may be made based on this indication of diagnosis of the lung cancer of the subject, such as, for example, prescribing a new therapeutic intervention for the subject. The clinical action or decision may comprise screening. The clinical action or decision may comprise recommending the subject for a secondary clinical test to confirm the diagnosis of the lung cancer. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, a biological cytology, an amniocentesis, a non-invasive prenatal test (NIPT), or any combination thereof.

In some embodiments, a difference in the quantitative measures of sequence reads of the dataset at a panel of lung cancer-associated genomic loci (e.g., quantitative measures of RNA transcripts or DNA at the lung cancer-associated genomic loci), proteomic data comprising quantitative measures of proteins of the dataset at a panel of lung cancer-associated proteins, and/or metabolome data comprising quantitative measures of a panel of lung cancer-associated metabolites determined between the two or more time points may be indicative of a prognosis of the lung cancer of the subject.

In some embodiments, a difference in the quantitative measures of sequence reads of the dataset at a panel of lung cancer-associated genomic loci (e.g., quantitative measures of RNA transcripts or DNA at the lung cancer-associated genomic loci), proteomic data comprising quantitative measures of proteins of the dataset at a panel of lung cancer-associated proteins, and/or metabolome data comprising quantitative measures of a panel of lung cancer-associated metabolites determined between the two or more time points may be indicative of the subject having an increased risk of the lung cancer. For example, if the lung cancer was detected in the subject both at an earlier time point and at a later time point, and if the difference is a negative difference (e.g., the quantitative measures of sequence reads of the dataset at a panel of lung cancer-associated genomic loci (e.g., quantitative measures of RNA transcripts or DNA at the lung cancer-associated genomic loci), methylation detection, telomere length assays, proteomic data comprising quantitative measures of proteins of the dataset at a panel of lung cancer-associated proteins, and/or metabolome data comprising quantitative measures of a panel of lung cancer-associated metabolites increased from the earlier time point to the later time point), then the difference may be indicative of the subject having an increased risk of the lung cancer. A clinical action or decision may be made based on this indication of the increased risk of the lung cancer, e.g., prescribing a new therapeutic intervention or switching therapeutic interventions (e.g., ending a current treatment and prescribing a new treatment) for the subject. The clinical action or decision may comprise screening. The clinical action or decision may comprise recommending the subject for a secondary clinical test to confirm the increased risk of the lung cancer. The clinical action or decision may comprise screening. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, a biological cytology, an amniocentesis, a non-invasive prenatal test (NIPT), or any combination thereof.

In some embodiments, a difference in the quantitative measures of sequence reads of the dataset at a panel of lung cancer-associated genomic loci (e.g., quantitative measures of RNA transcripts or DNA at the lung cancer-associated genomic loci), proteomic data comprising quantitative measures of proteins of the dataset at a panel of lung cancer-associated proteins, and/or metabolome data comprising quantitative measures of a panel of lung cancer-associated metabolites determined between the two or more time points may be indicative of the subject having a decreased risk of the lung cancer. For example, if the lung cancer was detected in the subject both at an earlier time point and at a later time point, and if the difference is a positive difference (e.g., the quantitative measures of sequence reads of the dataset at a panel of lung cancer-associated genomic loci (e.g., quantitative measures of RNA transcripts or DNA at the lung cancer-associated genomic loci), proteomic data comprising quantitative measures of proteins of the dataset at a panel of lung cancer-associated proteins, and/or metabolome data comprising quantitative measures of a panel of lung cancer-associated metabolites decreased from the earlier time point to the later time point), then the difference may be indicative of the subject having a decreased risk of the lung cancer. A clinical action or decision may be made based on this indication of the decreased risk of the lung cancer (e.g., continuing or ending a current therapeutic intervention or screening intervention) for the subject. The clinical action or decision may comprise recommending the subject for a secondary clinical test to confirm the decreased risk of the lung cancer. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, a biological cytology, an amniocentesis, a non-invasive prenatal test (NIPT), or any combination thereof.

In some embodiments, a difference in the quantitative measures of sequence reads of the dataset at a panel of lung cancer-associated genomic loci (e.g., quantitative measures of RNA transcripts or DNA at the lung cancer-associated genomic loci), proteomic data comprising quantitative measures of proteins of the dataset at a panel of lung cancer-associated proteins, and/or metabolome data comprising quantitative measures of a panel of lung cancer-associated metabolites determined between the two or more time points may be indicative of an efficacy of the course of treatment for treating the lung cancer of the subject. For example, if the lung cancer was detected in the subject at an earlier time point but was not detected in the subject at a later time point, then the difference may be indicative of an efficacy of the course of treatment for treating the lung cancer of the subject. A clinical action or decision may be made based on this indication of the efficacy of the course of treatment for treating the lung cancer of the subject, e.g., continuing or ending a current therapeutic intervention for the subject. The clinical action or decision may comprise recommending the subject for a secondary clinical test to confirm the efficacy of the course of treatment for treating the lung cancer. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, a biological cytology, an amniocentesis, a non-invasive prenatal test (NIPT), or any combination thereof.

In some embodiments, a difference in the quantitative measures of sequence reads of the dataset at a panel of lung cancer-associated genomic loci (e.g., quantitative measures of RNA transcripts or DNA at the lung cancer-associated genomic loci), proteomic data comprising quantitative measures of proteins of the dataset at a panel of lung cancer-associated proteins, and/or metabolome data comprising quantitative measures of a panel of lung cancer-associated metabolites determined between the two or more time points may be indicative of a non-efficacy of the course of treatment for treating the lung cancer of the subject. For example, if the lung cancer was detected in the subject both at an earlier time point and at a later time point, and if the difference is a negative or zero difference (e.g., the quantitative measures of sequence reads of the dataset at a panel of lung cancer-associated genomic loci (e.g., quantitative measures of RNA transcripts or DNA at the lung cancer-associated genomic loci), proteomic data comprising quantitative measures of proteins of the dataset at a panel of lung cancer-associated proteins, and/or metabolome data comprising quantitative measures of a panel of lung cancer-associated metabolites increased or remained at a constant level from the earlier time point to the later time point), and if an efficacious treatment was indicated at an earlier time point, then the difference may be indicative of a non-efficacy of the course of treatment for treating the lung cancer of the subject. A clinical action or decision may be made based on this indication of the non-efficacy of the course of treatment for treating the lung cancer of the subject, e.g., ending a current therapeutic intervention and/or switching to (e.g., prescribing) a different new therapeutic intervention for the subject. The clinical action or decision may comprise recommending the subject for a secondary clinical test to confirm the non-efficacy of the course of treatment for treating the lung cancer. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, a biological cytology, an amniocentesis, a non-invasive prenatal test (NIPT), or any combination thereof.

In another aspect, the present disclosure provides a computer-implemented method for predicting a risk of developing lung cancer of a subject, comprising: (a) receiving clinical health data of the subject, wherein the clinical health data comprises a plurality of quantitative or categorical measures of said subject; (b) using a trained algorithm to process the clinical health data of the subject to determine a risk score indicative of the risk of developing lung cancer of the subject; and (c) electronically outputting a report indicative of the risk score indicative of the risk developing lung cancer of the subject.

In some embodiments, for example, the clinical health data comprises one or more quantitative measures of the subject, such as age, weight, height, body mass index (BMI), blood pressure, heart rate, glucose levels, number of previous pregnancies, and number of previous cancer, including lung cancer, incidences. As another example, the clinical health data can comprise one or more categorical measures, such as race, ethnicity, history of medication or other clinical treatment, history of tobacco use, history of alcohol consumption, daily activity or fitness level, genetic test results, blood test results, imaging results, and fetal screening results.

In some embodiments, the computer-implemented method for predicting a risk of developing lung cancer of a subject is performed using a computer or mobile device application. For example, a subject can use a computer or mobile device application to input her own clinical health data, including quantitative and/or categorical measures. The computer or mobile device application can then use a trained algorithm to process the clinical health data to determine a risk score indicative of the risk of developing lung cancer of the subject. The computer or mobile device application can then display a report indicative of the risk score indicative of the risk of developing lung cancer of the subject.

In some embodiments, the risk score indicative of the risk of developing lung cancer of the subject can be refined by performing one or more subsequent clinical tests for the subject. For example, the subject can be referred by a physician for one or more subsequent clinical tests (e.g., an ultrasound imaging or a blood test) based on the initial risk score. Next, the computer or mobile device application may process results from the one or more subsequent clinical tests using a trained algorithm to determine an updated risk score indicative of the risk of developing lung cancer of the subject.

In some embodiments, the risk score comprises a likelihood of the subject having developing lung cancer within a pre-determined duration of time. For example, the pre-determined duration of time may be about 1 hour, about 2 hours, about 4 hours, about 6 hours, about 8 hours, about 10 hours, about 12 hours, about 14 hours, about 16 hours, about 18 hours, about 20 hours, about 22 hours, about 24 hours, about 1.5 days, about 2 days, about 2.5 days, about 3 days, about 3.5 days, about 4 days, about 4.5 days, about 5 days, about 5.5 days, about 6 days, about 6.5 days, about 7 days, about 8 days, about 9 days, about 10 days, about 12 days, about 14 days, about 3 weeks, about 4 weeks, about 5 weeks, about 6 weeks, about 7 weeks, about 8 weeks, about 9 weeks, about 10 weeks, about 11 weeks, about 12 weeks, about 13 weeks, or more than about 13 weeks.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

### Computing System

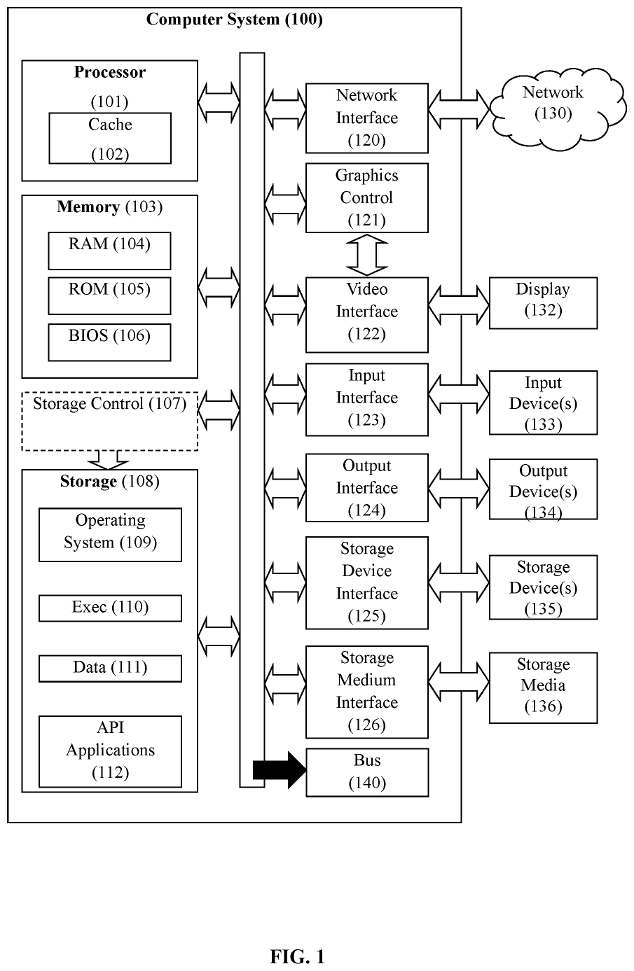

Referring to FIG. 1, a block diagram is shown depicting an exemplary machine that includes a computer system 100 (e.g., a processing or computing system) within which a set of instructions can execute for causing a device to perform or execute any one or more of the aspects and/or methodologies for static code scheduling of the present disclosure. The components in FIG. 1 are examples only and do not limit the scope of use or functionality of any hardware, software, embedded logic component, or a combination of two or more such components implementing particular embodiments.

Computer system 100 may include one or more processors 101, a memory 103, and a storage 108 that communicate with each other, and with other components, via a bus 140. The bus 140 may also link a display 132, one or more input devices 133 (which may, for example, include a keypad, a keyboard, a mouse, a stylus, etc.), one or more output devices 134, one or more storage devices 135, and various tangible storage media 136. All of these elements may interface directly or via one or more interfaces or adaptors to the bus 140. For instance, the various tangible storage media 136 can interface with the bus 140 via storage medium interface 126. Computer system 100 may have any suitable physical form, including but not limited to one or more integrated circuits (ICs), printed circuit boards (PCBs), mobile handheld devices (such as mobile telephones or PDAs), laptop or notebook computers, distributed computer systems, computing grids, or servers.

Computer system 100 includes one or more processor(s) 101 (e.g., central processing units (CPUs) or general-purpose graphics processing units (GPGPUs)) that carry out functions. Processor(s) 101 optionally contains a cache memory unit 102 for temporary local storage of instructions, data, or computer addresses. Processor(s) 101 are configured to assist in execution of computer readable instructions. Computer system 100 may provide functionality for the components depicted in FIG. 1 as a result of the processor(s) 101 executing non-transitory, processor-executable instructions embodied in one or more tangible computer-readable storage media, such as memory 103, storage 108, storage devices 135, and/or storage medium 136. The computer-readable media may store software that implements particular embodiments, and processor(s) 101 may execute the software. Memory 103 may read the software from one or more other computer-readable media (such as mass storage device(s) 135, 136) or from one or more other sources through a suitable interface, such as network interface 120. The software may cause processor(s) 101 to carry out one or more processes or one or more steps of one or more processes described or illustrated herein. Carrying out such processes or steps may include defining data structures stored in memory 103 and modifying the data structures as directed by the software.

The memory 103 may include various components (e.g., machine readable media) including, but not limited to, a random access memory component (e.g., RAM 104) (e.g., static RAM (SRAM), dynamic RAM (DRAM), ferroelectric random access memory (FRAM), phase-change random access memory (PRAM), etc.), a read-only memory component (e.g., ROM 105), and any combinations thereof. ROM 105 may act to communicate data and instructions unidirectionally to processor(s) 101, and RAM 104 may act to communicate data and instructions bidirectionally with processor(s) 101. ROM 105 and RAM 104 may include any suitable tangible computer-readable media described below. In one example, a basic input/output system 106 (BIOS), including basic routines that help to transfer information between elements within computer system 100, such as during start-up, may be stored in the memory 103.

Fixed storage 108 is connected bidirectionally to processor(s) 101, optionally through storage control unit 107. Fixed storage 108 provides additional data storage capacity and may also include any suitable tangible computer-readable media described herein. Storage 108 may be used to store operating system 109, executable(s) 110, data 111, applications 112 (application programs), and the like. Storage 108 can also include an optical disk drive, a solid-state memory device (e.g., flash-based systems), or a combination of any of the above. Information in storage 108 may, in appropriate cases, be incorporated as virtual memory in memory 103.

In one example, storage device(s) 135 may be removably interfaced with computer system 100 (e.g., via an external port connector (not shown)) via a storage device interface 125. Particularly, storage device(s) 135 and an associated machine-readable medium may provide non-volatile and/or volatile storage of machine-readable instructions, data structures, program modules, and/or other data for the computer system 100. In one example, software may reside, completely or partially, within a machine-readable medium on storage device(s) 135. In another example, software may reside, completely or partially, within processor(s) 101.

Bus 140 connects a wide variety of subsystems. Herein, reference to a bus may encompass one or more digital signal lines serving a common function, where appropriate. Bus 140 may be any of several types of bus structures including, but not limited to, a memory bus, a memory controller, a peripheral bus, a local bus, and any combinations thereof, using any of a variety of bus architectures. As an example and not by way of limitation, such architectures include an Industry Standard Architecture (ISA) bus, an Enhanced ISA (EISA) bus, a Micro Channel Architecture (MCA) bus, a Video Electronics Standards Association local bus (VLB), a Peripheral Component Interconnect (PCI) bus, a PCI-Express (PCI-X) bus, an Accelerated Graphics Port (AGP) bus, HyperTransport (HTX) bus, serial advanced technology attachment (SATA) bus, and any combinations thereof.

Computer system 100 may also include an input device 133. In one example, a user of computer system 100 may enter commands and/or other information into computer system 100 via input device(s) 133. Examples of an input device(s) 133 include, but are not limited to, an alpha-numeric input device (e.g., a keyboard), a pointing device (e.g., a mouse or touchpad), a touchpad, a touch screen, a multi-touch screen, a joystick, a stylus, a gamepad, an audio input device (e.g., a microphone, a voice response system, etc.), an optical scanner, a video or still image capture device (e.g., a camera), and any combinations thereof. In some embodiments, the input device is a Kinect, Leap Motion, or the like. Input device(s) 133 may be interfaced to bus 140 via any of a variety of input interfaces 123 (e.g., input interface 123) including, but not limited to, serial, parallel, game port, USB, FIREWIRE, THUNDERBOLT, or any combination of the above.

In particular embodiments, when computer system 100 is connected to network 130, computer system 100 may communicate with other devices, specifically mobile devices and enterprise systems, distributed computing systems, cloud storage systems, cloud computing systems, and the like, connected to network 130. Communications to and from computer system 100 may be sent through network interface 120. For example, network interface 120 may receive incoming communications (such as requests or responses from other devices) in the form of one or more packets (such as Internet Protocol (IP) packets) from network 130, and computer system 100 may store the incoming communications in memory 103 for processing. Computer system 100 may similarly store outgoing communications (such as requests or responses to other devices) in the form of one or more packets in memory 103 and communicated to network 130 from network interface 120. Processor(s) 101 may access these communication packets stored in memory 103 for processing.

Examples of the network interface 120 include, but are not limited to, a network interface card, a modem, and any combination thereof. Examples of a network 130 or network segment 130 include, but are not limited to, a distributed computing system, a cloud computing system, a wide area network (WAN) (e.g., the Internet, an enterprise network), a local area network (LAN) (e.g., a network associated with an office, a building, a campus or other relatively small geographic space), a telephone network, a direct connection between two computing devices, a peer-to-peer network, and any combinations thereof. A network, such as network 130, may employ a wired and/or a wireless mode of communication. In general, any network topology may be used.

Information and data can be displayed through a display 132. Examples of a display 132 include, but are not limited to, a cathode ray tube (CRT), a liquid crystal display (LCD), a thin film transistor liquid crystal display (TFT-LCD), an organic liquid crystal display (OLED) such as a passive-matrix OLED (PMOLED) or active-matrix OLED (AMOLED) display, a plasma display, and any combinations thereof. The display 132 can interface to the processor(s) 101, memory 103, and fixed storage 108, as well as other devices, such as input device(s) 133, via the bus 140. The display 132 is linked to the bus 140 via a video interface 122, and transport of data between the display 132 and the bus 140 can be controlled via the graphics control 121. In some embodiments, the display is a video projector. In some embodiments, the display is a head-mounted display (HMD) such as a VR headset. In further embodiments, suitable VR headsets include, by way of non-limiting examples, HTC Vive, Oculus Rift, Samsung Gear VR, Microsoft HoloLens, Razer OSVR, FOVE VR, Zeiss VR One, Avegant Glyph, Freefly VR headset, and the like. In still further embodiments, the display is a combination of devices such as those disclosed herein.

In addition to a display 132, computer system 100 may include one or more other peripheral output devices 134 including, but not limited to, an audio speaker, a printer, a storage device, and any combinations thereof. Such peripheral output devices may be connected to the bus 140 via an output interface 124. Examples of an output interface 124 include, but are not limited to, a serial port, a parallel connection, a USB port, a FIREWIRE port, a THUNDERBOLT port, and any combinations thereof.

In addition, or as an alternative, computer system 100 may provide functionality as a result of logic hardwired or otherwise embodied in a circuit, which may operate in place of or together with software to execute one or more processes or one or more steps of one or more processes described or illustrated herein. Reference to software in this disclosure may encompass logic, and reference to logic may encompass software. Moreover, reference to a computer-readable medium may encompass a circuit (such as an IC) storing software for execution, a circuit embodying logic for execution, or both, where appropriate. The present disclosure encompasses any suitable combination of hardware, software, or both.

Those of skill in the art will appreciate that the various illustrative logical blocks, modules, circuits, and algorithm steps described in connection with the embodiments disclosed herein may be implemented as electronic hardware, computer software, or combinations of both. To clearly illustrate this interchangeability of hardware and software, various illustrative components, blocks, modules, circuits, and steps have been described above generally in terms of their functionality.

The various illustrative logical blocks, modules, and circuits described in connection with the embodiments disclosed herein may be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

The steps of a method or algorithm described in connection with the embodiments disclosed herein may be embodied directly in hardware, in a software module executed by one or more processor(s), or in a combination of the two. A software module may reside in RAM memory, flash memory, ROM memory, EPROM memory, EEPROM memory, registers, hard disk, a removable disk, a CD-ROM, or any other form of storage medium known in the art. An exemplary storage medium is coupled to the processor such the processor can read information from, and write information to, the storage medium. In the alternative, the storage medium may be integral to the processor. The processor and the storage medium may reside in an ASIC. The ASIC may reside in a user terminal. In the alternative, the processor and the storage medium may reside as discrete components in a user terminal.

In accordance with the description herein, suitable computing devices include, by way of non-limiting examples, server computers, desktop computers, laptop computers, notebook computers, sub-notebook computers, netbook computers, netpad computers, set-top computers, media streaming devices, handheld computers. Internet appliances, mobile smartphones, tablet computers, personal digital assistants, video game consoles, and vehicles. Those of skill in the art will also recognize that select televisions, video players, and digital music players with optional computer network connectivity are suitable for use in the system described herein. Suitable tablet computers, in various embodiments, include those with booklet, slate, and convertible configurations, known to those of skill in the art.

In some embodiments, the computing device includes an operating system configured to perform executable instructions. The operating system is, for example, software, including programs and data, which manages the device's hardware and provides services for execution of applications. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. In some embodiments, the operating system is provided by cloud computing. Those of skill in the art will also recognize that suitable mobile smartphone operating systems include, by way of non-limiting examples, Nokia® Symbian® OS, Apple® iOS®, Research In Motion® BlackBerry OS®, Google® Android®, Microsoft® Windows Phone® OS, Microsoft® Windows Mobile® OS, Linux®, and Palm® WebOS®. Those of skill in the art will also recognize that suitable media streaming device operating systems include, by way of non-limiting examples, Apple TV®, Roku®, Boxee®, Google TV®, Google Chromecast®, Amazon Fire®, and Samsung® HomeSync®. Those of skill in the art will also recognize that suitable video game console operating systems include, by way of non-limiting examples, Sony® PS3®, Sony® PS4®, Microsoft® Xbox 360®. Microsoft Xbox One, Nintendo® Wii®, Nintendo® Wii U®, and Ouya®.

### Non-Transitory Computer Readable Storage Medium

In some embodiments, the platforms, systems, media, and methods disclosed herein include one or more non-transitory computer readable storage media encoded with a program including instructions executable by the operating system of an optionally networked computing device. In further embodiments, a computer readable storage medium is a tangible component of a computing device. In still further embodiments, a computer readable storage medium is optionally removable from a computing device. In some embodiments, a computer readable storage medium includes, by way of non-limiting examples, CD-ROMs, DVDs, flash memory devices, solid state memory, magnetic disk drives, magnetic tape drives, optical disk drives, distributed computing systems including cloud computing systems and services, and the like. In some cases, the program and instructions are permanently, substantially permanently, semi-permanently, or non-transitorily encoded on the media.

### Computer Program

In some embodiments, the platforms, systems, media, and methods disclosed herein include at least one computer program, or use of the same. A computer program includes a sequence of instructions, executable by one or more processor(s) of the computing device's CPU, written to perform a specified task. Computer readable instructions may be implemented as program modules, such as functions, objects, Application Programming Interfaces (APIs), computing data structures, and the like, that perform particular tasks or implement particular abstract data types. In light of the disclosure provided herein, those of skill in the art will recognize that a computer program may be written in various versions of various languages.

The functionality of the computer readable instructions may be combined or distributed as desired in various environments. In some embodiments, a computer program comprises one sequence of instructions. In some embodiments, a computer program comprises a plurality of sequences of instructions. In some embodiments, a computer program is provided from one location. In other embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof.

### Web Application

In some embodiments, a computer program includes a web application. In light of the disclosure provided herein, those of skill in the art will recognize that a web application, in various embodiments, utilizes one or more software frameworks and one or more database systems. In some embodiments, a web application is created upon a software framework such as Microsoft® .NET or Ruby on Rails (RoR). In some embodiments, a web application utilizes one or more database systems including, by way of non-limiting examples, relational, non-relational, object oriented, associative, and XML database systems. In further embodiments, suitable relational database systems include, by way of non-limiting examples, Microsoft® SQL Server, mySQL™, and Oracle®. Those of skill in the art will also recognize that a web application, in various embodiments, is written in one or more versions of one or more languages. A web application may be written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or extensible Markup Language (XML). In some embodiments, a web application is written to some extent in a presentation definition language such as Cascading Style Sheets (CSS). In some embodiments, a web application is written to some extent in a client-side scripting language such as Asynchronous Javascript and XML (AJAX), Flash® Actionscript, Javascript, or Silverlight®. In some embodiments, a web application is written to some extent in a server-side coding language such as Active Server Pages (ASP), ColdFusion®, Perl, Java™, JavaServer Pages (JSP), Hypertext Preprocessor (PHP), Python™, Ruby, Tcl, Smalltalk, WebDNA®, or Groovy. In some embodiments, a web application is written to some extent in a database query language such as Structured Query Language (SQL). In some embodiments, a web application integrates enterprise server products such as IBM® Lotus Domino®. In some embodiments, a web application includes a media player element. In various further embodiments, a media player element utilizes one or more of many suitable multimedia technologies including, by way of non-limiting examples, Adobe® Flash®, HTML 5, Apple® Quick Time®, Microsoft® Silverlight®, Java™, and Unity®.

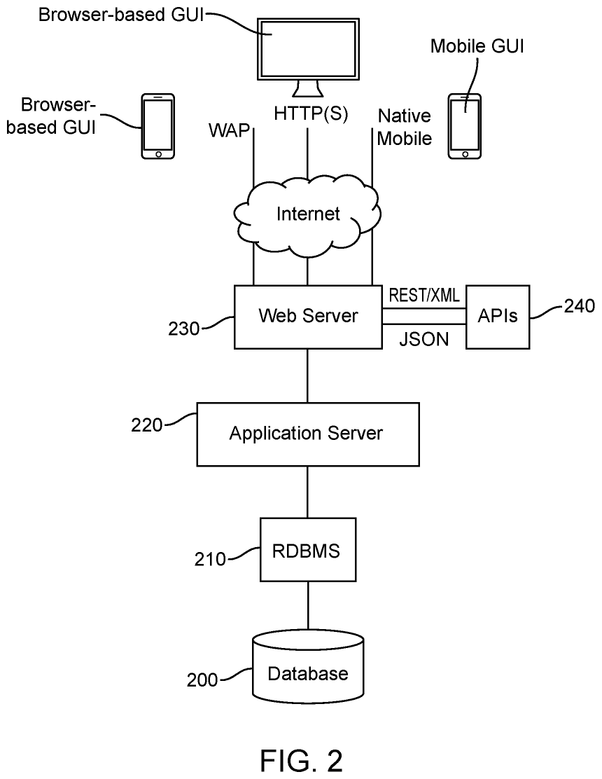

Referring to FIG. 2, in a particular embodiment, an application provision system comprises one or more databases 200 accessed by a relational database management system (RDBMS) 210. Suitable RDBMSs include Firebird, MySQL, PostgreSQL, SQLite, Oracle Database, Microsoft SQL Server, IBM DB2, IBM Informix, SAP Sybase, SAP Sybase, Teradata, and the like. In this embodiment, the application provision system further comprises one or more application severs 220 (such as Java servers, .NET servers, PHP servers, and the like) and one or more web servers 230 (such as Apache, IIS, GWS and the like). The web server(s) optionally expose one or more web services via app application programming interfaces (APIs) 240. Via a network, such as the Internet, the system provides browser-based and/or mobile native user interfaces.

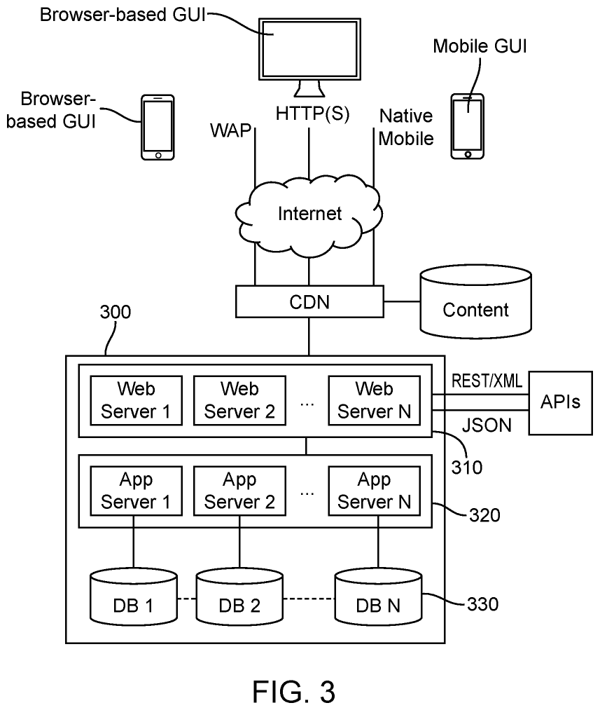

Referring to FIG. 3, in a particular embodiment, an application provision system alternatively has a distributed, cloud-based architecture 300 and comprises elastically load balanced, auto-scaling web server resources 310 and application server resources 320 as well synchronously replicated databases 330.

### Mobile Application

In some embodiments, a computer program includes a mobile application provided to a mobile computing device. In some embodiments, the mobile application is provided to a mobile computing device at the time it is manufactured. In other embodiments, the mobile application is provided to a mobile computing device via the computer network described herein.

In view of the disclosure provided herein, a mobile application is created by techniques known to those of skill in the art using hardware, languages, and development environments known to the art. Those of skill in the art will recognize that mobile applications are written in several languages. Suitable programming languages include, by way of non-limiting examples, C, C++, C#, Objective-C, Java™, Javascript, Pascal, Object Pascal, Python™, Ruby, VB .NET, WML, and XHTML/HTML with or without CSS, or combinations thereof.

Suitable mobile application development environments are available from several sources.

Commercially available development environments include, by way of non-limiting examples, Airplay SDK, alcheMo, Appcelerator®, Celsius, Bedrock, Flash Lite, .NET Compact Framework, Rhomobile, and WorkLight Mobile Platform. Other development environments are available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of skill in the art will recognize that several commercial forums are available for distribution of mobile applications including, by way of non-limiting examples, Apple® App Store, Google® Play, Chrome WebStore, Black Berry® App World, App Store for Palm devices, App Catalog for webOS, Windows® Marketplace for Mobile, Ovi Store for Nokia® devices, Samsung® Apps, and Nintendo® DSi Shop.

### Standalone Application

In some embodiments, a computer program includes a standalone application, which is a program that is run as an independent computer process, not an add-on to an existing process, e.g., not a plug-in. Those of skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB .NET, or combinations thereof. Compilation is often performed, at least in part, to create an executable program. In some embodiments, a computer program includes one or more executable complied applications.

### Web Browser Plug-In

In some embodiments, the computer program includes a web browser plug-in (e.g., extension, etc.). In computing, a plug-in is one or more software components that add specific functionality to a larger software application. Makers of software applications support plug-ins to enable third-party developers to create abilities which extend an application, to support easily adding new features, and to reduce the size of an application. When supported, plug-ins enable customizing the functionality of a software application. For example, plug-ins are commonly used in web browsers to play video, generate interactivity, scan for viruses, and display particular file types. Those of skill in the art will be familiar with several web browser plug-ins including, Adobe Flash Player, Microsoft Silverlight®, and Apple QuickTime®. In some embodiments, the toolbar comprises one or more web browser extensions, add-ins, or add-ons. In some embodiments, the toolbar comprises one or more explorer bars, tool bands, or desk bands.

In view of the disclosure provided herein, those of skill in the art will recognize that several plug-in frameworks are available that enable development of plug-ins in various programming languages, including, by way of non-limiting examples, C++, Delphi, Java™, PHP, Python™, and VB .NET, or combinations thereof.

Web browsers (also called Internet browsers) are software applications, designed for use with network-connected computing devices, for retrieving, presenting, and traversing information resources on the World Wide Web. Suitable web browsers include, by way of non-limiting examples, Microsoft Internet Explorer, Mozilla Firefox®, Google Chrome, Apple Safari®, Opera Software® Opera®, and KDE Konqueror. In some embodiments, the web browser is a mobile web browser. Mobile web browsers (also called microbrowsers, mini-browsers, and wireless browsers) are designed for use on mobile computing devices including, by way of non-limiting examples, handheld computers, tablet computers, netbook computers, subnotebook computers, smartphones, music players, personal digital assistants (PDAs), and handheld video game systems. Suitable mobile web browsers include, by way of non-limiting examples, Google Android® browser, RIM BlackBerry® Browser, Apple Safarik, Palm® Blazer, Palm® WebOS® Browser, Mozilla Firefox® for mobile, Microsoft Internet Explorer® Mobile, Amazon® Kindle® Basic Web, Nokia® Browser, Opera Software® Opera Mobile, and Sony PSP™ browser.

### Software Modules

In some embodiments, the platforms, systems, media, and methods disclosed herein include software, server, and/or database modules, or use of the same. In view of the disclosure provided herein, software modules are created by techniques known to those of skill in the art using machines, software, and languages known to the art. The software modules disclosed herein are implemented in a multitude of ways. In various embodiments, a software module comprises a file, a section of code, a programming object, a programming structure, or combinations thereof. In further various embodiments, a software module comprises a plurality of files, a plurality of sections of code, a plurality of programming objects, a plurality of programming structures, or combinations thereof. In various embodiments, the one or more software modules comprise, by way of non-limiting examples, a web application, a mobile application, and a standalone application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on a distributed computing platform such as a cloud computing platform. In some embodiments, software modules are hosted on one or more machines in one location. In other embodiments, software modules are hosted on one or more machines in more than one location.

### Databases

In some embodiments, the platforms, systems, media, and methods disclosed herein include one or more databases, or use of the same. In view of the disclosure provided herein, those of skill in the art will recognize that many databases are suitable for storage and retrieval of the information for determining a lung cancer-associated risk described herein. In various embodiments, suitable databases include, by way of non-limiting examples, relational databases, non-relational databases, object-oriented databases, object databases, entity-relationship model databases, associative databases, and XML databases. Further non-limiting examples include SQL, PostgreSQL, MySQL, Oracle, DB2, and Sybase. In some embodiments, a database is internet-based. In further embodiments, a database is web-based. In still further embodiments, a database is cloud computing-based. In a particular embodiment, a database is a distributed database. In other embodiments, a database is based on one or more local computer storage devices.

### Methods Utilizing a Computer

The methods and software described herein can utilize one or more computers. The computer can be used for managing customer and sample information such as sample or customer tracking, database management, analyzing molecular profiling data, analyzing cytological data, storing data, billing, marketing, reporting results, storing results, or a combination thereof. The computer can include a monitor or other graphical interface for displaying data, results, billing information, marketing information (e.g. demographics), customer information, or sample information. The computer can also include means for data or information input. The computer can include a processing unit and fixed or removable media or a combination thereof. The computer can be accessed by a user in physical proximity to the computer, for example via a keyboard and/or mouse, or by a user that does not necessarily have access to the physical computer through a communication medium such as a modem, an internet connection, a telephone connection, or a wired or wireless communication signal carrier wave. In some cases, the computer can be connected to a server or other communication device for relaying information from a user to the computer or from the computer to a user. In some cases, the user can store data or information obtained from the computer through a communication medium on media, such as removable media. It is envisioned that data relating to the methods can be transmitted over such networks or connections for reception and/or review by a party. The receiving party can be but is not limited to an individual, a health care provider or a health care manager. In one instance, a computer-readable medium includes a medium suitable for transmission of a result of an analysis of a biological sample. The medium can include a result of a subject, wherein such a result is derived using the methods described herein.

The entity obtaining the sample information can enter it into a database for the purpose of one or more of the following: inventory tracking, assay result tracking, order tracking, customer management, customer service, billing, and sales. Sample information can include, but is not limited to: customer name, unique customer identification, customer associated medical professional, indicated assay or assays, assay results, adequacy status, indicated adequacy tests, medical history of the individual, preliminary diagnosis, suspected diagnosis, sample history, insurance provider, medical provider, third party testing center or any information suitable for storage in a database. Sample history can include but is not limited to: age of the sample, type of sample, method of acquisition, method of storage, or method of transport.

The database can be accessible by a customer, medical professional, insurance provider, or other third party. Database access can take the form of digital processing communication such as a computer or telephone. The database can be accessed through an intermediary such as a customer service representative, business representative, consultant, independent testing center, or medical professional. The availability or degree of database access or sample information, such as assay results, can change upon payment of a fee for products and services rendered or to be rendered. The degree of database access or sample information can be restricted to comply with generally accepted or legal requirements for patient or customer confidentiality.

### Machine Learning

The systems, methods, software, and platforms as described herein can comprise computer-implemented methods of supervised or unsupervised learning methods, including SVM, random forests, clustering algorithm (or software module), gradient boosting, logistic regression, and/or decision trees. The machine learning methods as described herein can improve determination for a lung cancer-associated risk based on recording and analyzing any of the identifiers, lab results, patient outcomes, or any other relevant medical information as described herein. In some cases, the machine learning methods can intentionally group or separate the polymorphism profile or the treatment options. In some embodiments, some polymorphism profile or treatment options can be intentionally clustered or removed from any one phase of the plurality of phases of the medical care encounter.

Supervised learning algorithms can be algorithms that rely on the use of a set of labeled, paired training data examples to infer the relationship between an input data and output data. Unsupervised learning algorithms can be algorithms used to draw inferences from training data sets to output data. Unsupervised learning algorithms can comprise cluster analysis, which can be used for exploratory data analysis to find hidden patterns or groupings in process data. One example of an unsupervised learning method can comprise principal component analysis. Principal component analysis can comprise reducing the dimensionality of one or more variables. The dimensionality of a given variables can be at least 1, 5, 10, 50, 100, 200, 300, 400, 500, 600, 700, 800, 900, 1000, 1100, 1200 1300, 1400, 1500, 1600, 1700, 1800, or greater. The dimensionality of a given variables can be at most 1800, 1600, 1500, 1400, 1300, 1200, 1100, 1000, 900, 800, 700, 600, 500, 400, 300, 200, 100, 50, 10 or less.

The computer-implemented methods can comprise statistical techniques. In some embodiments, statistical techniques can comprise linear regression, classification, resampling methods, subset selection, shrinkage, dimension reduction, nonlinear models, tree-based methods, support vector machines, unsupervised learning, or any combination thereof.

A linear regression can be a method to predict a target variable by fitting the best linear relationship between a dependent and independent variable. The best fit can mean that the sum of all distances between a shape and actual observations at each point is the least. Linear regression can comprise simple linear regression and multiple linear regression. A simple linear regression can use a single independent variable to predict a dependent variable. A multiple linear regression can use more than one independent variable to predict a dependent variable by fitting a best linear relationship.

A classification can be a data mining technique that assigns categories to a collection of data in order to achieve accurate predictions and analysis. Classification techniques can comprise logistic regression and discriminant analysis. Logistic regression can be used when a dependent variable is dichotomous (binary). Logistic regression can be used to discover and describe a relationship between one dependent binary variable and one or more nominal, ordinal, interval or ratio-level independent variables. A resampling can be a method comprising drawing repeated samples from original data samples. A resampling can not involve a utilization of a generic distribution tables in order to compute approximate probability values. A resampling can generate a unique sampling distribution on a basis of an actual data. In some embodiments, a resampling can use experimental methods, rather than analytical methods, to generate a unique sampling distribution. Resampling techniques can comprise bootstrapping and cross-validation. Bootstrapping can be performed by sampling with replacement from original data and take “not chosen” data points as test cases. Cross validation can be performed by split training data into a plurality of parts.

A subset selection can identify a subset of predictors related to a response. A subset selection can comprise best-subset selection, forward stepwise selection, backward stepwise selection, hybrid method, or any combination thereof. In some instances, shrinkage fits a model involving all predictors, but estimated coefficients are shrunken towards zero relative to the least squares estimates. This shrinkage can reduce variance. A shrinkage can comprise ridge regression and a lasso. A dimension reduction can reduce a problem of estimating n+1 coefficients to a simpler problem of m+1 coefficients, where m<n. It can be attained by computing n different linear combinations, or projections, of variables. Then these n projections are used as predictors to fit a linear regression model by least squares. Dimension reduction can comprise principal component regression and partial least squares. A principal component regression can be used to derive a low dimensional set of features from a large set of variables. A principal component used in a principal component regression can capture the most variance in data using linear combinations of data in subsequently orthogonal directions. The partial least squares can be a supervised alternative to principal component regression because partial least squares can make use of a response variable in order to identify new features.

A nonlinear regression can be a form of regression analysis in which observational data are modeled by a function which is a nonlinear combination of model parameters and depends on one or more independent variables. A nonlinear regression can comprise a step function, piecewise function, spline, generalized additive model, or any combination thereof.

Tree-based methods can be used for both regression and classification problems. Regression and classification problems can involve stratifying or segmenting the predictor space into a number of simple regions. Tree-based methods can comprise bagging, boosting, random forest, or any combination thereof. Bagging can decrease a variance of prediction by generating additional data for training from the original dataset using combinations with repetitions to produce multistep of the same carnality/size as original data. Boosting can calculate an output using several different models and then average a result using a weighted average approach. A random forest algorithm can draw random bootstrap samples of a training set. Support vector machines can be classification techniques. Support vector machines can comprise finding a hyperplane that best separates two classes of points with the maximum margin. Support vector machines can constrain an optimization problem such that a margin is maximized subject to a constraint that it perfectly classifies data.

Unsupervised methods can be methods to draw inferences from datasets comprising input data without labeled responses. Unsupervised methods can comprise clustering, principal component analysis, k-Mean clustering, hierarchical clustering, or any combination thereof.

Use of absolute or sequential terms, for example, “will,” “will not,” “shall,” “shall not,” “must,” “must not,” “first,” “initially,” “next.” “subsequently,” “before,” “after,” “lastly,” and “finally,” are not meant to limit scope of the present embodiments disclosed herein but as exemplary.

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

As used herein, “or” may refer to “and”, “or,” or “and/or” and may be used both exclusively and inclusively. For example, the term “A or B” may refer to “A or B”, “A but not B”, “B but not A”, and “A and B”. In some cases, context may dictate a particular meaning.

Any systems, methods, software, and platforms described herein are modular. Accordingly, terms such as “first” and “second” do not necessarily imply priority, order of importance, or order of acts.

The term “about” when referring to a number or a numerical range means that the number or numerical range referred to is an approximation within experimental variability (or within statistical experimental error), and the number or numerical range may vary from, for example, from 1% to 15% of the stated number or numerical range. In examples, the term “about” refers to +10% of a stated number or value.

The terms “increased”, “increasing”, or “increase” are used herein to generally mean an increase by a statically significant amount. In some aspects, the terms “increased,” or “increase,” mean an increase of at least 10% as compared to a reference level, for example an increase of at least about 10%, at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, standard, or control. Other examples of “increase” include an increase of at least 2-fold, at least 5-fold, at least 10-fold, at least 20-fold, at least 50-fold, at least 100-fold, at least 1000-fold or more as compared to a reference level.

The terms “decreased”, “decreasing”, or “decrease” are used herein generally to mean a decrease by a statistically significant amount. In some aspects, “decreased” or “decrease” means a reduction by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g., absent level or non-detectable level as compared to a reference level), or any decrease between 10-100% as compared to a reference level. In the context of a marker or symptom, by these terms is meant a statistically significant decrease in such level. The decrease can be, for example, at least 10%, at least 20%, at least 30%, at least 40% or more, and is preferably down to a level accepted as within the range of normal for an individual without a given disease.

As used herein, the term “about” a number refers to that number plus or minus 10% of that number. The term “about” a range refers to that range minus 10% of its lowest value and plus 10% of its greatest value.

As used herein, the terms “treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit may be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

The terms “determining,” “measuring.” “evaluating.” “assessing.” “assaying.” and “analyzing” are often used interchangeably herein to refer to forms of measurement. The terms include determining if an element is present or not (for example, detection). These terms may include quantitative, qualitative or quantitative and qualitative determinations. Assessing may be relative or absolute. “Detecting the presence of” may include determining the amount of something present in addition to determining whether it is present or absent depending on the context.

A “subject” may be a biological entity containing expressed genetic materials. The biological entity may be a plant, animal, or microorganism, including, for example, bacteria, viruses, fungi, and protozoa. The subject may be tissues, cells and their progeny of a biological entity obtained in vivo or cultured in vitro. The subject may be a mammal. The mammal may be a human. The subject may be diagnosed or suspected of being at high risk for a disease. In some cases, the subject is not necessarily diagnosed or suspected of being at high risk for the disease. In some embodiments, the subject is a “patient”, who has been diagnosed with a disease (e.g., lung cancer).

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

### EXAMPLES

The following illustrative examples are representative of embodiments of the stimulation, systems, and methods described herein and are not meant to be limiting in any way.

### Example 1. Genetic Susceptibility to Lung Cancer and COPD in the National Lung Screening Trial: The Differing Role of Genes Conferring Risk in the Screening Setting

### Rationale

Recently, a risk-based approach to selecting lung cancer screening participants had been advocated where the risk of lung cancer was assessed by multivariate models such as the PLCOₘ₂₀₁₂. The PLCOₘ₂₀₁₂ model may predict the presence of chronic obstructive pulmonary disease (COPD). COPD is an underdiagnosed comorbid disease affecting 35% of NLST screening participants and increases lung cancer risk by 2-3 fold. However, compared to those with no COPD, COPD is associated with only one half the lung cancer mortality reduction reported in the NLST (15% vs 28%). This reduction in mortality benefit is because COPD is associated with more aggressive forms of cancer, lower surgical rates and greater deaths from other causes.

### Aim

Using 10,054 subjects from the NLST-ACRIN cohort, a sub-study of the NLST that recruited high risk smokers and followed them for an average of 6.4 years, pre-defined risk genotypes conferring susceptibility to COPD and/or lung cancer in a prospective study design were examined.

### Methods

Using prospective data from the NLST-ACRIN cohort, a 40 SNP panel using independent pre-defined risk genotypes including those identified from the genetic literature was genotyped. Risk genotypes increasing likelihood of lung cancer and compared outcomes according to the presence or absence of COPD were identified. A genetic risk score (GRS) and assessed its effect on lung cancer mortality were also derived.

### Results

12 risk genotypes conferring either an increased (n=6 susceptibility SNPs) or decreased (n=6 protective SNPs) risk of developing lung cancer were identified. After stratifying screening participants according to spirometric defined airflow limitation (COPD), differential effects of these risk genotypes on developing lung cancer with or without preexisting COPD (see Table 3) were found. In those with lung cancer and predominantly protective risk genotypes (low GRS), lung cancer mortality was significantly reduced relative to other deaths. Risk genotypes conferring risk of COPD and lung cancer conferred a greater lung cancer mortality.

### CONCLUSION

The genetic basis of lung cancer allowed for the differential effects of risk genotypes on the presence of COPD and of dying of lung cancer in the screening setting. Genetic variants identified in retrospective cross-sectional studies in a non-screening setting, without consideration of co-existing COPD, may not help identify who benefits from lung cancer screening.

TABLE 3
Differential effects of lung cancer risk genotypes according to
the presence of airflow limitation (COPD)

| | | Screening outcomes | | | | |
|---|---|---|---|---|---|---|
| Risk SNP | Phenotype | COPD | LC overall | LC + COPD | LC Alone | Dying of LC |
| CHRNA3/5 | Susceptible | ↑ | ↑ | ↑ | ↑↑ | ↑ |
| TERT | Susceptible | ↑ | ↑ | ↑ | ↑↑ | ↑ |
| BAT3 | Susceptible | | | | ↑ | |
| CYP2A6 | Susceptible | ↑ | ↑↑ | ↑ | ↑↑ | ↑↑ |
| IREB | Protective | ↓ | ↓↓ | ↓↓ | ↓↓ | ↓ |
| Fas I | Protective | | ↓↓ | ↓ | ↓↓ | ↓↓ |
| Rev 1 | Protective | | ↓↓ | ↓↓ | ↓↓ | ↓↓ |
| ITGA11 | Protective | | ↓ | | ↓ | |
| MMP12 | Protective | | ↓ | ↓ | ↓ | |
| IL-6 | Susceptible | | ↑ | ↑↑ | ↑ | |
| CRP | Protective | | | | ↓ | |
| AGER | Susceptible | | | | ↑↑ | |

### Example 2. Single Nucleotide Polymorphisms Associations with Chronic Obstructive Pulmonary Disease and Lung Cancer in the National Lung Screening Trial

Large cross-sectional genome wide association studies (GWAS) in Caucasians had reported several replicated associations between single nucleotide polymorphisms (SNPs) and lung cancer. Soon after the original GWAS SNP association studies for lung cancer were reported, it was shown that the chromosome 15q25 SNP variant, the strongest of the SNP associations, was also associated with chronic obstructive pulmonary disease (COPD). In that cross-sectional case-control study, the frequency of the nicotinic acetylcholine receptor subunit alpha 5 gene (CHNRA5) variant rs16969968 at the 15q25 locus was compared between healthy smokers (normal lung function), smokers with COPD, and smokers with lung cancer (with and without COPD). The groups were closely matched for age and smoking exposure history. This 15q25 association with both COPD and lung cancer had been extensively and independently replicated in subsequent GWAS or case-control studies. However, to date none of these lung cancer studies had examined SNP associations in a prospective cohort where the presence of underlying COPD, confirmed using baseline pulmonary function testing, has been considered. In the absence of sub-phenotyping for COPD, there may exist the potential for confounding or mediating effects underlying reported associations between SNP variants and lung cancer.

One way to explore the confounding or mediating effects of unrecognized COPD on the risk of developing lung cancer is through a large prospective study, where current or former smokers at risk of lung cancer are followed after their smoking history and lung function were documented at baseline. This reduces the biases that come from using lung cancer cases collected retrospectively (survivor bias); using lung function performed exclusively for pre-operative assessment (indication bias); or using smoking history collected after the diagnosis of lung cancer was made (recall bias). A prospective study design in the context of high risk smokers undergoing lung cancer screening also helps address the issue of bias stemming from a cross-sectional case-control study design where the presence of COPD has been largely ignored to date despite important effects on lung cancer outcomes and shortened life expectancy. In the context of lung cancer screening, it was shown in post-hoc analyses of the National Lung Screening Trial (NLST) that COPD not only increased the risk of developing lung cancer, but also of dying of lung cancer, in part due to more aggressive histology and reduced surgical rates. The NLST was a large prospective study of 53,000 high risk current and former smokers followed over a mean of 6.4 years. Among this cohort there were 9,367 Non-Hispanic whites from American College of Radiology, Imaging Network (ACRIN) arm of the study who underwent baseline pulmonary function tests and provided blood for DNA analysis. This group consisted of 5,793 healthy smokers, 3,018 with airflow limitation (33%) at baseline, and 380 lung cancers diagnosed during the follow up period. This study was undertook to better understand the possible mediating effect of pre-existing COPD on genetic associations in lung cancer. Preliminary results of this study have been reported in abstract form.

### Method

### Subject

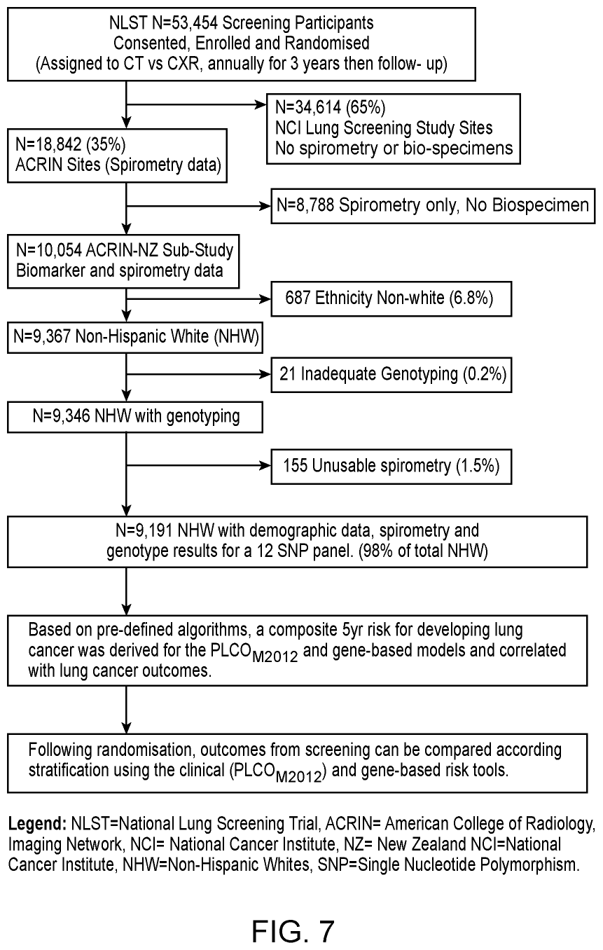

This was a secondary data analysis of the National Lung Screening Trial (NLST). The recruitment and study design of this trial, involving 53,452 screening participants yielding 2058 histology confirmed lung cancers. In the ACRIN sub-cohort of the NLST, participants from 23 centers agreed to undergo baseline pre-bronchodilator spirometry and, for a subgroup (FIG. 7), blood sampling for biomarker analysis (N=10,054). Demographic data, including history of diagnosed pre-morbid disease, were collected through an extensive questionnaire. From the total group (all ethnicities), data for Non-Hispanic whites (NHW) comprising 9,161 high risk smokers with available genotype data and information on COPD status (FIG. 7) was analyzed.

### Pulmonary Function Testing

In the NLST-ACRIN cohort, pre-bronchodilator spirometry was measured at baseline screening (TO) in the majority of participants meeting previously published criteria. The spirometry was measured by trained staff using a Spiropro spirometer (eResearchTechnology, GmbH, Germany). The severity of airflow limitation was defined according to the Global Initiative on Chronic Obstructive Lung Disease (GOLD) criteria grades 1-4. Total controls included those with airflow limitation (GOLD grade 1-4) while those with no airflow limitation were described as healthy controls (“Resistant Smokers”).

### Method: Lung Cancer Outcomes

Lung cancer cases included all those diagnosed during the trial (N=380), whether screen or non-screen detected (interval), or prevalent (diagnosed at TO or during the first year) or incident lung cancers (diagnosed during subsequent years T1 to T6) or at post-mortem. All lung cancer cases diagnosed during the trial (mean follow-up of 6.4 years) were confirmed on histological sampling according to accepted international classification criteria. Cause of death was a primary outcome for the NLST and was ascertained through review of clinical records and death certification.

### Method: Genotyping

Genomic DNA was extracted from buffy coat samples using standard salt-based methods and purified genomic DNA was aliquoted (10 ng/μL concentration) into 384-well plates. DNA concentration and purity were determined by using Nanodrop spectrophotometry. Samples were genotyped for 40 previously validated SNPs that had been identified in the literature as being associated with lung cancer or COPD (Table 9). Genotyping entailed using the Sequenom™ system (Sequenom™ Autoflex Mass Spectrometer and Samsung 24 pin nanodispenser) by Agena (Agena BioScience, San Diego, CA, USA) multiplexed into 2 assays (Agena MassARRAY Assay Design 3.0). The Sequenom™ sequences were designed in house by Agena with amplification and separation methods (iPLEX™) as previously described. Replication using this method was undertaken in 2,000 random samples and achieved 99% accuracy for genotyping. The forty independent SNPs had been validated in this New Zealand cohort of smokers with normal lung function, with spirometric-confirmed COPD and lung cancer (Table 8). Based on this study and those studies in the literature, the risk genotype/s were pre-specified and assigned as susceptible (Odds ratio >1.0) or protective (Odds ratio <1.0) for either COPD or lung cancer. For one SNP (AGER), opposing effects have been reported, so a two-sided analysis was undertook. A call rate for each SNP >95% was required.

### Statistical Analysis

Odds ratios with 95% Confidence Intervals (CI) and one-tailed mid-P exact tests were used to assess the magnitude and direction of the risk genotype association pre-defined for lung cancer. Statistical significance was defined as a one tailed P≤0.05 for comparisons between healthy controls and COPD, and P≤0.10 for comparisons with lung cancer or lung cancer with or without COPD (for reasons of powering and subsequent logistic regression modelling). One tailed P values were used as the risk genotypes were pre-specified based on earlier reported results (see above). All analyses were performed using SAS (V 9.4, SAS Institute Inc, Cary NC) or STATA statistical software (Release 16, College Station, TX: StataCorp LLC).

### Results

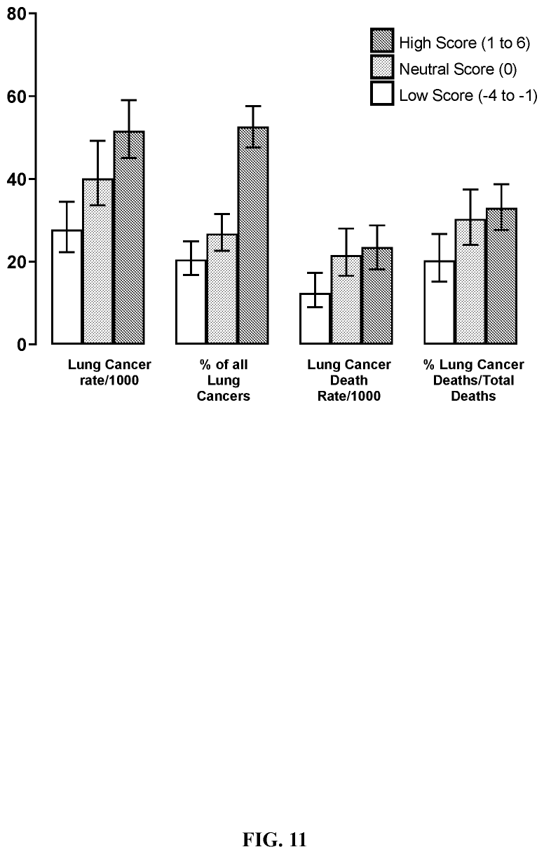

Genotype and lung function results were available for 9,191 subjects (98% of total NHW), with 20 excluded due to failed genotyping and 156 excluded due to unusable spirometry (FIG. 7). Based on baseline spirometry, 5,977 (65%) subjects had no airflow limitation and 3214 (35%) had airflow limitation. From these two groups 380 lung cancers (3.5%) were diagnosed prospectively with 184 in those with no airflow limitation (89.9 per 1000 person-years) and 196 in those with airflow limitation (97.9 per 1000 persons years) (FIG. 11). The genotype data for at least 35 of the 40 SNP variants was available for 8,947 subjects (97.3%) where call rates for each SNP exceeded 97%.

In Table 4, the demographic variables, pulmonary function tests and pre-morbid “doctor diagnosed” diseases were compared in the healthy controls (N=5793), those with pre-bronchodilator airflow limitation consistent with COPD (N=3018) and those with lung cancer (N=380, Table 4). Compared to those with no airflow limitation, screening participants who had airflow limitation (COPD) or developed lung cancer were: slightly older, more likely to be a current smoker, smoked for longer, quit smoking for less time, or had greater smoking duration and higher pack year exposure. Those with COPD or lung cancer also had a higher rate of “doctor diagnosed” COPD and lower BMI (Table 4). Clinical variables for those with lung cancer or COPD were largely comparable, including for “doctor diagnosed” COPD, although lung cancer cases were slightly older and smoked for longer (P<0.001). These similarities suggested just how closely a smoker's risk for COPD and lung cancer overlap according to historical variables. There was over-representation of “doctor diagnosed” COPD-related comorbidities (COPD, chronic bronchitis, emphysema, adult asthma and pneumonia) in those with airflow limitation (spirometric-defined COPD) or lung cancer (Table 4). However, of those with airflow limitation in these two groups, only 32% and 38% reported “doctor diagnosed” COPD respectively. Otherwise the 3 groups were similar in regards to other common pre-morbid conditions (e.g. cardiovascular diseases and cancer). Baseline pre-bronchodilator pulmonary function testing indicated that 52% of those who developed lung cancer had underlying airflow limitation consistent with COPD, which was significantly greater than the 34% in those who did not develop lung cancer (N=8,811; Mid-P Odds ratio=2.04, 95% CI 1.66-2.51, P<0.0001).

TABLE 4
Demographic and mortality data for NLST-ACRIN participants stratified according to
baseline airflow limitation (presence or absence) and subsequent lung cancer (mutually exclusive)

| | Healthy | | Lung | | | |
|---|---|---|---|---|---|---|
| | Controls | | Cancer | P value for | | |
| | (HC) | COPD | (LC) | pairwise comparisons ^{Φ} | | |
| | N = 5,793 | N = 3,018 | N = 380 | COPD | LC vs | LC vs |
| N = 9191† (% total) | (63.0%) | (32.8%) | (4.1%) | vs HC | HC | COPD |
| Demographics | | | | | | |
| Age (mean ± SD) | 61.18 ± 4.90 | 62.73 ± 5.23 | 63.96 ± 5.28 | <0.0001 | <0.0001 | <0.0001 |
| Male Gender | 3168 | 1845 (61.1%) | 213 (56.0%) | <0.0001 | 0.99 | 0.17 |
| | (54.7%) | | | | | |
| Current Smoker | 2588 | 1615 (53.5%) | 214 (56.3%) | <0.0001 | <0.0001 | 0.91 |
| | (44.7%) | | | | | |
| Pack Years (mean ± SD) | 53.7 ± 21.88 | 59.9 ± 24.76 | 63.6 ± 26.03 | <0.0001 | <0.0001 | 0.003 |
| Cigarettes/day (mean ± SD) | 27.90 ± 11.0 | 28.69 ± | 28.86 ± | 0.0002 | 0.07 | 0.99 |
| | | 11.09 | 10.89 | | | |
| Years Quit (mean ± SD) | 4.11 ± 5.26 | 3.15 ± 4.76 | 2.69 ± 4.49 | <0.0001 | <0.0001 | 0.46 |
| Smoking duration yrs (mean ± | 39.12 ± 7.30 | 42.10 ± 7.19 | 44.41 ± 7.18 | <0.0001 | <0.0001 | <0.0001 |
| SD) | | | | | | |
| Family history of Lung Cancer | 1378 | 711 (23.6%) | 101 (26.6%) | 0.99 | 0.65 | 0.58 |
| | (23.8%) | | | | | |
| Personal history of COPD# | 803 (13.9%) | 965 (32.0%) | 120 (31.6%) | <0.0001 | <0.0001 | 0.99 |
| BMI (mean ± SD) | 28.54 ± 5.15 | 26.80 ± 4.83 | 26.55 ± 4.51 | <0.0001 | <0.0001 | 0.95 |
| Education Level | 1566 | 949 (31.4%) | 109 (28.7%) | 0.0007 | 0.30 | 0.29 |
| High school or less | (27.0%) | 373 (12.4%) | 61 (16.1%) | | | |
| Post High school training | 729 (12.6%) | 648 (21.5%) | 89 (23.4%) | | | |
| Some College | 1366 | 513 (17.0%) | 53 (13.9%) | | | |
| College graduate | (23.6%) | 457 (15.1%) | 55 (14.5%) | | | |
| Postgraduate/Professional | 1006 | 78 (2.6%) | 13 (3.4%) | | | |
| Other/unknown | (17.4%) | | | | | |
| | 981 (16.9%) | | | | | |
| | 145 (2.5%) | | | | | |
| Lung Function | | | | | | |
| FEV₁ % predicted (mean ± | 89.32 ± | 66.92 ± | 73.91 ± | <0.0001 | <0.0001 | <0.0001 |
| SD) | 15.38 | 19.38 | 21.92 | | | |
| FEV₁/FVC (mean ± SD) | 77.23 ± 4.70 | 59.62 ± 9.12 | 66.35 ± | <0.0001 | <0.0001 | <0.0001 |
| | | | 11.63 | | | |
| % COPD (baseline spirometry) | 0% | 100% | 196 (52%) | na | na | na |
| Premorbid Disease | | | | | | |
| COPD# | 193 (3.3%) | 410 (13.6%) | 59 (15.5%) | <0.0001 | <0.0001 | 0.94 |
| Chronic Bronchitis | 517 (9.0%) | 454 (15.1%) | 52 (13.7%) | <0.0001 | 0.006 | 0.99 |
| Emphysema (Y on visual | 244 (4.2%) | 546 (18.1%) | 57 (15.0%) | <0.0001 | <0.0001 | 0.41 |
| assess) | | | | | | |
| Adult Asthma | 287 (5.0%) | 317 (10.5%) | 24 (6.3%) | <0.0001 | 0.72 | 0.03 |
| Pneumonia | 1509 | 947 (31.4%) | 124 (32.6%) | <0.0001 | 0.01 | 0.99 |
| | (26.05%) | | | | | |
| Heart Disease | 745 (12.9%) | 421 (13.9%) | 59 (15.5%) | 0.46 | 0.40 | 0.99 |
| Hypertension | 2056 | 1066 (35.3%) | 144 (37.9%) | 0.99 | 0.99 | 0.97 |
| | (35.5%) | | | | | |
| Stroke | 157 (2.7%) | 97 (3.2%) | 12 (3.2%) | 0.54 | 0.99 | 0.99 |
| Diabetes | 574 (9.9%) | 228 (7.6%) | 34 (8.9%) | 0.0008 | 0.99 | 0.99 |
| Any cancer History | 221 (3.8%) | 142 (4.7%) | 18 (4.7%) | 0.14 | 0.99 | 0.99 |

†Unknown COPD status N = 168 (9 Deaths);
‡ 14 lung cancers identified at post-mortem.
#Composite of ‘doctor-diagnosed’ COPD, emphysema, chronic bronchitis or adult asthma.
^{Φ} P-values from a Kruskall-wallis test for continuous variables and a chi-square test for categorical variables, with Bonferoni adjustment to account for pairwise comparisons.
na: not applicable.

After stratification of the lung cancer cases by the presence of airflow limitation (spirometry-defined COPD), those with lung cancer and pre-existing COPD were; more likely to be older with lower BMI, had greater smoking exposure, greater personal history of COPD or COPD-related comorbid diseases, or lower lung function (Table 5). Subjects with lung cancer and COPD also had more aggressive histological subtypes (small cell and non-small cell types) and less surgery for lung cancer despite comparable lung cancer staging (Table 5). Lung cancer prevalence was two-fold higher in those with pre-existing COPD (61.0/1000 vs 30.8/1000. P<0.001) but lung cancer mortality was unaffected by COPD status (P=0.444).

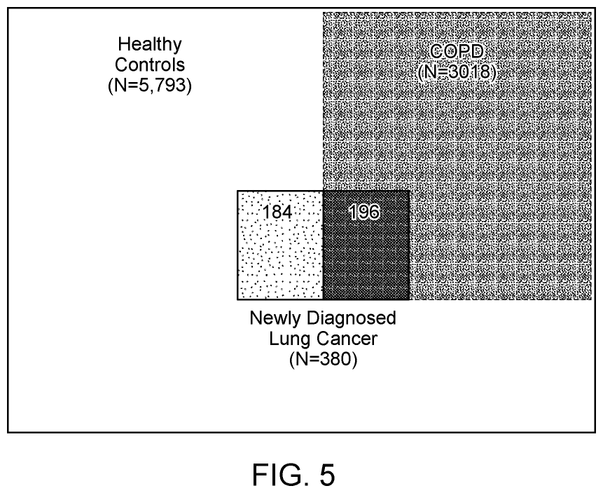

TABLE 5
Demographic and outcome data in lung cancer cases stratified by pre-existing COPD

| | Lung cancer cases stratified by COPD (see FIG. 5) | | | | |
|---|---|---|---|---|---|
| | Lung cancer | | Lung Cancer | | |
| | no COPD | | with COPD | | P value |
| N = 9191† (% total lung cancers) | N = 184/380 | (48.4%) | N = 196/380 | (51.6%) | <0.0001 |
| Lung cancer prevalence (per 1000) | 184/5977 | (30.8) | 196/3214 | (61.0) | |
| Demographics | | | | | |
| Age (mean ± SD)-yrs | 62.7 | ± 5.7 | 65.1 | ± 5.1 | <0.001 |
| Male Gender | 94 | (51.1%) | 119 | (60.7%) | 0.059 |
| Current Smoker | 95 | (51.6%) | 119 | (60.7%) | 0.074 |
| Pack Years (mean ± SD) | 60.3 | ± 23.8 | 66.7 | ± 27.7 | 0.007 |
| Cigarettes/day (mean ± SD) | 28.6 | ± 10.8 | 29.1 | ± 11.0 | 0.606 |
| Years Quit (mean ± SD) | 3.3 | ± 5.0 | 2.1 | ± 3.8 | 0.015 |
| Smoking duration years | 42.7 | ± 7.4 | 46.05 | ± 6.6 | <0.001 |
| (mean ± SD) | | | | | |
| Family history of Lung Cancer | 54 | (29.3%) | 47 | (24.0%) | 0.236 |
| Personal history of COPD# | 43 | (23.4%) | 77 | (39.3%) | 0.001 |
| BMI (mean ± SD) | 27.2 | ± 4.5 | 26.0 | ± 4.4 | 0.005 |
| Education Level | | | | | |
| High school/Post High School | 84 | (45.7%) | 86 | (43.9%) | 0.644 |
| training | | | | | |
| Some College/College graduate | 62 | (33.7%) | 80 | (40.8%) | |
| Postgraduate/Professional | 30 | (16.3%) | 25 | (12.8%) | |
| Lung Function/Airflow | | | | | |
| limitation | | | | | |
| FEV₁% predicted (mean ± SD) | 86.1 | ± 15.2 | 62.4 | ± 21.1 | <0.001 |
| FEV₁/FVC (mean ± SD) | 75.5 | ± 4.5 | 57.8 | ± 9.6 | <0.001 |
| GOLD 1-2 | — | | 139 | (71.9%) | na |
| GOLD 3-4 | — | | 57 | (29.1%) | na |
| Premorbid Disease | | | | | |
| (“doctor diagnosed”) | | | | | |
| COPD | 20 | (10.9%) | 39 | (19.9%) | 0.015 |
| Adult Asthma | 5 | (2.7%) | 19 | (9.7%) | 0.005 |
| Chronic Bronchitis | 20 | (10.9%) | 32 | (16.3%) | 0.122 |
| Emphysema | 16 | (8.7%) | 41 | (20.9%) | 0.001 |
| Pneumonia | 60 | (32.6%) | 64 | (32.7%) | 0.993 |
| Heart Disease | 28 | (15.2%) | 31 | (15.8%) | 0.872 |
| Hypertension | 69 | (37.5%) | 75 | (38.3%) | 0.878 |
| Diabetes | 19 | (10.3%) | 15 | (7.7%) | 0.362 |
| Any cancer History | 7 | (3.8%) | 11 | (5.6%) | 0.407 |
| Lung Cancer Histology (N = %) | | | | | |
| Small Cell | 20 | (10.9%) | 28 | (14.3%) | 0.032 |
| Squamous Cell | 41 | (22.3%) | 45 | (23.0%) | |
| Adenocarcinoma/BAC | 93 | (50.5%) | 72 | (36.7%) | |
| Non-small Cell | 20 | (10.9%) | 40 | (20.4%) | |
| Large cell/Other | 10 | (5.4%) | 11 | (5.6%) | |
| Lung Cancer Stage at Dx (N = %) | | | | | |
| Stage I-II | 89 | (48.4%) | 91 | (46.4%) | 0.488 |
| Stage III-IV | 88 | (47.8%) | 92 | (46.9%) | |
| Occult carcinoma/unknown | 7 | (3.8%) | 13 | (6.6%) | |
| Screen Detection | | | | | |
| T0-2 (Screening Interval) | 104 | (56.5%) | 125 | (63.8%) | 0.127 |
| T3-T7 (Follow-up Interval) | 80 | (43.5%) | 71 | (36.2%) | |
| Cancer Screen (N = %) | | | | | |
| Screen Detected | 89 | (48.4%) | 100 | (51.0%) | 0.170 |
| Missed/Interval | 15 | (8.2%) | 25 | (12.8%) | |
| Follow-up | 80 | (43.5%) | 71 | (36.2%) | |
| Surgery (N = % yes) | 105 | (57.1%) | 89 | (45.4%) | 0.023 |
| Lung Cancer Mortality | 85 | (46.2%) | 95 | (48.5%) | 0.657 |
| (N = total %) | | | | | |
| Lung Cancer Deaths (per 1000 | 89.9 | (72.7 to 111.3) | 97.9 | (79.7 to 119.1) | 0.444 ^{Φ} |
| person yrs, with 95% CI) | | | | | |

^{Φ} Adjusted for age and pack years

Risk genotype frequencies for this 40 unlinked SNPs (Table 8) were assessed and identified for 20 pre-defined risk genotypes that were associated with COPD, lung cancer or both (Table 9). These genotype frequencies accord with Hardy-Weinberg equilibrium and were consistent with the published frequencies in other Caucasian populations. In a univariate analysis, it was identified that eight SNP genotypes associated with spirometric-defined COPD alone (HHIP (n=2), GYPA, FAM13A, HTR4, DLC1, RIN3 and ADAM19), three SNP genotypes associated with both COPD and lung cancer (CHRNA5, TERT and IREB), and nine SNP genotypes associated with lung cancer alone (IL-6, AGER, BAT3, CYP2A6, FasL, Rev1, ITGA11, MMP12 and CRP, Table 6). Although the AGER risk genotype conferred increased risk of lung cancer, it was also associated with a reduced risk of COPD in a further analysis using a two-sided test (OR=0.78, 95% CI 0.66-0.91, P=0.002). For four of the SNP genotypes (CHRNA5, TERT, IREB and AGER), the risk genotype association with lung cancer and pre-existing COPD was reduced or lost (weaker and non-significant) suggesting a “Diluting” effect of COPD (Table 7). In these SNPs, the effect size was greater (and significant) in those with lung cancer alone, despite comparable sample size (powering). In contrast, the genetic association with lung cancer and pre-existing COPD was stronger for two SNP genotypes (IL-6 and Rev1) suggesting a “Enhancing” effect of COPD. For two risk genotypes (BAT3 and CRP), there was no apparent association with lung cancer overall, but after excluding those with pre-existing COPD, significant associations were found suggesting a “masking” effect from pre-existing COPD (Table 7).

TABLE 6
Odds ratios (OR) and Confidence Interval (CI) for risk genotypes according to lung cancer
cases and controls, with and without COPD (results from Table 9)

| | COPD | | | |
|---|---|---|---|---|
| | controls | | | Lung cancer |
| | (N = 3,018) vs | | | with COPD |
| Predefined | Healthy | Total Lung Cancer | Lung cancer alone | (N = 196) vs |
| Risk | Controls | (N = 380) vs Total | (N = 184) vs Healthy | COPD controls |
| genotypes* | (N = 5,793)‡ | Controls (N = 8,811)‡‡ | Controls (N = 5,793)‡‡ | (N = 3,018)‡‡ |
| COPD Alone | | | | |
| HHIP | OR = 0.88 | OR = 0.87) | OR = 1.04 | OR = 0.75 |
| (rs1489759) | (CI 0-0.97) | (CI 0-1.11 | (CI 0-1.44) | (CI 0-1.08) |
| GG (protective) | P = 0.017 | P = 0.19 | P = 0.60 | P = 0.099 |
| GYPA | OR = 0.87 | OR = 0.92 | OR = 1.02 | OR = 0.88 |
| (rs2202507) | (CI 0-0.95) | (CI 0-1.13) | (CI 0-1.35) | (CI 0-1.18) |
| GG (protective) | P = 0.004 | P = 0.27 | P = 0.55 | P = 0.24 |
| FAM13A | OR = 0.86 | OR = 1.06 | OR = 1.03 | OR = 1.14 |
| (rs7671167) | (CI 0-0.93) | (CI 0-1.28) | (CI 0-1.36) | (CI 0-1.50) |
| [symbol] otective | P = 0.001 | P = 0.68 | P = 0.58 | P = 0.78 |
| HTR4 | OR = 0.82 | OR = 1.03 | OR = 1.23 | OR = 0.91 |
| (rs11168048) | (CI 0-0.91) | (CI 0-1.30) | (CI 0-1.66) | (CI 0-1.28) |
| CC (protective) | P = 0.001 | P = 0.60 | P = 0.86 | P = 0.33 |
| DLC1 | OR = 1.14 | OR = 0.93 | OR = 0.89 | OR = 0.93 |
| (rs58863591) | (CI 1.05-∞) | (CI 0.77-∞) | (CI 0.67-∞) | (CI 0.72-∞) |
| CT/TT | P = 0.0028 | P = 0.74 | P = 0.77 | P = 0.68 |
| (susceptible) | | | | |
| RIN3 | OR = 0.89 | OR = 1.08 | OR = 1.07 | OR = 1.14 |
| (rs754388) | (CI 0-0.96) | (CI 0-1.30) | (CI 0-1.39) | (CI 0-1.46) |
| CG/GG | P = 0.007 | P = 0.77 | P = 0.68 | P = 0.79 |
| (protective) | | | | |
| HHIP | OR = 0.86 | OR = 0.87 | OR = 0.97 | OR = 0.82 |
| (rs13141641) | (CI 0-0.96) | (CI 0-1.11) | (CI 0-1.35) | (CI 0-1.17) |
| CC (protective) | P = 0.009 | P = 0.19 | P = 0.46 | P = 0.18 |
| ADAM19 | OR = 1.12 | OR = 0.85 | OR = 0.76 | OR = 0.89 |
| (rs1422795) | (CI 1.00-∞) | (0.64-∞) | (CI 0.49-∞) | (CI 0.61-∞) |
| CC | P = 0.046 | P = 0.83 | P = 0.85 | P = 0.68 |
| (susceptible) | | | | |
| [symbol] ancer and | | | | |
| COPD | | | | |
| [symbol] A5 (rs16969968) | OR = 1.19 | OR = 1.35 | OR = 1.43 | OR = 1.21 |
| [symbol] sceptible) | (CI 1.06-∞) | (CI 1.06-∞) | (CI 1.01-∞) | (CI 086-∞) |
| | P = 0.006 | P = 0.021 | P = 0.045 | P = 0.17 |
| [symbol] (rs402710) | OR = 1.12 | OR = 1.18 | OR = 1.32 | OR = 1.01 |
| [symbol] sceptible) | (CI 1.04-∞) | (CI 0.99-∞) | (CI 1.03-∞) | (CI 0.79-∞) |
| | P = 0.007 | P = 0.063 | P = 0.032 | P = 0.46 |
| [symbol] (rs2656069) | OR = 0.85 | OR = 0.78 | OR = 0.73 | OR = 0.88 |
| [symbol] (protective) | (CI 0-0.91) | (CI 0-0.93) | (CI 0-0.95) | (CI 0-1.13) |
| | P = 0.0002 | P = 0.012 | P = 0.023 | P-0.20 |
| [symbol] cancer alone | | | | |
| [symbol] s1800797) | OR = 1.02 | OR = 1.34 | OR = 1.06 | OR = 1.63 |
| [symbol] sceptible) | (CI 0.94-∞) | (CI 1.12-∞) | (CI 0.82-∞) | (CI 1.27-∞) |
| | P = 0.33 | P = 0.004 | P = 0.35 | P = 0.001 |
| [symbol] (rs2070600) | OR = 0.78 | OR = 1.40 | OR = 1.57 | OR = 1.33 |
| [symbol] (susceptible) | (0-0.89) | (CI 0-1.82) | (CI 0-2.23) | (CI 0-1.99) |
| | P = 0.99 | P = 0.024 | P = 0.022 | P = 0.13 |
| [symbol] (rs1052486) | OR = 0.91 | OR = 1.10 | OR = 1.25 | OR = 1.01 |
| [symbol] sceptible) | (CI 0.84-∞) | (CI 0.90-∞) | (CI 0.95-∞) | (CI 0.75-∞) |
| | P = 0.96 | P = 0.21 | P = 0.093 | P = 0.48 |
| [symbol] A6(rs7937) | OR = 1.08 | OR = 1.17* | OR = 1.19 | OR = 1.06 |
| [symbol] sceptible) | (CI 1.00-∞) | (CI 0.94-∞) | (CI 0.91-∞) | (CI 0.82-∞) |
| | P = 0.05 | P = 0.088 | P = 0.14 | P = 0.35 |
| [symbol] rs763110) | OR = 1.02 | OR = 0.77 | OR = 0.70 | OR-0.82 |
| [symbol] otective) | (CI 0-1.14) | (CI 0-1.00) | (CI 0-1.03) | (CI 0-1.18) |
| | P = 0.64 | P = 0.049 | P = 0.065 | P = 0.20 |
| [symbol] rs3087386) | OR = 1.07 | OR = 0.70 | OR = 0.78 | OR = 0.62 |
| otective) | (CI 0-1.16) | (CI 0-0.85) | (CI 0-1.03) | (CI 0-0.82) |
| | P = 0.91 | P = 0.001 | P = 0.071 | P = 0.002 |
| [symbol] 11 (rs2306022) | OR = 1.09 | OR = 0.76 | OR = 0.72 | OR = 0.78 |
| (protective) | (CI 0-1.21) | (CI 0-0.98) | (CI 0-1.04) | (CI 0-1.10) |
| | P = 0.93 | P = 0.039 | P = 0.074 | P = 0.12 |
| [symbol] 2 (rs645419) | OR = 0.99 | OR = 0.85 | OR = 0.88 | OR = 0.81 |
| [symbol] otective) | (CI 0-1.08) | (CI 0-1.04) | (CI 0-1.19) | (CI 0-1.10) |
| | P = 0.42 | P = 0.096 | P = 0.25 | P = 0.13 |
| [symbol] rs2808630) | OR = 1.01 | OR = 0.95 | OR = 0.66 | OR = 1.23 |
| [symbol] otective) | (CI 0-1.16) | (CI 0-1.31) | (CI 0-1.12) | (CI 0-1.84) |
| | P = 0.56 | P = 0.42 | P = 0.099 | P = 0.80 |

OR = odds ratio, P = P value, Chi Square (odds ratios) and P values are from a Mid-P exact (1 tailed). ‡Threshold for statistical significance defined as P ≤ 0.05, ‡‡Threshold for statistical significance defined as P ≤ 0.10, *Significantly different to healthy controls [symbol]
indicates data missing or illegible when filed

TABLE 7
Differential effect of COPD status on risk genotypes on the
development of lung cancer (data from Table 6 and Table 9)

| | | Risk genotype effects on significant (bold) | | | | |
|---|---|---|---|---|---|---|
| | | odds ratios for COPD and/or lung cancer | | | | |
| | | | Total | LC | LC + | Possible |
| | | | LC | Alone | COPD | modifying |
| | Risk | COPD | vs | vs | vs | effect of |
| Risk SNP | Genotype | vs HC | TC | HC | COPD | COPD |
| COPD and Lung | | | | | | |
| cancer | | | | | | |
| CHRNA5 | AA | 1.19 ↑ | 1.35 | 1.43 | 1.21 | Diluting |
| | Susceptible | | ↑↑ | ↑↑ | | |
| TERT | CC | 1.12 ↑ | 1.18 | 1.32 | 1.02 | Diluting |
| | Susceptible | | ↑ | ↑↑ | | |
| IREB | CC/CT | 0.85 ↓ | 0.78 | 0.73 | 0.95 | Diluting |
| | Protective | | ↓↓ | ↓↓ | | |
| Lung cancer alone | | | | | | |
| AGER | TT/TC | 0.78^{‡} | 1.40 | 1.57 | 1.33 | Diluting |
| | Susceptible | | ↑↑ | ↑↑ | | |
| BAT3 | GG | 0.91 | 1.1 | 1.25 ↑ | 1.01 | Masking |
| | Susceptible | | | | | |
| IL-6 | GG | 1.02 | 1.34 | 1.06 | 1.63 | Enhancing |
| | Susceptible | | ↑↑ | | ↑↑↑ | |
| CYP2A6 | TT | 1.08 ↑ | 1.17 | 1.19 | 1.06 | Possible |
| | Susceptible | | ↑ | | | diluting |
| Fas I | TT | 1.02 | 0.77 | 0.7 | 0.82 | Possible |
| | Protective | | ↓↓ | | | diluting |
| ITGA11 | TT/TC | 1.09 | 0.76 | 0.72 | 0.78 | Minimal |
| | Protective | | ↓↓ | | | effect |
| MMP12 | GG | 0.99 | 0.85 ↓ | 0.88 | 0.81 | Minimal |
| | Protective | | | | | effect |
| Rev 1 | GG | 1.07 | 0.70 | 0.78 ↓ | 0.62 | Enhancing |
| | Protective | | ↓↓ | | ↓↓↓ | |
| CRP | CC | 1.01 | 0.95 | 0.66 | 1.23 | Masking |
| | Protective | | | ↓↓↓ | | |

Arrow key for odds ratios (bold indicating significance),
^{‡}Risk genotype confers opposing effects-COPD protective and Lung cancer susceptible (28). Susceptible 1.00-1.24 ↑, 1.25-1.49 ↑↑, 1.50 upwards ↑↑↑↑: Protective 0.85-1.00 ↓, 0.70-0.84 ↓↓, up to 0.69 ↓↓↓
Legend:
HC = Healthy Controls,
TC = Total Controls (see Table 6)

TABLE 8
Risk genotypes, associations and call rates for single nucleotide polymorphisms (SNPs)
tested for validation from past GWAS and candidate gene studies

| | | | ^{‡}Risk | | | Validation | Call |
|---|---|---|---|---|---|---|---|
| NP# | Gene | Rs | genotype | Effect | Disease | studies | rate |
| 1 | CHRNA5 | Rs16969968 | AA | susceptible | LC/COPD | LC/COPD GWAS | 99.2% |
| 2 | CYP2E1 | Rs2031920 | TT/TC | susceptible | LC | Candidate, 20 | 99.2% |
| | | | | | | SNP | |
| 3 | IL-18 | Rs360721 | CC | susceptible | LC | Candidate, 20 | 98.7% |
| | | | | | | SNP | |
| 4 | IL-8 | Rs4073 | TT | susceptible | LC | Candidate, 20 | 99.0% |
| | | | | | | SNP | |
| 5 | IL-1B | Rs16944 | GG | susceptible | LC | Candidate, 20 | 98.7% |
| | | | | | | SNP | |
| 6 | ITGA11 | Rs2306022 | TT/TC | protective | LC | Candidate, 20 | 98.1% |
| | | | | | | SNP | |
| 7 | NAT-2 | Rs1799930 | GG | susceptible | LC | Candidate, 20 | 99.1% |
| | | | | | | SNP | |
| 8 | 1-ACT | Rs4934 | GG | susceptible | LC | Candidate, 20 | 98.7% |
| | | | | | | SNP | |
| 9 | Cerberus 1 | Rs10115703 | AA/AG | susceptible | LC | Candidate, 20 | 99.4% |
| | | | | | | SNP | |
| 10 | DAT1 | Rs6413429 | GT/TT | susceptible | LC | Candidate, 20 | 99.2% |
| | | | | | | SNP | |
| 11 | TNFR1 | Rs1139417 | AA | susceptible | LC | Candidate, 20 | 98.9% |
| | | | | | | SNP | |
| 12 | TLR9 | Rs5743836 | CC | susceptible | LC | Candidate, 20 | 99.2% |
| | | | | | | SNP | |
| 13 | P73 | Rs2273953 | CC | protective | LC | Candidate, 20 | 98.4% |
| | | | | | | SNP | |
| 14 | SOD3 | Rs1799895 | GG/GC | protective | LC | Candidate, 20 | 95.2% |
| | | | | | | SNP | |
| 15 | ITGB3 | Rs2317676 | GG/GA | protective | LC | Candidate, 20 | 98.7% |
| | | | | | | SNP | |
| 16 | DRD2 | Rs1799732 | CDl/DlDl | protective | LC | Candidate, 20 | 99.3% |
| | | | | | | SNP | |
| 17 | BCL2 | Rs2279115 | AA | protective | LC | Candidate, 20 | 97.4% |
| | | | | | | SNP | |
| 18 | XPD | Rs13181 | GG | protective | LC | Candidate, 20 | 99.0% |
| | (ERCC2) | | | | | SNP | |
| 19 | Rev1 | Rs3087386 | CC | protective | LC | Candidate, 20 | 97.8% |
| | | | | | | SNP | |
| 20 | FasL | Rs763110 | TT | protective | LC | Candidate, 20 | 99.1% |
| | | | | | | SNP | |
| | Additional SNPs from GWAS and meta-analyses | | | | | | |
| 21 | BAT3 | Rs1052486 | GG | susceptible | LC | LC GWAS, 9 SNP | 98.5% |
| 22 | TERT | Rs 402710 | GG | susceptible | LC | LC GWAS, 9 SNP | 98.8% |
| 23 | CRP | Rs2808630 | CC | protective | LC | LC GWAS, 9 SNP | 99.2% |
| 24 | FAM13A | Rs7671167 | CC | protective | COPD | COPD GWAS, 9 | 99.0% |
| | | | | | | SNP | |
| 25 | HHIP | Rs1489759 | GG | protective | COPD | COPD GWAS, 9 | 99.1% |
| | | | | | | SNP | |
| 26 | ADAM19 | Rs1422795 | CC | susceptible | COPD | COPD GWAS, 9 | 98.9% |
| | | | | | | SNP | |
| 27 | AGER | Rs2070600 | TT/TC | susceptible | COPD/LC | COPD GWAS, 9 | 98.9% |
| | | | | | | SNP | |
| 28 | GYPA | Rs2202507 | CC | protective | COPD | COPD GWAS, 9 | 99.1% |
| | | | | | | SNP | |
| 29 | GRP126 | Rs11155242 | CC | susceptible | COPD | COPD GWAS, 9 | 98.5% |
| | | | | | | SNP | |
| 30 | GSTCD | Rs10516526 | GG/AG | susceptible | COPD | COPD GWAS | 98.5% |
| 31 | IREB | Rs2656069 | AG/GG | protective | COPD | COPD GWAS | 98.9% |
| 32 | HTR4 | Rs11168048 | CC | protective | COPD | COPD GWAS | 98.5% |
| 33 | RIN3 | Rs754388 | CG/GG | protective | COPD | COPD GWAS | 98.9% |
| 34 | ADCY2 | Rs11134242 | TT | susceptible | COPD | COPD GWAS | 98.6% |
| 35 | MMP12 | Rs645419 | GG | protective | LC/COPD | Meta-Analysis | 98.7% |
| 36 | CYP2A6 | Rs7937 | TT | susceptible | COPD | COPD GWAS | 98.7% |
| 37 | DLC1 | Rs58863591 | CC/CT | susceptible | COPD | COPD GWAS | 99.0% |
| 38 | HHIP | Rs1542725 | GG | susceptible | COPD | COPD GWAS | 98.3% |
| 39 | IL-6 | Rs1800797 | GG | susceptible | LC | LC GWAS | 98.4% |
| 40 | HHIP | Rs13141641 | CC | protective | COPD | COPD GWAS | 98.5% |

^{‡}A genotype (recessive or co-dominant model) approach was chosen to assigning risk based on published results and statistical recommendation

TABLE 9
Frequency of risk genotypes according to clinical phenotype where lung cancer cases and
controls were sub-phenotyped by spirometric-defined COPD (GOLD 1-4)

| | Controls | | | | | |
|---|---|---|---|---|---|---|
| | Total | Healthy | | Lung Cancer | | |
| Predefined | controls | controls | COPD | Total LC | LC Alone | LC + COPD |
| Risk genotypes^{#} | N = 8,811 | N = 5,793 | N = 3,018 | N = 380 | N = 184 | N = 196 |
| COPD Alone | | | | | | |
| HHIP | 1419/8729 | 968/5741 | 451/2988 | 55/379 | 32/183 | 23/196 |
| (rs1489759) | (16.3%) | (16.9%) | (15.1%) | (14.5%) | (17.5%) | (11.7%) |
| GG (protective) | | | | | | |
| % GG | | | | | | |
| GYPA | 2,074/8,727 | 1413/5733 | 661/2994 | 85/380 | 46/184 | 39/196 |
| (rs2202507) | (23.8%) | (24.6%) | (22.1%) | (22.4%) | (25.0%) | (19.9%) |
| GG (protective) | | | | | | |
| % GG | | | | | | |
| FAM13A | 2,238/8,718 | 1528/5728 | 710/2990 | 101/378 | 50/183 | 51/195 |
| (rs7671167) | (25.7%) | (26.7%) | (23.7%) | (26.7%) | (27.3%) | (26.2%) |
| CC (protective) | | | | | | |
| % CC | | | | | | |
| HTR4 | 1485/8,680 | 1029/5,709 | 456/2971 | 65/370 | 38/179 | 27/191 |
| (rs11168048) | (17.1%) | (18.0%) | (15.3%) | (17.6%) | (21.2%) | (14.1%) |
| CC (protective) | | | | | | |
| % CC | | | | | | |
| DLC1 | 3101/8725 | 1980/5736 | 1121/2989 | 125/369 | 56/176 | 69/193 |
| (rs58863591) | (35.5% | (34.5%) | (37.5%) | (33.9%) | (31.8%) | (35.8%) |
| CT/TT | | | | | | |
| (susceptible) | | | | | | |
| % CT/TT | | | | | | |
| RIN3 (rs754388) | 2974/8718 | 2007/5733 | 967/2985 | 134/373 | 66/180 | 68/193 |
| CG/GG | (34.1%) | (35.0%) | (32.4%) | (35.9%) | (36.7%) | (35.2%) |
| (protective) | | | | | | |
| % CG/GG | | | | | | |
| HHIP | 1420/8683 | 973/5714 | 447/2969 | 54/370 | 30/180 | 24/190 |
| (rs13141641) | (16.4%) | (17.0%) | (15.1%) | (14.6%) | (16.7%) | (12.6%) |
| CC (protective) | | | | | | |
| % CC | | | | | | |
| ADAM19 | 1062/8711 | 674/5730 | 388/2981 | 40/379 | 17/184 | 23/195 |
| (rs1422795) | (12.2%) | (11.8%) | (13.0%) | (10.6%) | (9.2%) | (11.8%) |
| CC (susceptible) | | | | | | |
| % CC | | | | | | |
| COPD and LC | | | | | | |
| CHRNA5 | 1065/8738 | 663/5743 | 402/2995 | 60/380 | 29/184 | 31/196 |
| (rs16969968) | (12.2%) | (11.5%) | (13.4%) | (15.8%) | (15.8%) | (15.8%) |
| AA (susceptible) | | | | | | |
| % AA | | | | | | |
| TERT (rs402710) | 3782/8700 | 2433/5721 | 1349/2979 | 179/377 | 90/182 | 89/195 |
| CC (susceptible) | (43.4%) | (42.5%) | (45.3%) | (47.5%) | (49.5%) | (45.6%) |
| % CC | | | | | | |
| IREB | 3316/8721 | 2257/5734 | 1059/2987 | 120/372 | 57/178 | 63/194 |
| (rs2656069) | (38.0%) | (39.4%) | (35.5%) | (32.3% | (32.0%) | (32.5%) |
| CC/CT | | | | | | |
| (protective) | | | | | | |
| % CC/CT | | | | | | |
| Lung cancer alone | | | | | | |
| CYP2A6 | 2643/8700 | 1705/5720 | 938/2980 | 123/371 | 60/179 | 63/192 |
| (rs7937) | (30.4%) | (29.8%) | (31.5%) | (33.2%) | (33.5%) | (32.8%) |
| TT (susceptible) | | | | | | |
| % TT | | | | | | |
| BAT3 | 2100/8676 | 1414/5707 | 686/2969 | 98/376 | 53/182 | 45/194 |
| (rs1052486) | (24.2%) | (24.8%) | (23.1%) | (26.1%) | (29.1%) | (23.2%) |
| GG (susceptible) | | | | | | |
| % GG | | | | | | |
| AGER | 771/8715 | 546/5735 | 225/2980 | 45/377 | 26/183 | 19/194 |
| (rs2070600) | (8.8%) | (9.5%) | (7.6%) | (11.9%) | (14.2%) | (9.8%) |
| TT/TC | | | | | | |
| (susceptible) | | | | | | |
| % TT/TC | | | | | | |
| ITGA11 | 1414/8640 | 907/5689 | 507/2951 | 49/377 | 22/183 | 27/194 |
| (rs2306022) | (16.4%) | (15.9%) | (17.2%) | (13.0%) | (12.0) | (13.9%) |
| TT/TC | | | | | | |
| (protective) | | | | | | |
| % TT/TC | | | | | | |
| CRP (rs2808630) | 698/8737 | 457/5743 | 241/2994 | 29/379 | 10/184 | 19/195 |
| CC (protective) | (8.0%) | (8.0%) | (8.0%) | (7.7%) | (5.4%) | (9.7%) |
| % CC | | | | | | |
| MMP12 | 2085/8693 | 1376/5722 | 709/2971 | 79/375 | 40/183 | 39/192 |
| (rs645419) | (24.0%) | (24.0%) | (23.9%) | (21.1%) | (21.9%) | (20.3%) |
| GG (protective) | | | | | | |
| % GG | | | | | | |
| FasL (rs763110) | 1254/8729 | 819/5739 | 435/2990 | 43/378 | 19/183 | 24/195 |
| TT (protective) | (14.4%) | (14.3%) | (14.6%) | (11.4%) | (10.4%) | (12.3%) |
| % TT | | | | | | |
| Rev1 | 2701/8610 | 1749/5662 | 952/2948 | 91/376 | 47/182 | 44/194 |
| (rs3087386) | (31.4%) | (30.9%) | (32.3%) | (24.2%) | (25.8%) | (22.7%) |
| GG (protective) | | | | | | |
| % GG | | | | | | |
| IL-6 (rs1800797) | 3060/8682 | 2003/5709 | 1057/2973 | 155/368 | 65/178 | 90/190 |
| GG (susceptible) | (35.2%) | (35.1%) | (35.6%) | (42.1%) | (36.5%) | (47.4%) |
| % GG | | | | | | |

^{#}Genotype frequencies where failed genotypes have been excluded

### Discussion

In a post-hoc analysis of nearly 10,000 non-Hispanic white participants of the NLST, it was found that several previously validated SNP variants were associated with the development of lung cancer (N=12). These pre-defined risk genotypes were associated with lung cancer in high risk current and former smokers with and without spirometry-based airflow limitation consistent with COPD. It was found that while CHRNA5 (AA) and TERT (CC) risk genotypes conferred an increased risk for both lung cancer and COPD, the risk for lung cancer was greater in those with no pre-existing COPD (e.g., possible diluting effect from COPD). Similarly, it was found that the IREB (CC/CT) risk genotype conferred lower risk of COPD and lung cancer, particularly in those with no COPD (diluting effect from COPD). In contrast, the increased risk of lung cancer associated with the IL-6 (GG) risk genotype and reduced risk from the Rev1 (GG) genotype, was confined to those with underlying COPD (enhancing effect from COPD). The protective effect of CRP (CC) risk genotype and susceptibility effect of BAT3 (GG) genotype, was only evident in that developing lung cancer with no pre-existing COPD (masking effect from COPD). These results suggested that underlying COPD may directly or indirectly mediate (enhance, dilute or mask) the risk association with lung cancer. Although 8 COPD-related SNPs were validated in this NLST sub-study, these findings are the subject of further analyses. These results supported that genetic susceptibility to lung cancer may involve a complex interplay with genetic susceptibility to COPD.

One aspect of this study was the examination of risk genotypes for lung cancer in the context of pre-existing COPD. In a case-control study, it was shown that the CHRNA5 AA genotype (rs16969968) increased the risk of both COPD and lung cancer. This dual action had been replicated and had also shown that this CHRNA5 variant increased smoking exposure independently through nicotine addiction. In the current cohort study, it was confirmed this finding and showed that a similar dual action is possible for a TERT CC genotype (rs402710) and IREB CC/TT genotypes (rs2656069). However, for all 3 risk genotypes were reported for the first time that pre-existing COPD appeared to weaken these associations. The genetic susceptibility of lung cancer might also involve genes conferring risk of COPD was suggested previously but had been inadequately addressed. This was because in most lung cancer genetic studies there is no lung function results available with which to assess for mediating effects and overlapping associations. The CHRNA5 locus had also been associated with smoking addiction best demonstrated by daily cigarette consumption where there was a consistently reported finding of increased cigarettes per day for those with the AA susceptibility genotype. In a mediation analysis, it had recently shown this variant has independent effects on cigarette consumption. COPD and lung cancer. It was also found that while the AGER TT/TC genotype was associated with an increased risk for lung cancer, it conferred a decreased risk for COPD. This opposing effect has been described herein, where the association with lung cancer is somewhat dependent on the prevalence of COPD. Accordingly, the genetic susceptibility to lung cancer overlaps with that of COPD.

A second feature of this study was the use of “healthy” smokers as controls in confirming genetic associations with lung cancer. While this has been adopted in studies for COPD, it had not been a feature of lung cancer association studies. Traditional case-control studies for lung cancer were potentially subject to confounding or mediating effects from unrecognised associations with COPD rather than lung cancer itself. This is because COPD is present in about 20-30% of high-risk smokers and 50-70% of those developing lung cancer. As the prevalence of COPD in the NLST cohorts was 34% in those with no cancer (total controls) and 51% in those with lung cancer, confounding by COPD is a possibility in lung cancer studies. A potential example of confounding by COPD is reported in one lung cancer GWAS, where the association of the GYPA/HHIP locus with lung cancer may, according to the findings, be due to its association with COPD (Table 6). The reason healthy controls may provide an alternate “control” from which to identify genetic associations in lung cancer is to identify the mediating effects of COPD and because genes conferring a reduced risk (protective) may be of greater relevance than so called susceptibility genes. Five of the nine SNP associations it was found that for lung cancer alone, where no association with COPD was evident, conferred a lower risk for lung cancer (ITGA11, CRP, Rev1, MMP12 and FasL). It is important to capture these genetic contributors to risk as they might best reflect why some smokers do not get COPD or lung cancer. The identification of protective SNPs associated with “resistant” smokers may also be an important component of future risk testing for outcomes from lung cancer, where the response to smoking and variation in airway biology determine outcomes (discussed further below).

Another feature of this study was the identification of SNP associations with lung cancer in the absence of COPD. This is important because unrecognised COPD may have a mediating effect on genetic associations for lung cancer and it was found that over 60% of those with spirometry-defined COPD are not clinically diagnosed (unrecognised). This can only be identified where spirometry has been measured in a prospective study of high-risk smokers to avoid confounding or mediating effects from pre-existing COPD (see above). It was noted that the prevalence of COPD in case-series of lung cancer is generally not reported but very dependent on how the lung cancer cases and controls were identified. Based on these results, this has a major impact on the direction and strengths of associations found with lung cancer. It is likely that large cross-sectional case-studies may have a higher prevalence of adenocarcinoma as this subgroup generally do better and more likely to appear in retrospective studies. For the Rev1 and IL-6 risk genotypes, the association with lung cancer (protective and susceptible respectively) was most evident for those with co-existing COPD but not those with COPD controls. No genotype effect on lung cancer histology was seen in this study. This suggests that there exist SNP associations that are best found in lung cancer with and without pre-existing COPD due to the mediating effects of COPD. This might also have important implications with regards to lung cancer biology, as pre-existing COPD has been shown to be associated with more aggressive forms of lung cancer with regards to histology (more Small cell and Non-small cell subtypes with less adenocarcinoma) and reduced growth rate (lower volume doubling times). Differences in biology of this type may affect treatment responsiveness or attenuate the utility of lung cancer screening. In this regard, genetic variants conferring risk of lung cancer in those without COPD is of interest because this group has been shown to do better with screening compared to those with underlying COPD. While many reasons for this observation exists, one possibility is that lung cancer developing in the setting of COPD may be more difficult to identify during screening or more likely to be found as interval cancers. COPD is also associated with less surgery, greater non-lung cancer mortality and shorter life expectancy. For these reasons, those most likely to benefit from lung cancer screening might be found among those with normal lung function or mild-moderate COPD, as their lung cancers are more amenable to detection by CT-based screening and curative treatment by surgery.

These results highlight the complex interplay between two smoking-related diseases and genetic susceptibility. Not only were genetic variants confirmed to be associated with more than one outcome, it was shown that in this study that subgroups of lung cancer patients, in this case those with normal lung function, had somewhat different genetic susceptibility. Several SNP associations with COPD were also replicated. Importantly, the ability to sub-phenotype those with lung cancer into those with and without pre-existing COPD has enabled identifying genetic associations not evident in the whole group (BAT3 and CRP) exemplifying potential masking (confounding) effects. This was of interest as the outcomes for those diagnosed with lung cancer may be strongly affected by the presence of underlying COPD. In conclusion, a number of SNP variants were identified to confer an increased or decreased risk of COPD alone and lung cancer alone and the combination of the two. The interplay between high risk smokers with and without COPD was taken into account for recognising the important effect this comorbid disease has on the development of lung cancer in a screening context.

### Example 3. Polygenic Risk Score Identifies More Lethal Lung Cancers in the National Lung Screening Trial: A Prospective Clinical Validity Study

Lung cancer is believed to result in the main from the combined effect of genetic susceptibility and exposure to smoking or other aero pollutants. Epidemiological studies have identified a number of clinical risk variables underlying lung cancer susceptibility and these have been combined in models such as the PLCO_{M2012} to derive a composite risk for future lung cancer. These risk models have been validated in independent cohorts to a variable degree and performance characteristics. Numerous studies have identified single nucleotide polymorphisms (SNPs) that are consistently associated with lung cancer although some methodological issues exist in these studies. This includes cross-sectional study design with the potential for confounding effects and survival bias. None of these genetic studies have validated their risk SNPs in screening populations until recently. It is generally accepted that among ever smokers, the presence of chronic obstructive pulmonary disease (COPD) is highly relevant to susceptibility to lung cancer independent of age and smoking exposure. Indeed, studies had shown that the genetic susceptibility to lung cancer among ever smokers included genes that also conferred a risk of COPD. This might explain in part the association between COPD and lung cancer independent of other variables like age and smoking.

Through candidate gene and genome wide association studies (GWAS), several well replicated associations between SNP variants and lung cancer have been reported. More recently, there has been a growing interest in combining genetic data to derive personalized composite risk scores (termed polygenic risk score or PRS). These risk scores are generally based on simple algorithms combining allele or genotype counts according to genotyping results and have been reported to correlate with breast cancer and prostate cancer outcomes. Although none to date have been tested in a prospective cohort study fully representative of high-risk smokers in the community, PRS for lung cancer from case-control studies was reported. PRS have several utilities and might inform clinical practice including targeted screening or preventive strategies for those at higher than average risk. One aspect of this approach is the ability of the PRS to predict the biological behavior or lethality of a cancer and thus the basis for more aggressive (e.g., earlier) surveillance or intervention.

It was suggested that gene-based risk assessment may have utility in the setting of lung cancer screening. This is because the outcomes of screening results from a complex interplay between characteristics of the screening participant (age, comorbid disease and life expectancy) and their lung cancer biology (histology, stage and volume doubling time). The current study examines the utility of a validated 12 SNP panel, in the context of a lung cancer screening trial, to determine its effect on lung cancer biology and screening outcomes. Preliminary results of this study have been reported in abstract form.

### Methods

### Subjects

This was a secondary data analysis of the National Lung Screening Trial (NLST). The recruitment and study design of this trial, involving 53,452 screening participants yielding 2058 histology confirmed lung cancers, has been described elsewhere. In the ACRIN sub-cohort of the NLST, participants from 23 centers agreed to undergo baseline pre-bronchodilator spirometry and, for a subgroup (FIG. 7), blood sampling for biomarker analysis (N=10,054). Demographic data, including history of pre-morbid disease, were collected through an extensive questionnaire. From the total group (all ethnicities), genomic data for Non-Hispanic whites comprising 9,367 high risk smokers from which 380 lung cancers diagnosed during the study follow up (FIG. 7) was analyzed.

### Pulmonary Function Testing

In the NLST-ACRIN cohort, pre-bronchodilator spirometry was measured at baseline screening (TO) in the majority of participants according to a previously published protocol. The spirometry was measured by trained staff using a Spiropro spirometer (eResearchTechnology, GmbH, Germany). The severity of airflow limitation was defined according to the Global Initiative on Chronic Obstructive Lung Disease (GOLD) criteria grades 1-4.

### Lung Cancer Outcomes

Lung cancer cases included all those diagnosed during the trial (N=380), whether screen or non-screen detected (interval), or prevalent (diagnosed at TO or during the first year) or incident lung cancers (diagnosed during subsequent years T1 to T6) or at post-mortem. All lung cancer cases were confirmed on histological sampling according to accepted international classification criteria. Lung function results and mortality outcomes were available for all 380 lung cancer cases (100% of total). Cause of death was ascertained through review of clinical records and death certification.

### Genotyping

Genomic DNA was extracted from buffy coat samples using standard salt-based methods and purified genomic DNA was aliquoted (10 ng/μL concentration) into 384-well plates. DNA concentration and purity were determined by using Nanodrop spectrophotometry. Genotyping entailed using the Sequenom™ system (Sequenom™ Autoflex Mass Spectrometer and Samsung 24 pin nanodispenser) by Agena (Agena BioScience, San Diego, CA, USA) multiplexed into 2 assays (Agena MassARRAY Assay Design 3.0). The Sequenom™ sequences were designed in house by Agena with amplification and separation methods (iPLEX™) as previously described. Replication using this method was undertaken in 2,000 random samples and achieved 99% accuracy for genotyping across the 12 high risk SNPs. Based on prior studies in the literature, the risk genotype/s were pre-specified and assigned as susceptible (Odds >1.0) or protective (Odds <1.0) according to findings (Table 24). A call rate for each SNP of >98% was achieved. 12 risk genotypes (6 susceptible and 6 protective) were identified that were statistically associated with lung cancer and combined according to a previously published algorithm, a lung cancer polygenic risk score (PRS) was derived (Table 10). This was an integer (whole number) ranging from −4 to 6.

### Statistical Analysis

Differences between groups for continuous variables were sought using analysis of variance. Significant differences were further explored as appropriate using Tukey's post hoc method. Differences between groups for categorical variables were sought using chi-squared test (or exact methods where appropriate). General linear modelling (binary distribution, logit link function) was used to estimate the odds of lung cancer death with and without adjustment for clinical risk variables (PLCO_{M2012} model). Mid-P Exact risk and rate differences were used to assess the magnitude and direction of the association. Statistical significance was defined as a two tailed P≤0.05 for comparisons between PRS groups. No adjustment for multiplicity was performed. All planned comparisons are presented in the tables of this paper. All analyses were performed using SAS (V 9.4, SAS Institute Inc, Cary NC) or STATA statistical software.

### Results

Based on the genotype results from 12 independent risk genotypes, a PRS was derived for each participant (N=9191) and sub-grouped them according to low (scores −4 to −1, predominantly protective genotypes), intermediate (neutral score of zero, equal protective and susceptible genotypes) and high (scores 1 to 6, predominantly susceptible genotypes) that encompassed 31% (n=2813), 27% (N=2504), and 42% of subjects (N=3874) respectively (crude tertile). The demographic data for these subgroups are summarised in Table 10. The groups were not significantly different for most of the baseline demographic variables associated with risk of lung cancer specifically, age, gender, smoking status, years smoked, years quit, family history of lung cancer, self-reported history of COPD, BMI, education, and pre-existing comorbidities. Increasing PRS was associated with slightly greater cigarettes per day and pack years but the differences across these risk groups were small (1 cigarette/day and 1 pack year). Despite comparable lung function, higher PRS was associated with slightly greater spirometric-defined COPD (GOLD 1-4) although again the differences were very small (33% vs 36%, P=0.02, Table 10).

TABLE 10
Lung cancer risk factors and demographic variables according to a 12 SNP
polygenic risk score (PRS) severity

| | PRS Groups (absolute score range) | | | | | | |
|---|---|---|---|---|---|---|---|
| | Low Score | | Neutral Score | | High Score | | |
| | (−4 to −1) | | 0 | | (1 to 6) | | P value |
| N = 9,191 (% total) NHW | 2813 | (30.6%) | 2504 | (27.2%) | 3874 | (42.1%) | |
| Demographics | | | | | | | |
| Age-mean (SD) | 61.7 | (5.2) | 61.9 | (5.1) | 61.8 | (5.1) | 0.40 |
| Gender-% Male | 1608 | (57.2%) | 1428 | (57.0%) | 2190 | (56.5%) | 0.86 |
| Smoking Status-Current Smoker | 1374 | (48.8%) | 1161 | (46.4%) | 1882 | (48.6%) | 0.14 |
| Pack Years-mean (SD) | 55.2 | (22.7) | 55.7 | (23.2) | 57.1 | (23.7) | 0.0021 |
| Cigarettes/day-mean (SD) | 27.7 | (10.8) | 28.0 | (10.9) | 28.7 | (11.3) | 0.0002 |
| Years Quit mean (SD) | 3.6 | (5.0) | 3.8 | (5.1) | 3.8 | (5.1) | 0.40 |
| Smoking duration years mean (SD) | 40.3 | (7.1) | 40.4 | (7.5) | 40.3 | (7.6) | 0.93 |
| Family history of Lung Cancer-yes (%) | 671 | (23.9%) | 607 | (24.2%) | 912 | (23.5%) | 0.81 |
| Self-reported Hx of COPD (%)‡ | 561 | (19.9%) | 508 | (20.3%) | 819 | (21.1%) | 0.46 |
| BMI mean (SD) | 27.8 | (5.0) | 27.9 | (5.2) | 27.9 | (5.1) | 0.87 |
| Education Level-N (%) | | | | | | | |
| High School or less | 809 | (28.8%) | 677 | (27.0%) | 1138 | (29.4%) | 0.05 |
| Post High School/Some College- | 995 | (35.4%) | 912 | (36.4%) | 1359 | (35.1%) | |
| College graduate | 508 | (18.1%) | 403 | (16.1%) | 661 | (17.1%) | |
| Postgraduate/Professional | 423 | (15.0%) | 453 | (18.1%) | 617 | (15.9%) | |
| Other/unknown | 78 | (2.8%) | 59 | (2.4%) | 99 | (2.6%) | |
| Lung Function | | | | | | | |
| FEV₁/FVC-mean (SD) | 71.4 | (10.7) | 70.93 | (10.9) | 70.8 | (10.6) | 0.057 |
| FEV₁% predicted-mean (SD) | 81.9 | (19.4) | 81.4 | (20.5) | 80.9 | (20.2) | 0.15 |
| Total COPD GOLD 1-4 | 925 | (32.9%) | 895 | (35.7%) | 1394 | (36.0%) | 0.02 |
| Premorbid conditions (self-report) | | | | | | | |
| COPD | 182 | (6.5%) | 195 | (7.8%) | 285 | (7.4%) | 0.16 |
| Chronic Bronchitis | 327 | (11.6%) | 254 | (10.1%) | 442 | (11.5%) | 0.18 |
| Emphysema | 232 | (8.2%) | 237 | (9.5%) | 378 | (9.8%) | 0.10 |
| Asthma-adult | 181 | (6.4%) | 171 | (6.8%) | 272 | (7.0%) | 0.60 |
| Pneumonia | 772 | (27.4%) | 709 | (28.3%) | 1099 | (28.4%) | 0.67 |
| Heart Disease | 392 | (13.9%) | 312 | (12.5%) | 521 | (13.4%) | 0.28 |
| Hypertension | 1002 | (35.6%) | 890 | (35.5%) | 1374 | (35.5%) | 0.99 |
| Stroke | 91 | (3.2%) | 66 | (2.6%) | 109 | (2.8%) | 0.40 |
| Diabetes | 243 | (8.6%) | 251 | (10.0%) | 342 | (8.8%) | 0.16 |
| Any cancer History | 121 | (4.3%) | 97 | (3.9%) | 163 | (4.2%) | 0.71 |

‡Self-reported History of COPD = “Has a doctor ever told you that you have any of the following conditions.” COPD, Emphysema, Chronic Bronchitis, or Adult Asthma (where prior childhood asthma is excluded). SNP = Single Nucleotide Polymorphism.

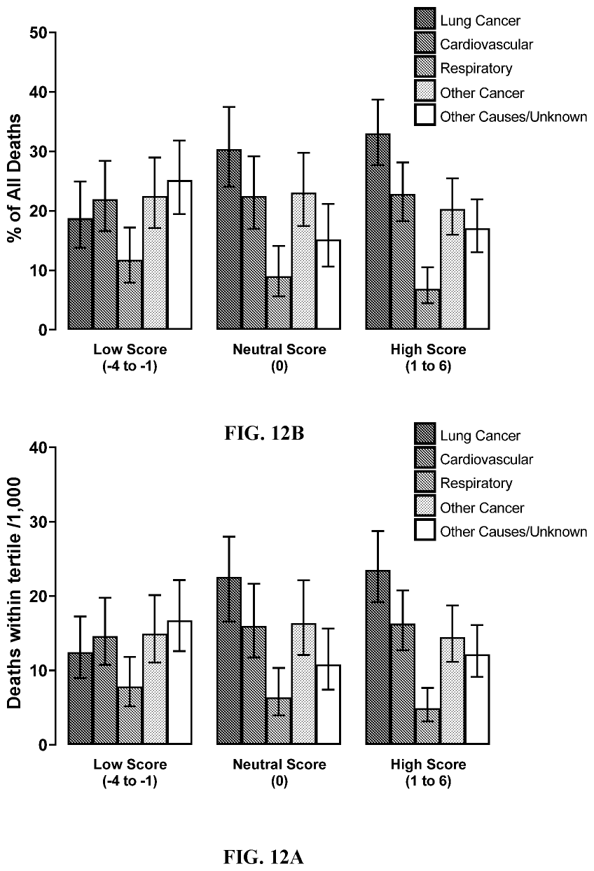

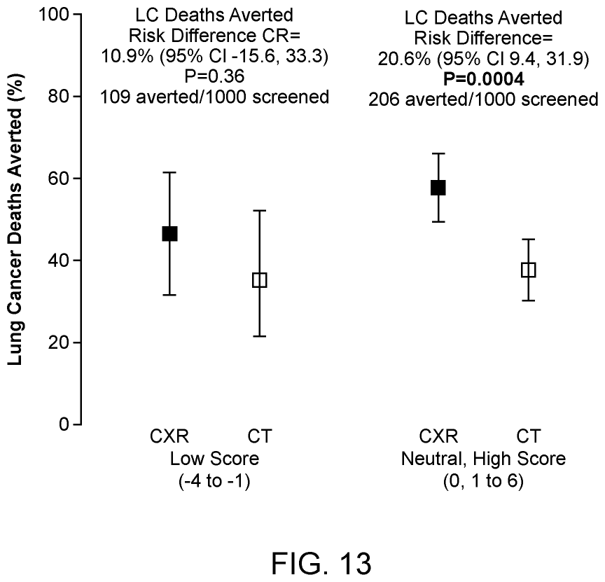

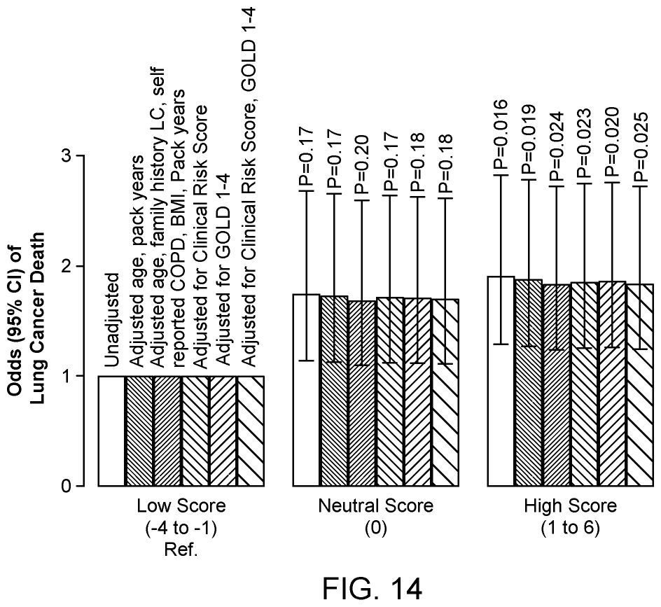

Increasing PRS was associated with a linear increase in lung cancer incidence, absolute lung cancer deaths and lung cancer deaths as a proportion of all deaths (Table 11, FIG. 11, and FIG. 12). Notably, subjects in the intermediate (Neutral) and high risk PRS sub-groups had similar lung cancer mortality (Mortality rate ratios, Table 11), which were 1.7 and 1.9 fold greater respectively (P=0.01 and P=0.0009) than those in the low PRS group (Table 11 and FIG. 11). These findings were evident despite very similar lung function, histology, stage, detection method, surgical rates and diagnostic screening interval (Table 12). When cause-specific deaths according to increasing PRS were compared, only lung cancer mortality demonstrated a positive relationship (Table 11 and FIG. 12) with a compensatory drop in “other” causes of death. After considerable adjustment for various clinical variables (FIG. 14), it was found that the lung cancer mortality remained significantly greater in the high versus low PRS groups. After randomisation, screening participants in the low PRS group (predominantly protective genotypes) had an 11% relative reduction in lung cancer deaths favouring CT (P=0.36) while the other sub-group (intermediate and high) was 22%, (P=0.0004). This was in conjunction with comparable surgical rates favouring those randomised to CT screening over CXR (15%, P=0.21 and 14%, P=0.019 respectively) and comparable lung cancer characteristics (Table 12). Importantly, the risk difference for lung cancer deaths averted after randomisation favoured those in the combined intermediate and high sub-group (58% vs 37%, P=0.0004, FIG. 13), where overall mortality was no different (rate ratio=1.09, P=0.62, Table 11).

It was previously described that a gene-based risk algorithm that combines clinical variables with PRS to derive a composite individualised risk for developing and/or dying of lung cancer (Table 13). The predefined scoring algorithm for the clinical variables are: age ≥65 years old (+4), history of COPD (+4), Family history (1^{st} degree) of lung cancer (+3), abnormal BMI (BMI ≤20=+4 and BMI ≥25=−1), smoking pack years (pack years ≤39=−2 and pack years ≥60=+2). As described above, the pre-defined PRS algorithm combines the total susceptible risk genotypes (+1 for each present out of 6 possible risk genotypes) and total protective risk genotypes (−1 for each present out of 6 possible risk genotypes). Using area-under-the-curve (AUC) receiver operating characteristics (ROC) analyses to derive a C-statistic, it was found that the risk of dying of lung cancer according to the PRS alone was AUC-0.568 (95% Confidence Interval (CI)=0.528-0.608, P=0.0009) and according to PRS and clinical score AUC=0.661 (95% CI=0.623-0.698, P<0.0001). Using Odds Ratio Estimates to model dying of lung cancer, it was found that each clinical score of 1 added 0.15 increase to the Odds of lung cancer death and each PRS score of 1 added 0.17 increase indicating comparable weighting in the model. Using the same analysis for risk of developing lung cancer it was found that for the clinical score was AUC=0.647, 95% CI 0.620-0.674, P<0.0001, while adding the PRS score increased the AUC (AUC=0.667, 95% CI 0.640-0.693, P=0.018). When this was limited to those in the CT arm, the clinical only AUC (0.640, 95% CI 0.602-0.678) increased when adding the PRS (0.668, 95% CI 0.602-0.705, P=0.011). Using the PLCO_{M2012} model for developing lung cancer in this cohort, an AUC=0.677 (95% CI 0.651-0.704, P<0.001) was obtained, significantly different from the AUC of a model with no discriminability (i.e., an AUC of 0.500).

TABLE 11
Overall mortality and reduction in lung cancer deaths after screening according to polygenic
risk score (PRS) groups (Low, Neutral, or High)

| | PRS Groups (absolute score range) | | | | | | |
|---|---|---|---|---|---|---|---|
| | Low score | | Neutral Score | | High score | | P |
| | (−4 to −1) | | 0 | | (1 to 6) | | value^{β} |
| N = 9, 191 NHW (% of total cohort) | 2813 | (30.6%) | 2504 | (27.2%) | 3874 | (42.1%) | |
| Lung Cancer | | | | | | | |
| Total lung cancer^{‡} (% total, N = 380) | 78 | (20.5%) | 102 | (26.8%) | 200 | (52.6%) | <0.0001 |
| Incidence/per 1000 ^{Φ}(95% CI) | 27.7 | (22.3, 34.5) | 40.7 | (33.7, 49.2) | 51.6 | (45.1, 59.1) | |
| Overall Mortality (prevalence) | | | | | | | |
| Total Deaths (%, per 100) | 187 | (6.6%) | 178 | (7.1%) | 276 | (7.1%) | 0.72 |
| | 187/641 | | 178/641 | | 276/641 | | |
| % and 95% CI all deaths (N = 641) | 29.2%, | (25.8, 32.9) | 27.8% | (24.4, 31.4) | 43.1% | (39.3, 46.9) | |
| Lung Cancer (LC) Deaths (%, per 100) | 35/187 | (18.7%) | 54/178 | (30.3%) | 91/276 | (33.0%) | 0.0027 |
| % all deaths (N = 641) | 5.5% | (4.0, 7.5) | 8.4% | (6.5, 10.8) | 14.2% | (11.7, 17.1) | |
| % all deaths by tertile | 1.2% | (0.90, 1.73) | 2.2% | (1.7, 2.8) | 2.3% | (1.9, 2.9) | |
| Cardiovascular(CVD) Deaths | 41 | (21.9%) | 40 | (22.5%) | 63 | (22.8%) | 0.97 |
| % all deaths (N = 641) | 6.4% | (4.8, 8.6) | 6.2% | (0.2, 1.6) | 9.8% | (7.8, 12.4) | |
| % all deaths by tertile | 1.5% | (1.1, 2.0) | 1.6% | (1.2, 2.2) | 1.6% | (1.3, 2.1) | |
| Respiratory Deaths | 22 | (11.8%) | 16 | (9.0%) | 19 | (6.9%) | 0.19 |
| % all deaths (N = 641) | 3.4% | (2.3, 5.1) | 2.5% | (1.5, 4.0) | 3.0% | (1.9, 4.6) | |
| % all deaths by tertile | 0.78% | (0.52, 1.18) | 0.64% | (0.39, 1.04) | 0.49% | (0.31, 0.76) | |
| Other Cancer Deaths | 42 | (22.5%) | 41 | (23.0%) | 56 | (20.3%) | 0.75 |
| % all deaths (N = 641) | 6.6% | (4.9, 8.7) | 6.4% | (4.8, 8.6) | 8.7% | (6.8, 11.2) | |
| % all deaths by tertile | 1.5% | (1.1, 2.0) | 1.6% | (1.2, 2.2) | 1.4% | (1.1, 1.9) | |
| Other Deaths or COD unknown | 47 | (25.1%) | 27 | (15.2%) | 47 | (17.0%) | 0.030 |
| % all deaths (N = 641) | 7.3% | (5.6, 9.6) | 4.2% | (2.9, 6.1) | 7.3% | (5.6, 9.6) | |
| % all deaths by tertile | 1.7% | (1.3, 2.2) | 1.1% | (0.7, 1.6) | 1.2% | (0.9, 1.6) | |

| | | | | | | |
|---|---|---|---|---|---|---|
| Lung Cancer Mortality-rate | | | | | | |
| ratios‡ | | | | | | |
| Low vs Neutral | ref | 1.73 | (1.14-2.64), | 1.89 | (1.28-2.78), | — |
| (95% CI), P value | | P | = 0.010 | P | = 0.0009 | |

| | | | | | |
|---|---|---|---|---|---|
| Neutral vs High | — | ref | 1.09 | (0.78-1.52), | — |
| (95% CI), P value | | | P | = 0.62 | |

After Randomization (N = 367 which excludes lung cancers diagnosed at post-mortem)

| | Lung cancer Screening Outcomes | | | | | | |
|---|---|---|---|---|---|---|---|
| | | | Neutral-CXR | | Neutral + High- | | |
| | Low-CXR vs CT | | vs CT | | CXR vs CT | | |
| Surgery rates % (95% CI) favoring | 17/39 v 20/34 | | [symbol] /41 v | | 3/92 v | | — |
| CT over CXR across PRS groups | 44% | (29, 59) v | 29/56 | [symbol] 30, 59) v | 67/105 | (37, 57) v | |
| | 59% | (42, 74) | 52% | (39, 64) | 64% | (54, 72) | |
| Risk difference-% (95% CI) | −15% | (−38, 7.5) | −8% | (−28, 12) | −17% | (−32, −3.3) | — |
| P value | P | = 0.21 | P | = 0.45 | P | = 0.017 | |

| | | | Neutral + High-CXR | | |
|---|---|---|---|---|---|
| | Low-CXR vs CT | | Vs CT | | |
| Outcomes Low vs Neutral + High | | | | | |
| Surgery rates % (95% CI) favoring | 17/39 v 20/34 | | 61/133 v 96/161 | | — |
| CT over CXR for PRS sub-group | 44% | (29, 59) v) | 46% | (38, 54) v | |
| | 59% | (42, 74 | 60% | (52, 67) | |
| Risk difference-% (95% CI) | −15% | (−38, 7.5) | −14% | (−25, −2.41) | — |
| P value | P | = 0.21 | P | = 0.019 | |
| Lung cancer deaths | | | | | |
| Deaths averted favoring CT over | 18/39 v 12/34 | | 77/133 v 60/161 | | — |
| CXR^{‡‡}-% (95% CI) | 46% | (32, 61) v | 58% | (49, 66) v | |
| | 35% | (21, 52) | 37% | (30, 45) | |
| Risk difference-% (95% CI) | +11.0 | (−12, 33) | +21% | (9.4, 32) | — |
| P value | P | = 0.36 | P | = 0.0004 | |
| Odds ratio (OR) of lung cancer death | OR = 1.57 | (0.61, 4.04) | OR = 2.32 | (1.15, 3.70) | — |
| in CXR vs CT (95% CI) | P | = 0.35 | P | = 0.0005 | |
| P value | | | | | |

^{‡}Includes lung cancer deaths identified at post-mortem (N = 180), ^{‡‡}Only lung cancers diagnosed during screening and follow-up (N = 367). ^{β}P value according to PRS group. (95% CI) = 95% confidence Interval. [symbol]
indicates data missing or illegible when filed

TABLE 12
Lung cancer (LC) characteristics according to the lung cancer polygenic risk score (PRS)
score (Low, Neutral, or High)

| | PRS Groups (absolute score range) | | | | | | |
|---|---|---|---|---|---|---|---|
| | Low Score | | Neutral Score | | High Score | | |
| | (−4 to −1) | | 0 | | (1 to 6) | | P value |
| N = 9,191 NHW (% of total cohort) | 2813 | (30.6%) | 2504 | (27.2%) | 3874 | (42.2%) | Total |
| Lung Cancers-N‡ (per 1000) | 78 | (27.7) | 102 | (40.7) | 200 | (51.6) | <0.0001 |
| Demographics | | | | | | | |
| Age-mean (SD) | 64.2 | (5.0) | 63.3 | (5.2) | 64.2 | (5.5) | 0.37 |
| Male Gender (% Male) | 46 | (59.0%) | 63 | (61.8%) | 104 | (52.0%) | 0.23 |
| Pack Years-mean (SD) | 63.6 | (29.1) | 62.5 | (24.0) | 64.1 | (25.9) | 0.88 |
| Smoking Status (% Current) | 49 | (62.8%) | 58 | (56.9%) | 107 | (53.5%) | 0.37 |
| Self-reported Comorbidity | | | | | | | |
| COPD | 20 | (25.6%) | 36 | (35.3%) | 64 | (32.0%) | 0.38 |
| Pneumonia | 23 | (29.5%) | 34 | (33.3%) | 67 | (33.5%) | 0.80 |
| Heart Dx | 8 | (10.3%) | 20 | (19.6%) | 31 | (15.5%) | 0.23 |
| Diabetes | 5 | (6.4%) | 11 | (10.8%) | 18 | (9.0%) | 0.59 |
| Hypertension | 33 | (42.3%) | 37 | (36.3%) | 74 | (37.0%) | 0.66 |
| Stroke | 1 | (1.3%) | 6 | (5.9%) | 6 | (3.0%) | 0.38 |
| Lung Function | | | | | | | |
| FEV₁/FVC-mean (SD) | 66.57 | (10.76) | 66.10 | (12.0) | 66.39 | (11.8) | 0.96 |
| FEV₁% predicted-mean (SD) | 71.57 | (20.4) | 74.99 | (20.8) | 74.28 | (23.0) | 0.55 |
| GOLD 1-2 | 29 | (37.2%) | 36 | (35.3%) | 74 | (37.0%) | 0.95 |
| GOLD 3-4 | 14 | (18.0%) | 14 | (13.7%) | 28 | (14.0%) | 0.67 |
| Lung Cancer (LC) Characteristics | | | | | | | |
| Lung Cancer Death N (% of LC)‡ | 35/78 | (44.9%) | 54/102 | (52.9%) | 91/200 | (45.5%) | 0.43 |
| Lung Cancer Death N (% of total grp) | 35/2813 | (1.2%) | 54/2504 | (2.2%) | 91/3874 | (2.3%) | 0.0040 |
| Lung Cancer Death during study | 30/73 | (41.1%) | 49/97 | (50.5%) | 88/197 | (44.7%) | 0.45 |
| (% of LC)† | | | | | | | |
| Lung cancer Histology | | | | | | | |
| Small Cell | 12 | (15.4%) | 8 | (7.8%) | 28 | (14.0%) | 0.46 |
| Squamous Cell | 18 | (23.1%) | 26 | (25.5%) | 42 | (21.0%) | |
| Adenocarcinoma/BAC^{Φ} | 29 | (37.2%) | 46 | (45.1%) | 90 | (45.0%) | |
| Non-small/Large Cell | 14 | (18.0%) | 17 | (16.7%) | 36 | (18.0%) | |
| Other/unknown | 5 | (6.4%) | 5 | (4.9%) | 4 | (2.0%) | |
| Lung cancer Stage at Diagnosis N (%) | | | | | | | |
| Stage I-II | 38 | (48.7%) | 42 | (41.2%) | 100 | (50.0%) | 0.35 |
| Stage III-IV | 34 | (43.6%) | 53 | (52.0%) | 93 | (46.5%) | |
| Occult carcinoma/unknown | 6 | (7.7%) | 7 | (6.9%) | 7 | (3.5%) | |
| Surgery-Yes | 37 | (47.4%) | 47 | (46.1%) | 110 | (55.0%) | 0.26 |
| Interval lung Cancer diagnosed | | | | | | | |
| Screening (T0-T2) | 47 | (60%) | 59 | (58%) | 123 | (61%) | 0.83 |
| Follow up (T3-T7) | 31 | (40%) | 43 | (42%) | 77 | (39%) | |
| Cancer detection | | | | | | | |
| Screen-detected | 40 | (51.3%) | 45 | (44.1%) | 104 | (52.0%) | 0.64 |
| Interval/Missed | 7 | (9.0%) | 14 | (13.7%) | 19 | (9.5%) | |
| Follow up | 31 | (39.7%) | 33 | (32.3%) | 77 | (38.5%) | |

‡Total lung cancers including those diagnosed at post-mortem (N = 13), †Excludes LC diagnosed at post-mortem. ^{Φ}BAC = Bronchioloalveolar Carcinomas

TABLE 13
Algorithm and referencing for the polygenic risk score (PRS)
and clinical score for lung cancer

| | Clinical score | | |
|---|---|---|---|
| | Criteria | Score | |
| Age | ≥65 years old | +4 | |
| Past history of COPD | Yes | +4 | |
| Family history of Lung Cancer | Yes | +3 | |
| Body Mass Index | ≤20 | +4 | |
| | 20-24 | | |
| | ≥25 | −1 | |
| Smoking history | 30-39 pack years | −2 | |
| | 40-59 pack years | | |
| | ≥60 pack years | +2 | |
| | Polygenic risk score (PRS) | | |
| | Gene (rs #) | Score | |
| 1 | CHRNA (rs16969968) | AA | = +1 |
| 2 | BAT3 (rs1052486) | CC | = +1 |
| 3 | TERT (rs402710) | CC | = +1 |
| 4 | FasL (rs763110) | TT | = −1 |
| 5 | Rev1 (rs3087386) | CC | = −1 |
| 6 | MMP12 (rs645419) | GG | = −1 |
| 7 | ITGA11 (rs2306022) | TT/TC | = −1 |
| 8 | IL-6 (rs1800797) | GG | = +1 |
| 9 | IREB (rs2656069) | CC/CT | = −1 |
| 10 | CYP2A6 (rs7937) | TT | = +1 |
| 11 | CRP (rs2808630) | CC | = −1 |
| 12 | AGER (rs2070600) | TT/TC | = +1 |

### Discussion

In this clinical validity study using a subgroup of the NLST, a lung cancer polygenic risk score (PRS), derived from 12 independent risk genotypes, correlate with lung cancer mortality. The PRS is based on germ-line mutations and is independent of significant lung cancer risk factors notably, age, smoking, family history, education, BMI, lung function and pre-existing comorbidity. While it was expected that there to be more cancers developing in those with the higher PRS scores, this study shows that elevated PRS was also significantly and linearly associated with greater lung cancer deaths. This could not be explained by variation in lung cancer stage, histology, detection methods, screening interval or surgical rates. Although there was a weak association with smoking pack years across the PRS groups, due primarily to smoking one extra cigarette per day, this would not explain these findings. Similarly, there was marginally more airflow limitation in those with an elevated PRS but neither of these, nor any other clinical variables examined after extensive adjustment, could account for the clear differences in lung cancer mortality. The differences in lung cancer mortality were not related to differences in lung cancer risk factors or comorbidity. The greater reduction in lung cancer mortality observed in those randomised to CT in the elevated PRS sub-group could not be explained by differences in surgical rates.

There has been considerable interest in assessing high risk smokers eligible for lung cancer screening using clinical risk models. The most validated of these risk models is the PLCO_{M2012} which combines a number of clinical risk variables to derive a 6-year risk for lung cancer. However, it can be shown that this risk score also predicts the likelihood of having airflow limitation and COPD. This is important because pre-existing COPD is associated with greater lung cancer risk, more aggressive lung cancer histological subtypes, less surgery and reduced benefit of lung cancer screening. This means those at greatest risk of lung cancer may not achieve the greatest benefits from screening with an attenuation of this risk-benefit relationship at the highest end of the risk spectrum. As the benefits of lung cancer screening results from a complex interplay between a number of variables, it might be helpful to identify a biomarker of lung cancer lethality as part of the general risk assessment of screening participants. This is because it is recognised there exist considerable biological variability between lung cancers and that this variability will affect outcomes following screening. Screening participants with a low PRS (protective risk genotypes) may have more indolent lung cancers that contribute less to a mortality reduction with screening. Conversely, those with a higher PRS are more prone to aggressive lung cancer (intermediate and short doubling times) where CT screening is more effective. As such, a biomarker of lung cancer biology can be combined with clinical risk variables to better determine which screening participants gain the greatest benefit from screening.

The utility of adding genetic data to clinical risk scores of cancer has been gaining support in the clinical setting of optimising screening for prostate and breast cancer. Investigators have previously shown that SNP based data adds small but clinically meaningful contribution to risk stratifying high risk populations. In this setting, this example has that PRS, based on previously validated SNPs, adds to the risk assessment for lung cancer in a lung cancer screening cohort where lung cancer was diagnosed prospectively. How the composite score may contribute to optimising lung cancer screening is the subject of another study. However, based on the current analysis it appears that the PRS is not only associated with a modestly greater risk of developing lung cancer (improves the AUC when added to the clinical score), it is also associated with greater mortality from lung cancer. This latter finding persisted after extensive adjustment for various clinical variables and was also independent of known lung cancer variables such as stage and histology.

In short, this example illustrates a polygenic risk score can be derived for lung cancer that correlates with lung cancer lethality in a prospective clinical validity study. It can be suggested that this score may contribute to assessing lung cancer screening participants with the aim to better identify screening participants who might benefit most. The latter is the subject of a clinical utility study. This gene-based approach to selecting screening participants may have cost-effective benefits by reducing the number needed to screen (NNS) to prevent one lung cancer. This may have considerable importance in the younger lighter smokers recently recommended to be included in lung cancer screening. This approach deviates from existing approaches which recommends targeting those at greatest risk (risk-based selection) to lung cancer screening. It can be suggested that an outcomes-based approach to lung cancer screening may be of greater utility and that germ-line mutations represent a very stable, cheap and acceptable bioassay to incorporate into the optimisation of screening programmes.

In this clinical validity study from a subgroup of the NLST, it was reported that a lung cancer polygenic risk score (PRS), derived from 12 risk genotypes, appears to correlate with lung cancer mortality. The PRS is based on germ-line mutations and is independent of significant lung cancer risk factors notably, age, smoking, family history, education, BMI, lung function and pre-existing comorbidity. While it was expected that there to be more cancers developing in those with the higher scores, it was found that a high PRS was also significantly and linearly associated with greater lung cancer deaths. This could not be explained by variation in lung cancer stage, histology, detection methods, screening interval or surgical rates. Although there was a weak association with smoking pack years across the PRS groups, due primarily to smoking one extra cigarette per day, this would not explain the findings. Similarly, there was marginally more airflow limitation in those with an elevated PRS but neither of these would account for the clear differences in lung cancer mortality. The differences in lung cancer mortality were not related to differences in lung cancer risk factors or comorbidity. The smaller reduction in lung cancer mortality observed in those randomized to CT in the high PRS group could not be explained by differences in surgical rates. It was concluded that the lung cancer PRS provides an independent biomarker of lung cancer lethality.

There has been considerable interest in assessing high risk smokers eligible for lung cancer screening using clinical risk models. The most validated of these risk models is the PLCO_{M2012} which combines a number of clinical risk variables to derive a 6-year risk for lung cancer. However, it was shown that this risk score also predicts the likelihood of having airflow limitation. This is important because pre-existing COPD is associated with greater lung cancer risk, more aggressive lung cancer histological subtypes, less surgery and reduced benefit of lung cancer screening. This means those at greatest risk of lung cancer may not achieve the greatest benefits from screening with an attenuation of this risk-benefit relationship. As the benefits of lung cancer screening results from a complex interplay between a number of variables, it might be helpful to identify a biomarker of lung cancer lethality as part of the general risk assessment of screening participants. This is because it is recognized there exist considerable biological variability between lung cancers and that this variability will affect outcomes following screening. Lung cancers that have intermediate volume doubling times may be those most amenable to CT screening. The very indolent lung cancers (long doubling time), best exemplified by ground glass opacities (GGOs), are easily identified and removed but may not kill the smoker. In contrast, very aggressive cancers may metastasize early and result in death despite detection in an early stage by CT-based screening. Accordingly, it was suggested that a biomarker of lung cancer biology could be combined with clinical risk variables to better determine which screening participants gain the greatest benefit from screening. This is the subject of further analysis.

In summary, in the current study a polygenic risk score was derived for lung cancer that appears to correlate with lung cancer lethality in a prospective clinical validity study. It was suggested that this score may contribute to assessing lung cancer screening participants with the aim to better identify lung cancer screening participants who might benefit most. The latter is the subject of a clinical utility study. This gene-based approach to selecting screening participants may have cost-effective benefits by reducing the number needed to screen (NNS) to prevent one lung cancer. This approach deviates from existing approaches which recommends targeting those at greatest risk (risk-based selection) to lung cancer screening. It was suggested that an outcomes-based approach to lung cancer screening may be of greater utility and that germ-line mutations represent a very stable, cheap and acceptable bioassay to incorporate into screening programs.

While the foregoing disclosure has been described in some detail for purposes of clarity and understanding, it will be clear to one skilled in the art from a reading of this disclosure that various changes in form and detail can be made without departing from the true scope of the disclosure. For example, all the techniques and apparatus described above can be used in various combinations. All publications, patents, patent applications, and/or other documents cited in this application are incorporated by reference in their entirety for all purposes to the same extent as if each individual publication, patent, patent application, and/or other document were individually and separately indicated to be incorporated by reference for all purposes.

### Example 4. Gene-Based Risk Score Individualizes Benefit and Increases Screening Efficiency in the National Lung Screening Trial: A Post-Hoc Clinical Utility Study

Current eligibility criteria for lung cancer screening are derived from randomized controlled trials and primarily based on age and smoking history. While there is growing interest in improving screening efficiency, by using lung cancer risk models utilizing several clinical risk variables, recent focus has turned to the clinical utility of using gene-based approaches for cancer screening.

This was a post-hoc analysis of 9.191 Non-Hispanic white subjects from the National Lung Screening Trial (NLST), a sub-study of high risk smokers randomized to annual compute tomography (CT) or chest x-ray and followed for an average of 6.1 years. This study compared the screening efficiency of a recently validated gene-based risk score with a well validated clinical score (PLCO_{M2012}).

In this clinical utility study, the relative reduction in lung cancer mortality with CT-based screening varied according to no stratification (25%), or after stratification across PLCO_{M2012} quintiles 2-4 (27%) and gene-based risk quintiles 2-4 (43%, risk ratio=0.57.95% CI=0.38-0.84, P=0.0044). The corresponding absolute benefits according to lung cancer deaths averted with CT were 5.1/1000, 5.3/1000 and 10.3/1000 persons screened. The number needed to screen to avert one lung cancer death was 196, 188 and 97 respectively.

Conclusion: In this biomarker-based clinical utility study, based on a sub-cohort from the NLST, a gene-based approach to stratifying and targeting lung cancer screening achieved the greatest efficiency in reducing lung cancer mortality was found. In smokers of intermediate risk (quintiles 2-4), combining genetic and clinical risk identified those who benefit the most from screening.

### INTRODUCTION

Lung cancer results in the main from the combined effect of genetic susceptibility and long-term smoking exposure. Epidemiological studies have identified a number of clinical risk variables underlying lung cancer susceptibility including age and smoking history and these have been combined in risk models to derive a composite risk for future lung cancer. These risk models have been validated in independent cohorts to a variable degree and performance characteristics. The best known of these clinical risk tools (PLCO_{M2012}) comes from the Prostate. Lung. Colorectal and Ovarian (PLCO) cancer screening study and subsequently validated in the National Lung Screening trail (NLST). Based on a 20% relative reduction in lung cancer deaths favoring the computed tomography arm of the NLST, CT-based screening for lung cancer is widely recommended in the USA. The positive findings of this study were recently replicated in a European CT-based screening trial where relative reductions in lung cancer death were 33% in women and 24% in men. In both of these studies, the eligible population of ever smokers were defined according to age and smoking criteria (risk-based approach). These studies provide the basis of recently updated selection criteria for lung cancer screening, although individualized risk across eligible screening populations has been shown to vary widely.

One limitation of a risk-based approach is the assumption that increasing risk is associated with increasing benefits from screening. When studies adjust for comorbid disease, competing deaths and life expectancy, they conclude that smokers at greatest risk of lung cancer have an attenuated benefit from screening. These findings suggest the benefits of screening are greatest in those at intermediate risk, specifically those in the 20%-80% risk (optimized screening in quintiles 2-4). A second limitation of a risk based-approach stems from the assumption that age and pack years are the best markers of lung cancer risk in older heavy smokers when a study in the NLST showed airflow limitation has an even greater effect on risk of developing lung cancer. This is a problem because increasing PLCO_{M2012} risk is associated with greater prevalence of chronic obstructive pulmonary disease (COPD) which itself is linked to cardiorespiratory disease and premature death. It has been shown that smokers in the NLST with COPD have only half the benefit from lung cancer screening than those with normal lung function despite being at 2-3 fold greater risk. While many reasons exist for this poorer outcome, it has been found that airflow limitation (COPD) in the NLST was associated with greater comorbid disease, greater non-lung cancer mortality, more aggressive lung cancer and reduced surgical rates compared to those with normal lung function. Together, these observations might explain why those at intermediate risk (quintiles 2-4) represent those who gain the most from screening when applying an optimized or outcomes-based approach.

There has been a growing interest in the utility of combining genetic data to derive polygenic risk scores (PRS) to better define cancer susceptibility. These scores can be derived from well validated genetic variants, primarily single nucleotide polymorphisms (SNPs), and used to better define at risk populations for targeted interventions such as screening. These polygenic risk scores are combined with clinical data to generate a composite score which generally predicts susceptibility to the disease of interest. It is important that not only should these gene-based risk scores correctly predict meaningful outcomes (clinical validity), they should also help address important decisions about screening (clinical utility), including how to best to minimize the number needed to screen to prevent one lung cancer death (screening efficiency). In an ideal situation, the clinical validity and utility of biomarker-based tools should be confirmed in a prospective study in a cohort representative of the “at risk” population and in the setting of the intervention in question. In the NLST, stored bio-specimens provide a unique opportunity to validate a gene-based risk of lung cancer and it's clinical utility in the context of lung cancer screening outcomes. Results of this study have been reported herein.

### Methods

### Subjects

This is a retrospective biomarker-based validation study from the National Lung Screening Trial (NLST), where baseline buffy-coat samples for germ-line mutation analysis were collected in a subgroup. The recruitment and study design of the full NLST, involving 53,452 screening participants yielding 2058 histology confirmed lung cancers, has been described elsewhere. In the ACRIN sub-cohort of the NLST, participants from 23 centers consented to undergo baseline pre-bronchodilator spirometry and, for a subgroup (FIG. 7), buffy coat sampling for genomic analysis (N=10,054). Demographic data, including history of pre-morbid disease, were collected through an extensive questionnaire. From the total group (all ethnicities), genomic data for Non-Hispanic whites only comprising 9,367 high risk smokers from which 380 lung cancers were diagnosed during the study follow up (FIG. 7).

### Lung Cancer Outcomes

Lung cancer cases included all those diagnosed during the trial (N=380), whether screen or non-screen detected (interval), or prevalent (diagnosed at TO or during the first year) or incident lung cancers (diagnosed during subsequent years T1 to T6) or at post-mortem. All lung cancer cases were confirmed on histological sampling according to accepted international classification criteria. Lung function results and mortality outcomes were available for all 380 lung cancer cases. Cause of death was ascertained through review of clinical records and death certification.

### Statistical Analysis

Data were analysed between groups using analysis of variance (ANOVA) for normally distributed variables or chi-squared tests for categorical variables. With post hoc comparison of significant differences between group differences maintained at the 5% significance level with false discovery rate protected p values. Logistic regression was used to estimate the area under the receiver operating characteristics (ROC) curves (as the c-statistic) for models predicting risk of developing or dying from lung cancer from clinical or gene-based models. Analyses were performed using SAS (V 9.4, SAS Institute Inc, Cary NC) Odds ratios, risk ratios and rate ratios for calculated and compared using openepi.com (www.openepi.com, accessed 1^{st} August 2021). Statistical significance was defined as a two tailed P<0.05. As well as comparing the predictive utility of the gene-based test with the PLCO_{M2012} model, the clinical utility of this test applying an outcomes-based approach was also assessed, which minimizes the number needed to screen to prevent one lung cancer death.

### Results

### Clinical Variables According to the Polygenic Risk Score (PRS)

Based on the genotype results from 12 independent risk genotypes, a PRS was derived for each participant (N=9191) and sub-grouped them according to low (scores −4 to −1, predominantly protective genotypes), intermediate (neutral score of zero, equal protective and susceptible genotypes) and high (scores 1 to 6, predominantly susceptible genotypes) that encompassed 31% (n=2813), 27% (N=2504) and 42% of subjects (N=3874) respectively (crude tertiles). The demographic data for these subgroups are summarized in Table 14 and show that the PRS groups were not significantly different for most of the baseline demographic variables associated with risk of lung cancer. A high PRS was associated with slightly greater cigarettes per day and pack years but the differences across these risk groups were small (1 cigarette/day and 1 pack year). Despite comparable lung function, higher PRS (Neutral and High) was associated with slightly greater spirometric-defined COPD (GOLD 1-4) although again the differences were very small (33% vs 36%, P=0.02) (Table 14). A low PRS was associated with a reduced likelihood of lung cancer death (Table 14) despite comparable histology, stage and surgical rates.

TABLE 14
Lung cancer risk factors and demographic variables according to a 12 SNP polygenic risk
score (PRS, genetic data alone) in crude tertiles

| | PRS Groups from a 12 SNP panel (absolute score range) | | | | | | |
|---|---|---|---|---|---|---|---|
| | Low Score | | Neutral Score | | High Score | | |
| | (−4 to −1) | | (0) | | (1 to 6) | | P value |
| N = 9,191 (% total) NHW | 2813 | (30.6%) | 2504 | (27.2%) | 3874 | (42.1%) | |
| Demographics | | | | | | | |
| Age-mean (SD) | 61.7 | (5.2) | 61.9 | (5.1) | 61.8 | (5.1) | 0.40 |
| Gender-% Male | 1608 | (57.2%) | 1428 | (57.0%) | 2190 | (56.5%) | 0.86 |
| Smoking Status-Current Smoker | 1374 | (48.8%) | 1161 | (46.4%) | 1882 | (48.6%) | 0.14 |
| Pack Years-mean (SD) | 55.2 | (22.7) | 55.7 | (23.2) | 57.1 | (23.7) | 0.0021 |
| Cigarettes/day-mean (SD) | 27.7 | (10.8) | 28.0 | (10.9) | 28.7 | (11.3) | 0.0002 |
| Years Quit mean (SD) | 3.6 | (5.0) | 3.8 | (5.1) | 3.8 | (5.1) | 0.40 |
| Smoking duration years mean (SD) | 40.3 | (7.1) | 40.4 | (7.5) | 40.3 | (7.6) | 0.93 |
| Family history of Lung Cancer-yes (%) | 671 | (23.9%) | 607 | (24.2%) | 912 | (23.5%) | 0.81 |
| Self-reported Hx of COPD (%)‡ | 561 | (19.9%) | 508 | (20.3%) | 819 | (21.1%) | 0.46 |
| BMI mean (SD) | 27.8 | (5.0) | 27.9 | (5.2) | 27.9 | (5.1) | 0.87 |
| Education Level-N (%) | | | | | | | |
| High School or less | 809 | (28.8%) | 677 | (27.0%) | 1138 | (29.4%) | 0.05 |
| Post High School/Some College- | 995 | (35.4%) | 912 | (36.4%) | 1359 | (35.1%) | |
| College graduate | 508 | (18.1%) | 403 | (16.1%) | 661 | (17.1%) | |
| Postgraduate/Professional | 423 | (15.0%) | 453 | (18.1%) | 617 | (15.9%) | |
| Other/unknown | 78 | (2.8%) | 59 | (2.4%) | 99 | (2.6%) | |
| Lung Function | | | | | | | |
| FEV₁/FVC-mean (SD) | 71.4 | (10.7) | 70.9 | 3(10.9) | 70.8 | (10.6) | 0.057 |
| FEV₁% predicted-mean (SD) | 81.9 | (19.4) | 81.4 | (20.5) | 80.9 | (20.2) | 0.15 |
| Total COPD GOLD 1-4 | 925 | (32.9%) | 895 | (35.7%) | 1394 | (36.0%) | 0.02 |
| Pre morbid conditions (self-report) | | | | | | | |
| COPD | 182 | (6.5%) | 195 | (7.8%) | 285 | (7.4%) | 0.16 |
| Chronic Bronchitis | 327 | (11.6%) | 254 | (10.1%) | 442 | (11.5%) | 0.18 |
| Emphysema | 232 | (8.2%) | 237 | (9.5%) | 378 | (9.8%) | 0.10 |
| Asthma-adult | 181 | (6.4%) | 171 | (6.8%) | 272 | (7.0%) | 0.60 |
| Pneumonia | 772 | (27.4%) | 709 | (28.3%) | 1099 | (28.4%) | 0.67 |
| Heart Disease | 392 | (13.9%) | 312 | (12.5%) | 521 | (13.4%) | 0.28 |
| Hypertension | 1002 | (35.6%) | 890 | (35.5%) | 1374 | (35.5%) | 0.99 |
| Stroke | 91 | (3.2%) | 66 | (2.6%) | 109 | (2.8%) | 0.40 |
| Diabetes | 243 | (8.6%) | 251 | (10.0%) | 342 | (8.8%) | 0.16 |
| Any cancer History | 121 | (4.3%) | 97 | (3.9%) | 163 | (4.2%) | 0.71 |
| Lung Cancer Mortality-rate ratios | | | | | | | |

| | | | | | | |
|---|---|---|---|---|---|---|
| Low vs Neutral PRS | ref | 1.73 | (1.14-2.64) | 1.89 | (1.28-2.78) | — |
| (95% Confidence Interval) | | | | | | |
| P value | | P | = 0.010 | P | = 0.0009 | |

| | | | | |
|---|---|---|---|---|
| Neutral vs High PRS | — | ref | 1.09 | (0.78-1.52) |
| (95% Confidence Interval) | | | | |
| P value | | | P | = 0.62 |

‡Self-reported History of COPD = “Has a doctor ever told you that you have any of the following conditions . . . ” COPD, Emphysema, Chronic Bronchitis, or Adult Asthma (where prior childhood asthma is excluded).
SNP = Single Nucleotide Polymorphism.
Legend. The clinical risk factors for lung cancer a comparable across the PRS sub-groups with only small differences in smoking intensity, pack year exposure and COPD prevalence related in part to the inclusion of the CHRNA (rs16969968) risk genotype in the 12 SNP panel. The difference in lung cancer mortality is not related to differences in histology, stage or surgery rate across these PRS sub-groups.

### Effect of Combining the Clinical Variables and 12 SNP PRS

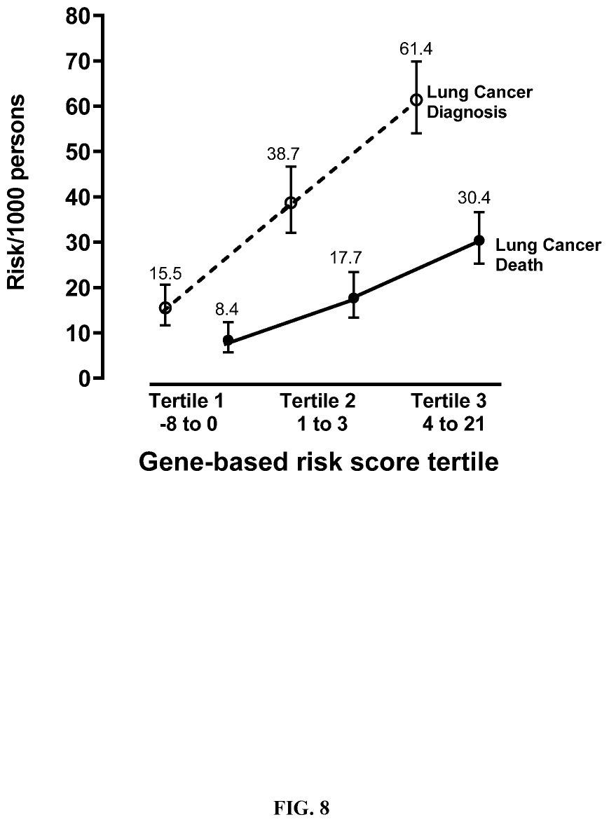

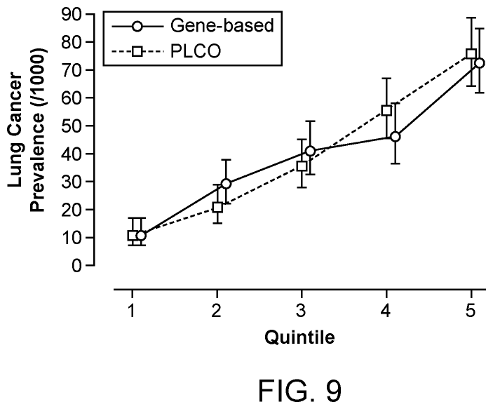

Based on the genotype results from 12 risk genotypes, derived a PRS (range −4 to +6, Table 14) was derived for each participant (N=9,191) and combined it with a clinical score (range −3 to +16) based on 5 clinical variables known to be associated with lung cancer (Table 15). This gene-based composite risk score, derived from previously published studies, was correlated with the 5-year risk of developing lung cancer and dying of lung cancer (FIG. 8 and FIG. 9). Using receiver-operator curve analyses, the area-under-the-curve (AUC) C-statistic was calculated for developing lung cancer or dying of lung cancer (Table 16). When the 12 SNP PRS was added to the clinical score, it was found that the C-statistic for developing lung cancer increased from 0.65 to 0.67 (P<0.0001) and the PRS contribution to the clinical score was significant (P=0.0018). The contribution of adding the clinical score to the PRS was also significant for both developing lung cancer (P<0.0001) and dying of lung cancer (P<0.0001). The gene-based model, which uses only 5 clinical variables, was slightly inferior to the PLCO_{M2012} model (incorporates 11 clinical variables) although confidence intervals were overlapping (FIG. 9). The PLCO_{M2012} was noted to also predict the presence of COPD (Table 16). Using Odds Ratio Estimates to model dying of lung cancer, it was found that each clinical score of 1 added 0.15 increase to the Odds of lung cancer death and each PRS score of 1 added 0.17 increase indicating comparable weighting in the model.

TABLE 15
Algorithm and referencing for the polygenic risk score (PRS)
and clinical score for lung cancer

| | Clinical score | | |
|---|---|---|---|
| | Criteria | Score | |
| Age | ≥65 yo | +4 | |
| Past history of COPD | Yes | +4 | |
| Family history of Lung Cancer | Yes | +3 | |
| Body Mass Index | ≤20 | +4 | |
| | 20-24 | | |
| | ≥25 | −1 | |
| Smoking history | 30-39 pack yrs | −2 | |
| | 40-59 pack yrs | | |
| | ≥60 pack years | +2 | |
| | Polygenic risk score (PRS) | | |
| | Gene (rs #) | Score | |
| 1 | CHRNA (rs16969968) | AA | = +1 |
| 2 | BAT3 (rs1052486) | CC | = +1 |
| 3 | TERT (rs402710) | CC | = +1 |
| 4 | FasL (rs763110) | TT | = −1 |
| 5 | Rev1 (rs3087386) | CC | = −1 |
| 6 | MMP12(rs645419) | GG | = −1 |
| 7 | ITGA11 (rs2306022) | TT/TC | = −1 |
| 8 | IL-6 (rs1800797) | GG | = +1 |
| 9 | IREB (rs2656069) | CC/CT | = −1 |
| 10 | CYP2A6 (rs7937) | TT | = +1 |
| 11 | CRP (rs2808630) | CC | = −1 |
| 12 | AGER (rs2070600) | TT/TC | = +1 |

TABLE 16
Area-under-the-curve analyses and C-statistic for combining
the PRS and clinical scores to derive the composite
gene-based score compared to the PLCOM₂₀₁₂

| | | | | |
|---|---|---|---|---|
| | | | AUC | |
| Risk Model (N = 9191) | Cohort | AUC | (95% CI) | P-value |

1. Gene-based Model

| | | | | |
|---|---|---|---|---|
| Risk of developing lung cancer | | | | |
| 12 SNPs alone (PRS) | All | 0.59 | 0.56-0.62 | <0.0001 |
| add Clinical score | | 0.67 | 0.64-0.70 | <0.0001 |
| Benefit of adding clinical score to | | | | <0.0001 |
| SNP-based PRS score | | | | |
| Clinical score alone | All | 0.65 | 0.62-0.67 | <0.0001 |
| add 12 SNP PRS | | 0.67 | 0.64-0.70 | 0.0001 |
| Benefit of adding SNP-based PRS | | | | P = 0.018 |
| score to clinical score | | | | |
| Risk of dying of lung cancer | | | | |
| 12 SNP PRS alone | All | 0.57 | 0.53-0.61 | 0.0009 |
| add Clinical score | | 0.66 | 0.62-0.70 | <0.0001 |
| Benefit of adding SNP-based PRS | | | | <0.0001 |
| score to clinical score | | | | |

2. Clinical Model Only
PLCO_{M2012} model (N = 9191);
Developing lung cancer-AUC = 0.68 (95% CI = 0.65-0.70), P < 0.0001
Dying of lung cancer-AUC = 0.72 (95% CI = 0.68-0.74), P < 0.0001
Having COPD (GOLD 1-4)-AUC = 0.65 (95% CI = 0.64-0.67),
P < 0.001.
95% CI = 95% Confidence Interval

Comparison with the PLCO_{M2012}—Reclassification According to Gene-Based Risk Model

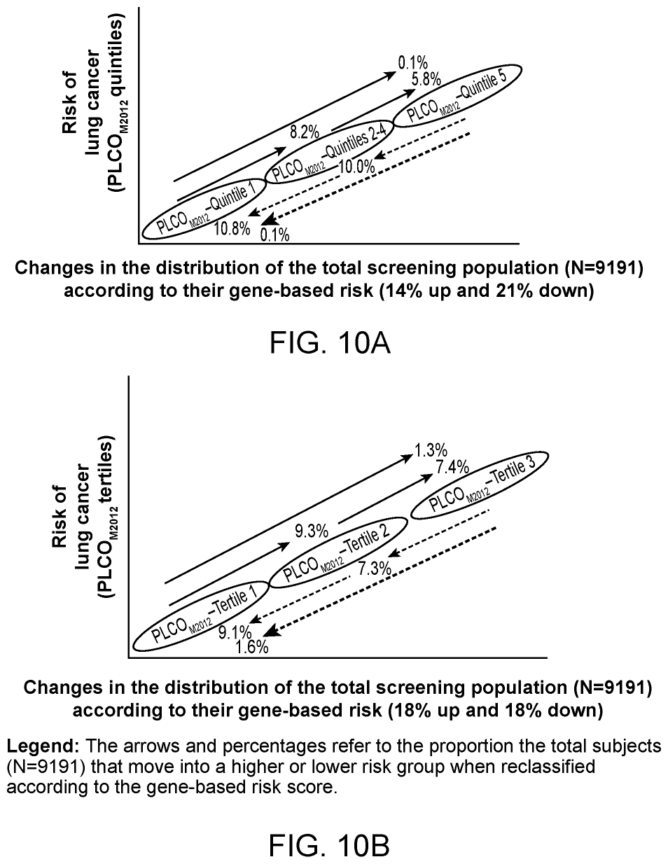

After plotting risk of developing of lung cancer according to the PLCO_{M2012} clinical model according to risk tertiles, each participant's risk was then reclassified by tertile according to the gene-based model. It was found that 1665 NLST participants (18%) moved up a risk tertile and 1647 (18%) moved down a tertile (FIG. 10A). This means that 36% of all screening participants were reclassified into different risk tertiles, when the gene-based risk tool was applied. The effect of adding PRS (SNP data) to the PLCO_{M2012} could not be examined, because the risk models were based on different logistic regression models. These finding show that despite similar predictive utility (overlapping C-statistics, Table 16) according to risk of developing of lung cancer (FIG. 9), the effect of using a different risk algorithm had a considerable effect on the assessment of individualised risk. Using an outcomes-based approach, the proportion of subjects that move between Quintiles 1, Quintiles 2-4 and Quintile 5, after reclassification from PLCO_{M2012} was analysed according to the gene-based model. It was found that 14% moved to a higher group and 21% moved to a lower risk group (FIG. 10B).

Comparison of Screening Outcomes after Screening Randomization

When the outcome of screening before risk stratification was compared with clinical stratification (PLCO_{M2012}) and gene-based stratification (clinical+PRS), it was found that different outcomes according to risk group (Table 17). Compared to the unstratified group (N=9,191) where a 25% reduction in lung cancer mortality was observed in the CT arm (OR=0.75, P=0.067), greater reductions were found in the Quintile 2-4 group according to PLCO_{M2012} (N=5514, 27% reduction, OR=0.73, P=0.15) and gene-based risk testing (N=5663, 43% reduction, OR=0.57, P=0.0044). It was found that while five lung cancer deaths were averted per 1000 screened in the CT arm for the total cohort (N=9191, unstratified) and PLCO_{M2012} Quintiles 2-4 (N=5514), this was 10.3 in Quintile 2-4 for the gene-based models (N=5663, Table 17). This equates to a roughly 2-fold greater efficiency in reducing lung cancer deaths per person screened using the gene-based approach compared to the unstratified and PLCO_{M2012} approaches (Number need to screen to prevent one lung cancer death was 97, 196, and 188 respectively). Using this outcomes-based approach recognises that the greatest efficiency from screening comes from this intermediate risk group, represented by Quintiles 2-4 and also Tertile 2 (Table 14). The relative reduction in lung cancer deaths favouring the CT arm in quintiles 2-4 (intermediate risk group) was 27% for PLCO_{M2012} and 43% for the gene-based approach. This was particularly notable in the gene-based model where in Tertile 2 (62% relative reduction), lung cancer deaths averted per 1000 screened was 15.5 (RR=0.38, P=0.0018) (Table 19). When examined outcomes in Quintiles 1 and 5 from the gene-based models, no benefit (reduction in lung cancer deaths) was found using the latter model (Table 18).

TABLE 17
Screening outcomes following randomization in relative and absolute
terms before and after stratification by PLCO_{M2012} and gene-based
risk approaches for optimized (Quintiles 2-4) screening

| | Screening outcomes favoring the CT arm | | | | | |
|---|---|---|---|---|---|---|
| | Before | | PLCO_{M2012} | | Gene-based | |
| | Stratification | | Stratification | | Stratification | |
| | (Total) | | (Quintiles 2-4) | | (Quintiles 2-4) | |
| | N = 9191 (100%) | | N = 5514 (60%) | | N = 5663 (62%) | |
| Relative reduction in | 25% | | 27% | | 43% | |
| Lung Cancer death (%) | | | | | | |
| Risk ratio (OR) | 0.75 | (0.56, 1.02) | 0.73 | (0.49, 1.10) | 0.57 | (0.38, 0.84) |
| (95% CI) | | | | | | |
| P value | 0.067 | | 0.13 | | 0.0044 | |
| Absolute Lung Cancer deaths | 23 | (−2, 42) | 14 | (−6, 28) | 27 | (11, 41) |
| averted | | | | | | |
| (95% CI) | | | | | | |
| Lung Cancer deaths averted per | −5.1 | (−10.6, 3.9) | −5.3 | (−12.3, 1.6) | −10.3 | (−17.5, −3.2) |

| | | | |
|---|---|---|---|
| 1000 screened | | | |
| (95% CI) | | | |
| NNS to avert one Lung | 196 | 188 | 97^{‡} |
| Cancer death | | | |

95% CI = 95% Confidence Interval. NNS = Number needed to Screen to avert one lung cancer death Legend. The results reported above are derived from data in Table 18 and suggest enrichment for lung cancer amenable to CT-based screening can be achieved in those of intermediate (Quintiles 2-4) risk where using a gene-based approach conferred a two-fold greater efficiency than using PLCOM₂₀₁₂

TABLE 18
Outcomes following randomization for lung cancer (LC) screening stratified according to risk
quintiles for the PLCO_{M2012} and gene-based models

| | PLCO_{M2012} Model | | | | | | | | | |
|---|---|---|---|---|---|---|---|---|---|---|
| | PLCO_{M2012} | | PLCO_{M2012} | | PLCO_{M2012} | | PLCO_{M2012} | | PLCO_{M2012} | |
| | Quintile 1 | | Quintile 2 | | Quintile 3 | | Quintile 4 | | Quintile 5 | |
| | N = 9191 (% total) | | | | | | | | | |
| | N = 1837 | | N = 1838 | | N = 1838 | | N = 1838 | | N = 1840 | |
| | (20.0%) | | (20.0%) | | (20.0%) | | (20.0%) | | (20.0%) | |
| | N = Totals | | | | | | | | | |
| | CT | CXR | CT | CXR | CT | CXR | CT | CXR | CT | CXR |
| | 915 | 922 | 944 | 894 | 903 | 935 | 931 | 907 | 917 | 923 |
| Lung | N = 11 | N = 10 | N = 24 | N = 15 | N = 36 | N = 30 | N = 56 | N = 46 | N = 68 | N = 71 |
| Cancer | (1.2%) | (1.1%) | (2.5%) | (1.7%) | (4.0%) | (3.2%) | (6.0%) | (5.1%) | (7.4%) | (7.6%) |
| Diagnosis | | | | | | | | | | |
| (no PM) | | | | | | | | | | |
| (N = % Grp) | | | | | | | | | | |
| Lung | 9 | 6 | 16 | 3 | 22 | 13 | 28 | 15 | 35 | 33 |
| Cancer | (81.8%) | (60.0%) | (66.7%) | (20.0%) | (61.1%) | (43.3%) | (50.0%) | (32.6%) | (51.5%) | (46.5%) |
| Stage I-II | | | | | | | | | | |
| (N=) | | | | | | | | | | |
| (% LC) | | | | | | | | | | |
| Surgery | 10 | 9 | 17 | 5 | 25 | 15 | 29 | 17 | 35 | 32 |
| (N=) | (91.0%) | (90.0%) | (70.8%) | (33.3%) | (69.4%) | (50.0%) | (51.8%) | (37.0%) | (51.5%) | (45.1%) |
| (% LC) | | | | | | | | | | |
| Lung | 1 | 4 | 8 | 10 | 12 | 14 | 21 | 31 | 30 | 36 |
| Cancer | (9.1%) | (40.0%) | (33.3%) | (66.7%) | (33.3%) | (46.7%) | (37.5%) | (67.4%) | (44.1%) | (50.7%) |
| Death | | | | | | | | | | |
| (N=) | | | | | | | | | | |
| (% LC) | | | | | | | | | | |
| Lung | −3 | | −2 | | −2 | | −10 | | −6 | |
| cancer | | | | | | | | | | |
| deaths | | | | | | | | | | |
| averted | | | | | | | | | | |
| favoring | | | | | | | | | | |
| the CT | | | | | | | | | | |
| arm | | | | | | | | | | |
| Lung | −3 | | −14 | | | | | | −2 | |
| cancer | | | | | | | | | | |
| deaths | | | | | | | | | | |
| averted | | | | | | | | | | |
| favoring | | | | | | | | | | |
| the CT | | | | | | | | | | |
| arm Q2- | | | | | | | | | | |
| Q4 | | | | | | | | | | |
| Relative | | | 25.5% reduction with CT in Quintiles 2-4 | | | | | | | |
| reduction | | | (24.7% reduction over Quintiles 1-5) | | | | | | | |
| in lung | | | | | | | | | | |
| cancer | | | | | | | | | | |
| mortality | | | | | | | | | | |
| | Gene-based Model | | | | | | | | | |
| | Gene-based | | Gene-based | | Gene-based | | Gene-based | | Gene-based | |
| | Quintile 1 | | Quintile 2 | | Quintile 3 | | Quintile 4 | | Quintile 5 | |
| | N = 9191 (% total) | | | | | | | | | |
| | N = 2076 | | N = 1832 | | N = 1767 | | N = 2064 | | N = 1452 | |
| | (22.6%) | | (19.9%) | | (19.2%) | | (22.5%) | | (15.8%) | |
| | N = Totals | | | | | | | | | |
| | CT | CXR | CT | CXR | CT | CXR | CT | CXR | CT | CXR |
| | 997 | 1079 | 937 | 895 | 897 | 870 | 1057 | 1007 | 722 | 730 |
| Lung | 15 | 9 | 26 | 28 | 35 | 38 | 56 | 42 | 63 | 55 |
| Cancer | (1.5%) | (0.83%) | (2.8%) | (3.1%) | (3.9%) | (4.4%) | (5.3%) | (4.2%) | (8.7%) | (7.5%) |
| Diagnosis | | | | | | | | | | |
| (no PM) | | | | | | | | | | |
| (N = % Grp) | | | | | | | | | | |
| Lung | 8 | 3 | 15 | 9 | 23 | 17 | 30 | 11 | 34 | 30 |
| Cancer | (53.3%) | (33.3%) | (57.7%) | (32.1%) | (65.7%) | (44.7%) | (53.6%) | (26.2%) | (53.9%) | (54.5%) |
| Stage I-II | | | | | | | | | | |
| (N=) | | | | | | | | | | |
| (% LC) | | | | | | | | | | |
| Surgery | 8 | 5 | 16 | 12 | 24 | 16 | 35 | 14 | 33 | 31 |
| (N % LC) | (53.3%) | (55.6%) | (61.5%) | (42.9%) | (68.6%) | (42.1%) | (62.5%) | (33.3%) | (52.4%) | (56.4%) |
| Lung | 7 | 3 | 8 | 20 | 11 | 21 | 20 | 25 | 26 | 26 |
| Cancer | (46.7%) | (33.3%) | (30.8%) | (71.4%) | (31.4%) | (55.3%) | (35.7%) | (59.5%) | (41.3%) | (47.3%) |
| Death | | | | | | | | | | |
| (N=) | | | | | | | | | | |
| (% LC) | | | | | | | | | | |
| Lung | +4 | | −12 | | −10 | | −5 | | 0 | |
| cancer | | | | | | | | | | |
| deaths | | | | | | | | | | |
| averted | | | | | | | | | | |
| favoring | | | | | | | | | | |
| the CT | | | | | | | | | | |
| arm | | | | | | | | | | |
| Lung | +4 | | −27 | | | | | | 0 | |
| cancer | | | | | | | | | | |
| deaths | | | | | | | | | | |
| averted | | | | | | | | | | |
| favoring | | | | | | | | | | |
| the CT | | | | | | | | | | |
| arm in | | | | | | | | | | |
| Q2-Q4 | | | | | | | | | | |
| Relative | | | 41% reduction with CT in Quintiles 2-4 | | | | | | | |
| reduction | | | (24.1% reduction over Quintiles 1-5) | | | | | | | |
| in lung | | | | | | | | | | |
| cancer | | | | | | | | | | |
| mortality | | | | | | | | | | |

Legend.
The gene-based model achieves greater reduction in lung cancer mortality from randomization to the CT arm in the intermediate risk (Quintiles 2-4) subgrouping suggesting enrichment for lung cancer most amenable to lung cancer survival after CT-based screening. This enrichment in the Quintiles 2-4 favoring CT for the gene-based model appears to be related to a greater proportion of stage 1-2 disease, greater surgical rates for lung cancer and greater lung cancer deaths averted relative to the CXR arm.

TABLE 19
Outcomes following randomization for lung cancer (LC) screening stratified according
to risk tertiles for the PLCOM2012 and gene-based models

| | | | | Screening outcome favoring CT | | | |
|---|---|---|---|---|---|---|---|
| | | | | | Risk ratio for | | |
| N = 9191. | | | | Absolute LC | LC death^{†} | LC deaths averted^{‡}/ | P |
| Risk models | Tertile | CT | CXR | deaths averted^{‡} | (95% CI) | 1000 screened | value^{†} |
| PLCO_{M2012} | | | | | | | |
| Model | | | | | | | |
| Total Subjects | 1 | 1533 | 1530 | −5 | 0.58 | −3.3 | 0.26 |
| Total LC | | 28 | 22 | | (0.23, 1.48) | (−8.8, 2.3) | |
| diagnosed | | 7 | 12 | | | | |
| LC deaths | | | | | | | |
| Total Subjects | 2 | 1541 | 1523 | −6 | 0.75 | −4.1 | 0.35 |
| Total LC | | 59 | 49 | | (0.42, 1.36) | (−12.5, 4.3) | |
| diagnosed | | 19 | 25 | | | | |
| LC deaths | | | | | | | |
| Total Subjects | 3 | 1536 | 1528 | −5 | 0.91 | −3.5 | 0.62 |
| Total LC | | 108 | 101 | | (0.64, 1.30) | (−17.0, 10.1) | |
| diagnosed | | 56 | 61 | | | | |
| LC deaths | | | | | | | |
| Gene-based | | | | | | | |
| model | | | | | | | |
| Total Subjects | 1 | 1446 | 1518 | +2 | 1.24 | 1.7 | 0.60 |
| Total LC | | 25 | 21 | | (0.55, 2.76) | (−4.7, 8.2) | |
| diagnosed | | 13 | 11 | | | | |
| LC deaths | | | | | | | |
| Total Subjects | 2 | 1385 | 1326 | −20 | 0.38 | −15.5 | 0.0018 |
| Total LC | | 51 | 54 | | (0.20, 0.71) | (−25.3, −5.7) | |
| diagnosed | | 13 | 33 | | | | |
| LC deaths | | | | | | | |
| Total Subjects | 3 | 1779 | 1737 | −5 | 0.88 | −3.5 | 0.53 |
| Total LC | | 119 | 97 | | (0.59, 1.31) | (−14.3, 7.3) | |
| diagnosed | | 46 | 51 | | | | |
| LC deaths | | | | | | | |

^{†}Lung cancers (LC) diagnosed at post-mortem were excluded from the screening outcomes analysis.
95% CI = 95% Confidence Interval.
^{‡}LC deaths averted favoring the CT arm over the CXR arm.
Legend.
The gene-based model achieves greater reduction in lung cancer mortality from randomization to the CT arm in the intermediate risk (Tertile 2) subgroup suggesting enrichment for lung cancer most amenable to lung cancer survival after CT-based screening.

### Discussion

In this clinical validity study from a subgroup of the NLST, it was reported that while adding genetic data from a 12 SNP PRS improved the risk assessment for developing lung cancer, this composite gene-based approach to screening selection was nearly 2-fold more efficient than an existing clinical model in identifying those most likely to benefit from lung cancer screening. It was also found that this gene-based approach was associated with considerable re-classification of risk not reflected in the marginal improvement in the area-under-the-curve analysis. This approach might result from including the 12 SNP PRS previously linked to greater lung cancer mortality and the recognition that genetic susceptibility might have a role to play in the outcomes from screening. Lastly the gene-based approach to assessing risk of lung cancer also provides an assessment of the likely benefit of screening. Consistent with recent analyses of screening cohorts that have considered the effects of comorbid disease, reduced life expectancy and personal preference on outcomes of screening, it was found that those at intermediate risk gain the most from screening. This means that screening participants, or their referring physicians, can assess the likely outcomes from screening at a personalized level based in part on the individual's genetic risk. It was suggested that this outcomes-based approach might outperform that based on risk alone, where risk and benefit are not linearly related at the upper end of the risk spectrum.

In the recent era of molecular genetics, it was shown that the genetic susceptibility to lung cancer overlaps with that of COPD through shared SNP-based associations. The 12 SNP lung cancer panel has been validated in a previous study where the association with lung cancer and/or COPD has been able to be distinguished. Although 3 of the risk genotypes in this 12 SNP panel overlap with COPD risk, the composite genetic score PRS did not itself correlate with any known clinical risk variables with lung cancer although a weak signal for smoking intensity (one cigarette/day) and COPD prevalence (3% difference) was observed. The 12 SNP PRS has been associated with lung cancer mortality relative to total mortality and may reflect lung cancer lethality through some as yet unknown pathogenic processes. This was shown in the greater improvement in the AUC statistic relative to that derived for developing lung cancer. Given the outcome of lung cancer screening results from a complex interplay between the individual's health and the biology of their lung cancer, it was expected that biomarkers reflecting the latter might combine with those reflecting the former to enhance the ability to predict outcomes of screening. While it is possible that the genetic risk, that includes COPD-related risk genotypes, might be reflecting a greater propensity to COPD conferring worse outcomes (more aggressive lung cancer), this 12 SNP PRS did not correlate with any risk variables including lung function. It is notable that for the majority of the risk genotypes conferring reduced or enhanced risk of lung cancer (10/12), the effect was strongest for lung cancer in the absence of COPD. It was shown that this group has a nearly two-fold greater reduction in lung cancer deaths with screening and might relate in part to underlying lung cancer biology. This suggests that genetic risk for developing lung cancer in screening participants without pre-existing COPD might play an important role in screening outcomes. Further lung cancer studies incorporating the widespread use of spirometry will be needed to confirm this finding.

There is a growing realization that screening for lung cancer in older heavier smokers involves more complexity than existing screening approaches for breast and colon cancer. First, this target screening population has considerably more underlying comorbid disease at baseline and in particular a high prevalence of COPD. Post-hoc analyses of data from the NLST shows that existing risk models for lung cancer such as the PLCO_{M2012}, based on well validated risk variables, also predict a greater tendency to COPD. This is important as COPD goes largely undiagnosed, particularly in those with mild-to-moderate disease. Those with more advanced forms of COPD (severe-to-very severe), while at greatest risk for lung cancer, gain little from screening due to several competing factors including lower surgical rates, more non-lung cancer deaths and reduced life expectancy (greater biological age). This means the risk to benefit relationship in screening for lung cancer is attenuated for those at greatest risk and that selection for lung cancer screening should not be based or optimized on risk alone. There is a growing interest in optimizing screening for lung cancer based on subgroups who achieve the best outcome from screening, and this appears to be those at greater risk but reduced comorbid disease. This outcomes based approach has been shown to be more cost-effective and better suit participant preference for screening. It might also form an important part of the decision making around initiating or continuing lung cancer screening in those at the extreme of the risk spectrum.

An approach to this study has been the identification and inclusion of genetic factors underlying both lung cancer and COPD. Despite this, the composite PRS did not correlate with COPD, severity of airflow limitation or indeed smoking exposure (Table 16). This suggests that the addition of the 12 SNP PRS helped to meaningfully reclassify risk by including genetic susceptibility to the overall risk assessment independent of existing clinical variables. The effect of adding the PRS was assessed using traditional AUC and NRI approaches although many advocate a more outcomes-based approach to assessing the clinical utility of this gene-based risk assessment. In this approach, it is the improvement in clinical usefulness that determines whether this gene-based approach to screening has clinical utility not just improvements in prediction accuracy (AUC). In this regard, it was found that this gene-based test achieved a two-fold greater reduction in lung cancer deaths than one of the best validated clinical models. While the cost-effectiveness of these two approaches is currently being investigated, because genetic testing is now so cheap, greater cost-effectiveness is very likely.

The modest number of lung cancers reflects the challenge of prospective studies of lung cancer and further studies are planned. The full NLST enrolled over 53.000 high risk smokers and after a mean of 6.4 years of follow up compared results for about 2.000 lung cancer cases across the two screening arms. The 20% reduction in lung cancer deaths came down to a difference of just 76 less deaths in those randomised to the CT arm. Strengths of this study include: confirming clinical utility in a highly representative screening population, that genotyping was done blind to screening outcomes and targeted well validated SNPs, and that the algorithm combining risk genotypes made no assumptions about effect size or gene-gene interaction. Furthermore, the risk genotypes were pre-specified and captured both protective effects (reduced risk) and susceptibility effects (increased risk), including SNP variants relevant in COPD. Another improvement is that algorithmic approach combining personalised genetic data deriving the PRS was also pre-specified. Lastly, the PRS was able to be correlated with who died of their lung cancer in the context of screening rather than just who developed lung cancer in the setting of a case-control study. A novel approach to this study has been the identification and inclusion of genetic factors underlying both lung cancer and COPD. Despite this, the composite PRS did not strongly correlate with COPD, severity of airflow limitation or indeed smoking exposure (Table 15). This suggests that the addition of the 12 SNP PRS helped to meaningfully reclassify risk by including genetic susceptibility to the overall clinical risk assessment independent of existing clinical variables. The effect of adding the PRS was assessed using traditional AUC analyses although many advocates a more outcomes-based approach to assessing the clinical utility of the gene-based risk assessment tool. In this regard, it is the improvement in clinical usefulness that determines whether this gene-based approach to screening has clinical utility not just improvements in prediction accuracy. In this regard, the gene-based test achieved a two-fold greater reduction in lung cancer deaths and 2-fold greater efficiency than one of the best validated clinical models (PLCO_{M2012}). While the cost-effectiveness of the gene-based approach is currently being investigated, because genetic testing is now so cheap, greater cost-effectiveness is very likely.

In summary, in the current study, a composite gene-based risk score was compiled for developing of lung cancer, that reclassifies risk for over 40% of screening subjects based on clinical variables alone, and better predicts outcomes of screening. Such as approach incorporates a simple one-off and inexpensive test that may help to predict whether a screening participants is likely to benefit from screening. Such a tool may be of interest to an “at risk smoker” otherwise eligible for funded screening or considering the merits of unfunded screening. This tool may also be of interest to a medical team helping to advise on the benefits of screening in the shared decision-making stage. The gene-based approach to selecting screening participants may have cost-effective benefits by reducing the number needed to screen (NNS) to prevent one lung cancer. This outcome-based approach deviates from existing approaches which recommends targeting those at greatest risk (risk-based selection) for lung cancer screening. It was suggested that an outcomes-based approach to lung cancer screening may be of greater utility.

### Example 5. Polygenic Risk Score Identifies More Lethal Lung Cancers in the National Lung Screening Trial: A Prospective Clinical Validity Study

Introduction: Epidemiological studies suggest lung cancer results from the combined effects of smoking and genetic susceptibility. The clinical utility of polygenic risk scores (PRS), derived from combining the results from multiple germline genetic variants, have been explored in breast and prostate cancer.

Epidemiological studies have identified a number of clinical risk variables underlying lung cancer susceptibility and these have been combined in models such as the PLCO_{M2012} to derive a composite risk for future lung cancer. These risk models have been validated in independent cohorts to a variable degree and performance characteristics. Single nucleotide polymorphisms (SNPs) may be associated with lung cancer. The presence of chronic obstructive pulmonary disease (COPD) may be relevant to susceptibility to lung cancer independent of age and smoking exposure.

This was a post-hoc analysis of 9,191 Non-Hispanic white subjects from the National Lung Screening Trial (NLST), a sub-study of high risk smokers randomised to annual compute tomography (CT) or chest x-ray (CXR) and followed for an average of 6.4 years. This study examined the relationship between a composite polygenic risk score (PRS) calculated from 12 validated risk genotypes and outcomes during this study. The baseline characteristics and screening outcomes were compared according to low (N=2813, 31%), intermediate/neutral (N=2504, 27%) or high (N=3874, 42%), PRS scores.

In this prospective clinical validity study, it was found that PRS correlated with lung cancer incidence (P<0.0001) and mortality (P=0.004). In an adjusted multivariable logistic regression analysis, PRS was independently associated with lung cancer death (P=0.0027). Screening participants with intermediate and high PRS had higher lung cancer mortality, relative to those with a low PRS score (Rate ratios=1.73 (95% CI 1.14-2.64, P=0.010) and 1.89 (95% CI 1.28-2.78, P=0.009) respectively. This was despite comparable baseline demographics and comparable lung cancer characteristics. Following screening, the odds ratio of lung cancer lethality in the CXR arm relative to CT was 1.57 (95% CI 0.61-4.04, P=0.35) for a low score and 2.32 (95% CI 1.15-3.70, P=0.0005) in the combined neutral/high score group. Adding 12 SNP PRS to a clinical risk score improved the area-under-the-curve (0.65 to 0.67, P=0.018).

In this biomarker-based cohort study, an elevated PRS was associated with both greater lung cancer lethality and mortality, and may represent a novel biomarker of outcome following screening.

Key Objective: The clinical utility of a polygenic risk score (PRS) in cancer screening is of growing interest but to date has not been examined in the context of lung cancer. While outcomes from screening result from a complex relationship involving both patient and lung cancer.

It was found that lung cancer PRS helps predict the development of lung cancer and who may die from their lung cancer (clinical validity). The increased risk of dying of lung cancer conferred by an elevated PRS was found to be independent of the patient's clinical risk variables (smoking, age and comorbidity), lung cancer characteristics (histology, stage and surgery), screening arm and competing causes of death.

Through candidate gene and genome wide association studies (GWAS), several well replicated associations between SNP variants and lung cancer have been reported. More recently, there has been a growing interest in combining germline genetic data to derive personalised composite risk scores (termed polygenic risk score or PRS). These risk scores are generally based on simple algorithms combining allele or genotype counts according to genotyping results and have been reported to correlate with breast and prostate cancer outcomes.

This current study examines the utility of a validated 12 SNP panel in the context of a lung cancer screening trial, to determine its effect on lung cancer lethality and mortality.

### Methods

### Subjects

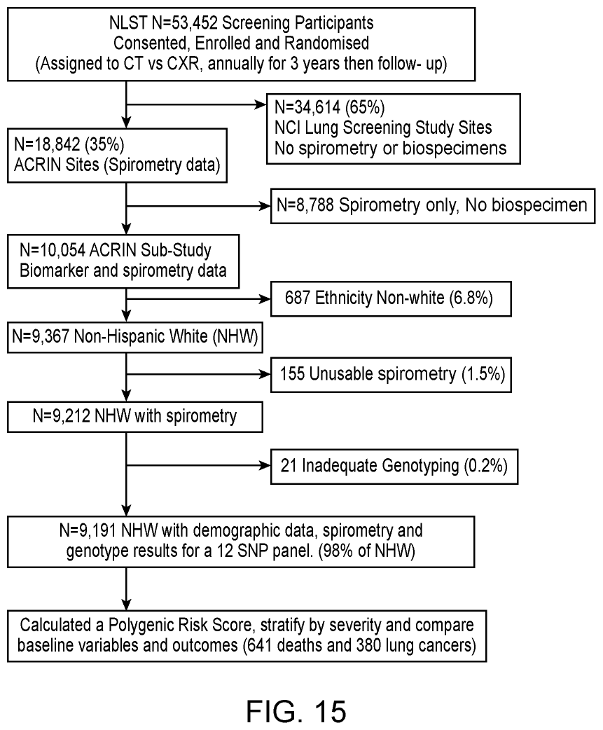

This is a secondary data analysis of the National Lung Screening Trial (NLST). The recruitment and study design of this trial, involving 53,452 screening participants yielding 2058 histology confirmed lung cancers, has been described elsewhere. In the American College of Radiology Imaging Network (ACRIN) sub-cohort of the NLST, participants from 23 US-based centres agreed to undergo baseline pre-bronchodilator spirometry and, for a subgroup (FIG. 1), blood sampling for biomarker analysis (N=10,054). Demographic data, including history of pre-morbid disease, were collected through an extensive questionnaire. From the total group (all ethnicities), genomic data for Non-Hispanic whites was found, comprising 9,367 high risk smokers from which 380 lung cancers were diagnosed during the study follow up (FIG. 15).

### Pulmonary Function Testing

In the NLST-ACRIN cohort, pre-bronchodilator spirometry was measured at baseline screening (TO) in the majority of participants according to a previously published protocol. The spirometry was measured by trained staff using a Spiropro spirometer (eResearchTechnology, GmbH, Germany). The severity of airflow limitation was defined according to the Global Initiative on Chronic Obstructive Lung Disease (GOLD) criteria grades 1-4 (www.GOLD.org accessed Mar. 2, 2021).

### Lung Cancer Outcomes

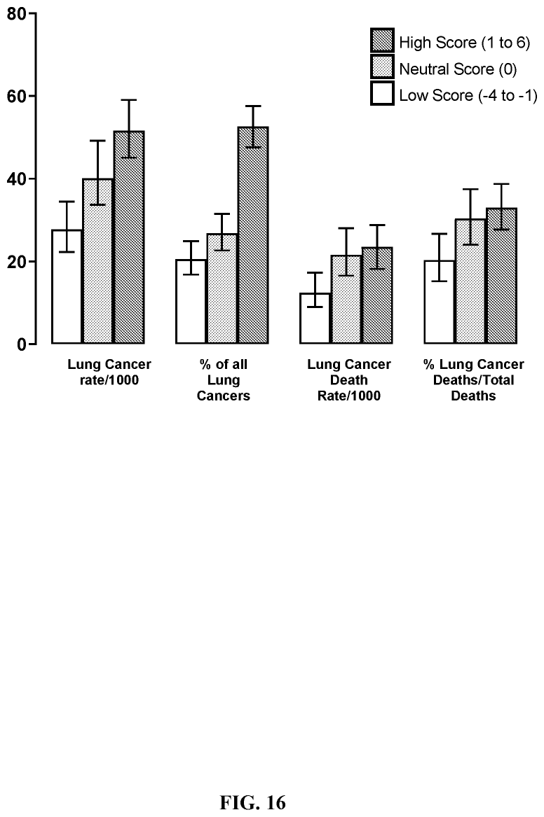

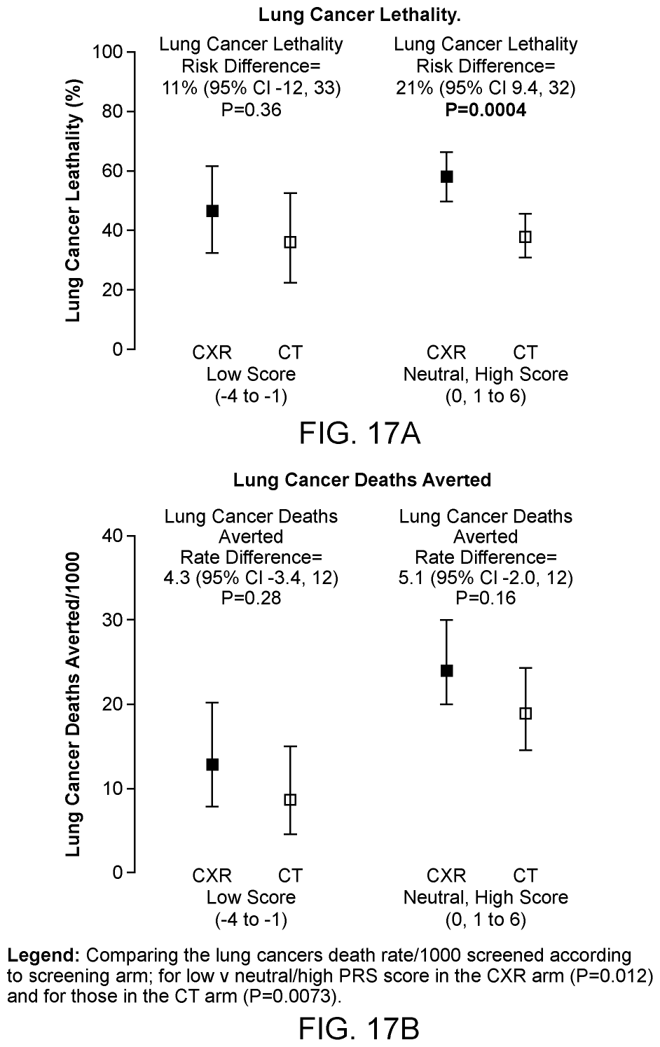

Lung cancer cases included all those diagnosed during the trial (N=380), whether screen or non-screen detected (interval), or prevalent (diagnosed at TO or during the first year) or incident lung cancers (diagnosed during subsequent years T1 to T6) or at post-mortem. All lung cancer cases were confirmed on histological sampling according to accepted international classification criteria. Lung function results and mortality outcomes were available for all 380 lung cancer cases (100% of total). Cause of death was ascertained through review of clinical records and death certification. The relationship between lung cancer PRS and lung cancer mortality was examined as the percentage of lung cancer deaths/all lung cancers in the group (lung cancer “lethality”), percentage of all lung cancers deaths/all deaths, lung cancer deaths per 1000 screened and risk/rate ratios, both before and after screening randomisation (Table 21, FIGS. 16-17, FIG. 17).

### Genotyping and Algorithms

Genomic DNA was extracted from buffy coat samples using standard salt-based methods and purified genomic DNA was aliquoted (10 ng·μL⁻¹ concentration) into 384-well plates. DNA concentration and purity were determined by using Nanodrop spectrophotometry. Genotyping entailed using the Sequenom™ system (Sequenom™ Autoflex Mass Spectrometer and Samsung 24 pin nanodispenser) by Agena (Agena BioScience, San Diego, CA, USA) multiplexed into 2 assays (Agena MassARRAY Assay Design 3.0). The Sequenom™ sequences were designed in house by Agena with amplification and separation methods (iPLEX™, www.sequenom.com) as previously described. Based on prior studies in the literature, the risk genotype/s were pre-specified and assigned as susceptible (Odds >1.0) or protective (Odds <1.0). A call rate for each SNP of >98% was achieved. Replication using this method was undertaken in 2,000 random samples and achieved 99% accuracy for genotyping across the 12 SNPs. Using a pre-specified algorithm, the sum of 6 susceptible genotypes (+1 for each) was added to the 6 protective genotypes (−1 for each) deriving the PRS (range −4 to +6).

### Statistical Analysis

Differences between groups for continuous variables were sought using analysis of variance. Significant differences were further explored as appropriate using Tukey's post hoc method. Differences between groups for categorical variables were sought using chi-squared test (or exact methods where appropriate). Multivariable logistic regression (PRS as a continuous variable) and General linear modelling (binary distribution, logit link function) was used to estimate the odds of lung cancer death, with and without adjustment for clinical risk variables (PLCO_{M2012} model). Mid-P Exact risk and rate differences were used to assess the magnitude and direction of the association (www.openpepi.com accessed 25/03/2021). Statistical significance was defined as a two tailed P<0.05 for comparisons between PRS groups. No adjustment for multiplicity was performed. All planned comparisons are presented in the tables of this paper. All analyses were performed using SAS (V 9.4, SAS Institute Inc, Cary NC) or STATA statistical software.

### Results

Based on the genotype results from 12 independent risk genotypes, a PRS was calculated for each participant (N=9191) and sub-grouped according to low (scores −4 to −1, predominantly protective genotypes), intermediate (neutral score of zero, equal protective and susceptible genotypes) and high (scores 1 to 6, predominantly susceptible genotypes) that encompassed 31% (n=2813), 27% (N=2504) and 42% of subjects (N=3874), respectively (crude tertiles). The demographic data for these subgroups are summarised in Table 20. The groups were not significantly different for most of the baseline demographic variables associated with risk of lung cancer specifically, age, sex, smoking status, years smoked, years quit, family history of lung cancer, self-reported history of COPD, BMI, education and pre-existing comorbidities. Increasing PRS was associated with slightly greater cigarettes per day and pack years but the differences across these risk groups were small (1 cigarette/day and 1 pack year). Despite comparable lung function, higher PRS was associated with slightly greater spirometry-defined COPD (GOLD 1-4) although again the differences were very small (33% vs 36%, P=0.02) (Table 20).

TABLE 20
Lung cancer risk factors and demographic variables according to a
12 SNP polygenic risk score (PRS) severity.

| | Polygenic Risk Score Groups (absolute score range) | | | | | | |
|---|---|---|---|---|---|---|---|
| | Low Score | | Neutral Score | | High Score | | |
| | (−4 to −1) | | 0 | | (1 to 6) | | P value |
| N = 9,191 (% total) NHW | 2813 | (30.6%) | 2504 | (27.2%) | 3874 | (42.1%) | |
| Demographics | | | | | | | |
| Age-mean (SD) | 61.7 | (5.2) | 61.9 | (5.1) | 61.8 | (5.1) | 0.40 |
| Sex-% Male | 1608 | (57.2%) | 1428 | (57.0%) | 2190 | (56.5%) | 0.86 |
| Smoking Status-Current Smoker | 1374 | (48.8%) | 1161 | (46.4%) | 1882 | (48.6%) | 0.14 |
| Pack Years-mean (SD) | 55.2 | (22.7) | 55.7 | (23.2) | 57.1 | (23.7) | 0.0021 |
| Cigarettes/day-mean (SD) | 27.7 | (10.8) | 28.0 | (10.9) | 28.7 | (11.3) | 0.0002 |
| Years Quit mean (SD) | 3.6 | (5.0) | 3.8 | (5.1) | 3.8 | (5.1) | 0.40 |
| Smoking duration years mean (SD) | 40.3 | (7.1) | 40.4 | (7.5) | 40.3 | (7.6) | 0.93 |
| Family history of Lung Cancer-yes | 671 | (23.9%) | 607 | (24.2%) | 912 | (23.5%) | 0.81 |
| (%) | | | | | | | |
| Self-reported Hx of COPD (%)‡ | 561 | (19.9%) | 508 | (20.3%) | 819 | (21.1%) | 0.46 |
| BMI mean (SD) | 27.8 | (5.0) | 27.9 | (5.2) | 27.9 | (5.1) | 0.87 |
| Education Level-N (%) | | | | | | | |
| High School or less | 809 | (28.8%) | 677 | (27.0%) | 1138 | (29.4%) | 0.05 |
| Post High School/Some College- | 995 | (35.4%) | 912 | (36.4%) | 1359 | (35.1%) | |
| College graduate | 508 | (18.1%) | 403 | (16.1%) | 661 | (17.1%) | |
| Postgraduate/Professional | 423 | (15.0%) | 453 | (18.1%) | 617 | (15.9%) | |
| Other/unknown | 78 | (2.8%) | 59 | (2.4%) | 99 | (2.6%) | |
| Lung Function | | | | | | | |
| FEV₁/FVC-mean (SD) | 71.4 | (10.7) | 70.93 | (10.9) | 70.8 | (10.6) | 0.057 |
| FEV₁% predicted-mean (SD) | 81.9 | (19.4) | 81.4 | (20.5) | 80.9 | (20.2) | 0.15 |
| Total COPD GOLD 1-4 | 925 | (32.9%) | 895 | (35.7%) | 1394 | (36.0%) | 0.02 |
| Pre morbid conditions (self-report) | | | | | | | |
| COPD | 182 | (6.5%) | 195 | (7.8%) | 285 | (7.4%) | 0.16 |
| Chronic Bronchitis | 327 | (11.6%) | 254 | (10.1%) | 442 | (11.5%) | 0.18 |
| Emphysema | 232 | (8.2%) | 237 | (9.5%) | 378 | (9.8%) | 0.10 |
| Asthma-adult | 181 | (6.4%) | 171 | (6.8%) | 272 | (7.0%) | 0.60 |
| Pneumonia | 772 | (27.4%) | 709 | (28.3%) | 1099 | (28.4%) | 0.67 |
| Heart Disease | 392 | (13.9%) | 312 | (12.5%) | 521 | (13.4%) | 0.28 |
| Hypertension | 1002 | (35.6%) | 890 | (35.5%) | 1374 | (35.5%) | 0.99 |
| Stroke | 91 | (3.2%) | 66 | (2.6%) | 109 | (2.8%) | 0.40 |
| Diabetes | 243 | (8.6%) | 251 | (10.0%) | 342 | (8.8%) | 0.16 |
| Any cancer History | 121 | (4.3%) | 97 | (3.9%) | 163 | (4.2%) | 0.71 |

‡Self-reported History of COPD = “Has a doctor ever told you that you have any of the following conditions . . . ” COPD, Emphysema, Chronic Bronchitis, or Adult Asthma (where prior childhood asthma is excluded). COPD = Chronic Obstructive Pulmonary Disease.

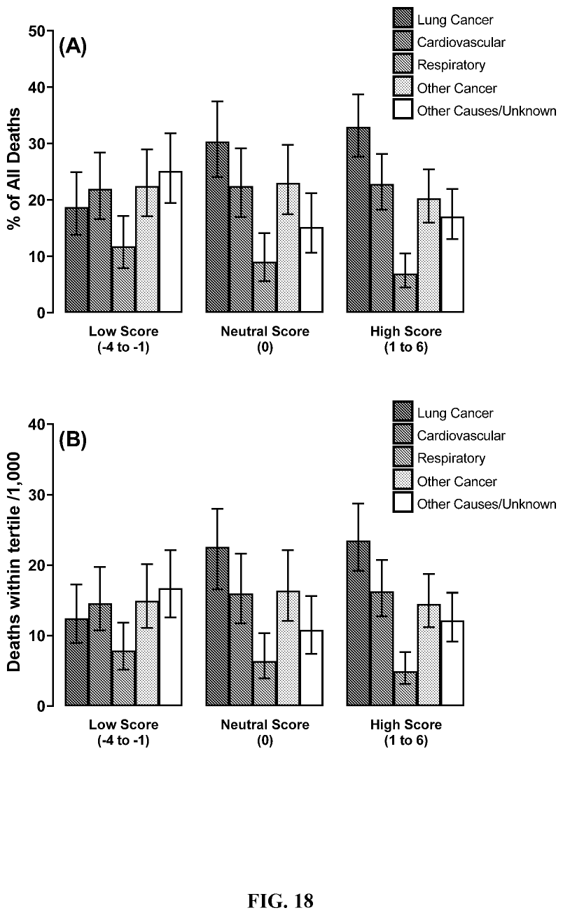

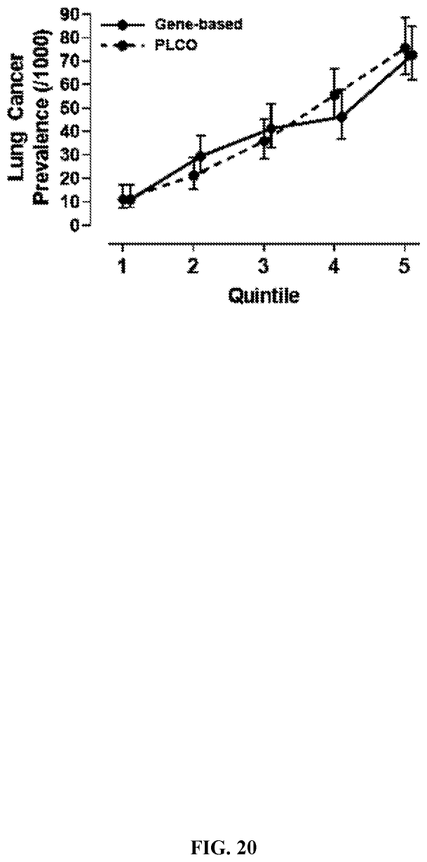

Increasing PRS was associated with a linear increase in lung cancer incidence, absolute lung cancer deaths and lung cancer deaths as a proportion of all deaths (Table 21 and FIG. 15). Notably, subjects in the intermediate (Neutral) and high risk PRS sub-groups had similar lung cancer mortality (Mortality rate ratios, Table 21), which were 1.7 and 1.9 fold greater respectively (P=0.01 and P=0.0009) than those in the low PRS group (Table 21, FIG. 15). These findings were evident despite very similar lung function, histology, stage, detection method, surgical rates and diagnostic screening interval (Table 22). When cause-specific deaths were compared according to increasing PRS, only lung cancer mortality demonstrated a positive relationship (Table 21, FIG. 17). After considerable adjustment for various clinical variables, it was found that the lung cancer mortality remained significantly greater in the high versus low PRS groups (FIG. 18) and that PRS, as continuous variable, remained an independent predictor of lung cancer death in the multivariable model (Table 23). After randomisation, screening participants in the combined neutral/high PRS group randomised to CXR had the greatest lung cancer lethality (21% higher than CT, P=0.0004, Table 21, FIG. 16(a)). This was in conjunction with comparable surgical rates favouring those randomised to CT screening over CXR (15%, P=0.21 and 14%, P=0.019 for low versus neutral/high groups respectively) and comparable lung cancer characteristics (Table 22). The risk difference for lung cancer deaths averted after randomisation was significantly higher in the neutral/high PRS (versus low PRS) across screening arm (FIG. 16(b)), but not significantly different between CT and CXR across PRS subgroups (21% vs 34%) (Table 21). FIG. 20 depicts combining a gene-based risk algorithm with the PRS to derive a composite individualised gene-based risk score for developing lung cancer (Table 24). The predefined scoring algorithm for the clinical variables are: age ≥65 years old (+4), history of COPD (+4), Family history (1st degree) of lung cancer (+3), abnormal BMI (BMI ≤20=+4 and BMI ≥25=−1), smoking pack years (pack years ≤39=−2 and pack years ≥60=+2).

TABLE 21
Overall mortality and reduction in lung cancer deaths after screening according to polygenic risk
score (PRS) groups (Low, Neutral or High).

| | Polygenic Risk Score Groups (absolute score range) | | | | | |
|---|---|---|---|---|---|---|
| | Low score | | Neutral Score | | High score | |
| | (−4 to −1) | | 0 | | (1 to 6) | |
| N = 9,191 NHW (% of total cohort) | 2813 | (30.6%) | 2504 | (27.2%) | 3874 | (42.1%) |
| Lung Cancer | | | | | | |
| Total lung cancer^{‡} (% total, N = 380) | 78 | (20.5%) | 102 | (26.8%) | 200 | (52.6%) |
| Incidence/per 1000 ^{Φ}(95% CI) | 27.7 | (22.3, 34.5) | 40.7 | (33.7, 49.2) | 51.6 | (45.1, 59.1) |
| Overall Mortality (prevalence) | | | | | | |
| Total Deaths (%, per 100) | 187 | (6.6%) | 178 | (7.1%) | 276 | (7.1%) |
| % and 95% CI all deaths (N = 641) | 29.2%, | (25.8, 32.9) | 27.8% | (24.4, 31.4) | 43.1% | (39.3, 46.9) |
| Lung Cancer (LC) Deaths (%, per 100) | 35/187 | (18.7%) | 54/178 | (30.3%) | 91/276 | (33.0%) |
| % all deaths (N = 641) | 5.5% | (4.0, 7.5) | 8.4% | (6.5, 10.8) | 14.2% | (11.7, 17.1) |
| % all deaths by tertile | 1.2% | (0.90, 1.73) | 2.2% | (1.7, 2.8) | 2.3% | (1.9, 2.9) |
| Cardiovascular (CVD) Deaths | 41 | (21.9%) | 40 | (22.5%) | 63 | (22.8%) |
| % all deaths (N = 641) | 6.4% | (4.8, 8.6) | 6.2% | (0.2, 1.6) | 9.8% | (7.8, 12.4) |
| % all deaths by tertile | 1.5% | (1.1, 2.0) | 1.6% | (1.2, 2.2) | 1.6% | (1.3, 2.1) |
| Respiratory Deaths | 22 | (11.8%) | 16 | (9.0%) | 19 | (6.9%) |
| % all deaths (N = 641) | 3.4% | (2.3, 5.1) | 2.5% | (1.5, 4.0) | 3.0% | (1.9, 4.6) |
| % all deaths by tertile | 0.78% | (0.52, 1.18) | 0.64% | (0.39, 1.04) | 0.49% | (0.31, 0.76) |
| Other Cancer Deaths | 42 | (22.5%) | 41 | (23.0%) | 56 | (20.3%) |
| % all deaths (N = 641) | 6.6% | (4.9, 8.7) | 6.4% | (4.8, 8.6) | 8.7% | (6.8, 11.2) |
| % all deaths by tertile | 1.5% | (1.1, 2.0) | 1.6% | (1.2, 2.2) | 1.4% | (1.1, 1.9) |
| Other Deaths or COD unknown | 47 | (25.1%) | 27 | (15.2%) | 47 | (17.0%) |
| % all deaths (N = 641) | 7.3% | (5.6, 9.6) | 4.2% | (2.9, 6.1) | 7.3% | (5.6, 9.6) |
| % all deaths by tertile | 1.7% | (1.3, 2.2) | 1.1% | (0.7, 1.6) | 1.2% | (0.9, 1.6) |

| | | | | | |
|---|---|---|---|---|---|
| Lung Cancer Mortality-rate ratios^{‡} | | | | | |
| Low vs Neutral | ref | 1.73 | (1.14-2.64), | 1.89 | (1.28-2.78), |
| (95% CI), P value | | P | = 0.010 | P | = 0.0009 |

| | | | | |
|---|---|---|---|---|
| Neutral vs High | — | ref | 1.09 | (0.78-1.52), |
| (95% CI), P value | | | P | = 0.62 |

Lung cancer Screening Outcomes

| | Low-CXR vs CT | | Neutral + High-CXR vs CT | |
|---|---|---|---|---|
| Outcomes Low vs Neutral + High | | | | |
| Surgery rates % (95% CI) | 17/39 v 20/34 | | 61/133 v 96/161 | |
| favouring CT over CXR for | 44% v 59% | | 46% v 60% | |
| PRS sub-group | | | | |
| Risk difference-% (95% CI) | −15% | (−38, 7.5) | −14% | (−25, −2.41) |
| P value | P | = 0.21 | P | = 0.019 |
| Lung cancer (LC) lethality | | | | |
| LC lethality in CXR vs CT ^{‡‡} | 18/39 v 12/34 | | 77/133 v 60/161 | |
| % (95% CI) | 46% v 35% | | 58% v 37% | |
| Risk difference-% difference | +11.0 | (−12, 33) | +21% | (9.4, 32) |
| (95% CI) | | | | |
| P value | P | = 0.36 | P | = 0.0004 |
| Odds ratio (OR) of LC lethality in | OR = 1.57 | (0.61, 4.04) | OR = 2.32 | (1.15, 3.70) |
| CXR vs CT (95% CI) | | | | |
| P value | P | = 0.35 | P | = 0.0005 |
| Lung Cancer Deaths averted | | | | |
| Deaths averted favouring CT over | 18/1406 v 12/1405 | | 77/3204 v 60/3174 | |
| CXR | −4.3 | (−3.4, 12) | −5.1 | (−2.0, 12) |
| Absolute difference/1000 | 34% relative reduction | | 21% relative reduction | |
| screened (%) | | | | |
| Odds ratio (OR) of LC deaths | OR = 0.66 | (−0.31, 1.38) | OR = 0.79 | (0.56, 1.10) |
| averted in CXR vs CT (95% CI) | P | = 0.28 | P | = 0.16 |
| P value | | | | |

^{‡}Includes lung cancer deaths identified at post-mortem (N = 180),
^{‡‡} Only lung cancers diagnosed during screening and follow-up (N = 367).
^{β}P value according to PRS group.
(95% CI) = 95% confidence Interval.
P < 0.05 are bold.

TABLE 22
Lung cancer (LC) characteristics according to the lung cancer polygenic
risk score (PRS) score (Low, Neutral, High)

| | Polygenic Risk Score Groups (absolute score range) | | | | | | |
|---|---|---|---|---|---|---|---|
| | Low Score | | Neutral Score | | High Score | | |
| | (−4 to −1) | | 0 | | (1 to 6) | | P value |
| N = 9,191 NHW (% of total cohort) | 2813 | (30.6%) | 2504 | (27.2%) | 3874 | (42.2%) | Total |
| Lung Cancers-N‡ (per 1000) | 78 | (27.7) | 102 | (40.7) | 200 | (51.6) | <0.0001 |
| Demographics | | | | | | | |
| Age-mean (SD) | 64.2 | (5.0) | 63.3 | (5.2) | 64.2 | (5.5) | 0.37 |
| Sex (% Male) | 46 | (59.0%) | 63 | (61.8%) | 104 | (52.0%) | 0.23 |
| Pack Years-mean (SD) | 63.6 | (29.1) | 62.5 | (24.0) | 64.1 | (25.9) | 0.88 |
| Smoking Status (% Current) | 49 | (62.8%) | 58 | (56.9%) | 107 | (53.5%) | 0.37 |
| Self-reported Comorbidity | | | | | | | |
| COPD | 20 | (25.6%) | 36 | (35.3%) | 64 | (32.0%) | 0.38 |
| Pneumonia | 23 | (29.5%) | 34 | (33.3%) | 67 | (33.5%) | 0.80 |
| Heart Dx | 8 | (10.3%) | 20 | (19.6%) | 31 | (15.5%) | 0.23 |
| Diabetes | 5 | (6.4%) | 11 | (10.8%) | 18 | (9.0%) | 0.59 |
| Hypertension | 33 | (42.3%) | 37 | (36.3%) | 74 | (37.0%) | 0.66 |
| Stroke | 1 | (1.3%) | 6 | (5.9%) | 6 | (3.0%) | 0.38 |
| Lung Function | | | | | | | |
| FEV₁/FVC-mean (SD) | 66.57 | (10.76) | 66.10 | (12.0) | 66.39 | (11.8) | 0.96 |
| FEV₁% predicted-mean (SD) | 71.57 | (20.4) | 74.99 | (20.8) | 74.28 | (23.0) | 0.55 |
| GOLD 1-2 | 29 | (37.2%) | 36 | (35.3%) | 74 | (37.0%) | 0.95 |
| GOLD 3-4 | 14 | (18.0%) | 14 | (13.7%) | 28 | (14.0%) | 0.67 |
| Lung Cancer (LC) Characteristics | | | | | | | |
| Lung Cancer Death N (% of LC)‡ | 35/78 | (44.9%) | 54/102 | (52.9%) | 91/200 | (45.5%) | 0.43 |
| Lung Cancer Death N (% of total grp) | 35/2813 | (1.2%) | 54/2504 | (2.2%) | 91/3874 | (2.3%) | 0.0040 |
| Lung Cancer Death during study | 30/73 | (41.1%) | 49/97 | (50.5%) | 88/197 | (44.7%) | 0.45 |
| (% of LC)† | | | | | | | |
| Lung cancer Histology | | | | | | | |
| Small Cell | 12 | (15.4%) | 8 | (7.8%) | 28 | (14.0%) | 0.46 |
| Squamous Cell | 18 | (23.1%) | 26 | (25.5%) | 42 | (21.0%) | |
| Adenocarcinoma/BAC^{Φ} | 29 | (37.2%) | 46 | (45.1%) | 90 | (45.0%) | |
| Non-small/Large Cell | 14 | (18.0%) | 17 | (16.7%) | 36 | (18.0%) | |
| Other/unknown | 5 | (6.4%) | 5 | (4.9%) | 4 | (2.0%) | |
| Lung cancer Stage at Diagnosis N (%) | | | | | | | |
| Stage I-II | 38 | (48.7%) | 42 | (41.2%) | 100 | (50.0%) | 0.35 |
| Stage III-IV | 34 | (43.6%) | 53 | (52.0%) | 93 | (46.5%) | |
| Occult carcinoma/unknown | 6 | (7.7%) | 7 | (6.9%) | 7 | (3.5%) | |
| Surgery-Yes | 37 | (47.4%) | 47 | (46.1%) | 110 | (55.0%) | 0.26 |
| Interval lung Cancer diagnosed | | | | | | | |
| Screening (T0-T2) | 47 | (60%) | 59 | (58%) | 123 | (61%) | 0.83 |
| Follow up (T3-T7) | 31 | (40%) | 43 | (42%) | 77 | (39%) | |
| Cancer detection | | | | | | | |
| Screen-detected | 40 | (51.3%) | 45 | (44.1%) | 104 | (52.0%) | 0.64 |
| Interval/Missed | 7 | (9.0%) | 14 | (13.7%) | 19 | (9.5%) | |
| Follow up | 31 | (39.7%) | 33 | (32.3%) | 77 | (38.5%) | |

‡Total lung cancers including those diagnosed at post-mortem (N = 13),
†Excludes LC diagnosed at post-mortem.
^{Φ}BAC = Bronchioloalveolar Carcinomas.

TABLE 23
Multivariable logistic regression predicting lung cancer death according to known risk
variables (Table 20) including PRS (treated as a continuous variable).

| | Full model | | | Reduced model* | | |
|---|---|---|---|---|---|---|
| | Point | | | Point | | |
| Variable | estimate | 95% CI | P value | estimate | 95% CI | P value |
| Age | 1.07 | 1.04-1.10 | <0.0001 | 1.07 | 1.03-1.10 | <0.0001 |
| Sex (Male vs Female) | 0.98 | 0.67-1.38 | 0.89 | | | |
| Current Smoker | 1.69 | 1.19-2.40 | 0.0031 | 1.69 | 1.19-2.39 | 0.0033 |
| Pack years | 1.01 | 1.01-1.02 | 0.0004 | 1.01 | 1.01-1.02 | 0.0002 |
| Family History of LC | 1.28 | 0.89-1.85 | 0.18 | | | |
| Self-reported COPD | 1.40 | 0.97-2.03 | 0.070 | 1.41 | 0.98-2.02 | 0.07 |
| BMI | 0.95 | 0.91-0.99 | 0.0076 | 0.95 | 0.92-0.99 | 0.0073 |
| High School/Some College | 1.49 | 1.03-2.16 | 0.035 | 1.52 | 1.05-2.19 | 0.027 |
| 12 SNP PRS | 1.18 | 1.06-1.32 | 0.0026 | 1.18 | 1.06-1.32 | 0.0027 |
| COPD (GOLD 1-4)^{#} | 1.45 | 1.03-2.06 | 0.036 | 1.47 | 1.04-2.09 | 0.029 |
| Screening Arm (CXR vs | 1.23 | 0.88-1.71 | 0.22 | | | |
| CT) | | | | | | |

^{#}COPD (GOLD 1-4) = airflow limitation on pre-bronchodilator spirometry,
*Reduced model where variables with p > 0.15 were removed.
LC = lung cancer.
PRS = polygenic risk score.
BMI = body mass index.
CXR = chest x-ray,
CT = computed tomography.

TABLE 24
Algorithm and referencing for the polygenic risk score (PRS)
and clinical score for lung cancer.

| | Clinical score | | | |
|---|---|---|---|---|
| | Criteria | Score | | References |
| Age | ≥65 yo | +4 | | 1-5 |
| Past history of COPD | Yes | +4 | | 1-5 |
| Family history of Lung Cancer | Yes | +3 | | 1-5 |
| Body Mass Index | ≤20 | +4 | | 1-5 |
| | 20-24 | | | |
| | ≥25 | −1 | | |
| Smoking history | 30-39 pack yrs | −2 | | 1-5 |
| | 40-59 pack yrs | | | |
| | ≥60 pack years | +2 | | |
| | Polygenic risk score (PRS) genes | | | |
| | Gene (rs #) | Score | | |
| 1. Nicotinic Acetylcholine | CHRNA | AA | = +1 | 6-20 |
| subunit receptor | [symbol] 16969968) | | | |
| 2 HLA-B-associated transcript 3 | BAT3 (rs1052486) | CC | = +1 | 3, 11, 31 |
| 3 Telomerase reverse transcriptase | TERT (rs402710) | CC | = +1 | 3, 11, 12, 21, 32 |
| 4 Fas Ligand | FasL (rs763110) | TT | = −1 | 1-3, 29, 30 |
| 5 Translesion DNA polymerase | REV1 (rs3087386) | CC | = −1 | 1-3, 28 |
| 6 Matrix metalloproteinase-12 | MMP12 (rs645419) | GG | = −1 | 16, 46, 47 |
| 7 Integrin alpha11 | ITGA11 (rs2306022) | TT/TC | = −1 | 1, 2, 25-27, 31 |
| 8 Interleukin-6 | IL-6 (rs1800797) | GG | = +1 | 52, 53 |
| 9 Iron regulatory binding protein | IREB2 (rs2656069) | CC/CT | = −1 | 11, 16, 35, 36, 45 |
| 10 Cytochrome P450 2A6 | CYP2A6 (rs7937) | TT | = +1 | 13, 16, 45, 49-51 |
| 11 C-reactive protein | CRP (rs2808630) | CC | = −1 | 3, 10, 33 |
| 12 Glycosylation end | AGER (rs2070600) | TT/TC | = +1 | 3, 15, 16, 34, |
| product-specific [symbol] ceptor | | | | 37- [symbol] |

Legend: The risk genotypes assigned to each of the 12 SNP variants were predefined and combined in a previously published algorithm to derive a composite polygenic risk score (PRS) (Table 23). Individuals were scored according to a published algorithm. In 21 individuals, there were 9 or more missing genotypes and these individuals were excluded from the analysis (see FIG. 16 consort diagram). Of the 12 possible risk genotypes, results were available for all 12 SNPs in 8,629 subjects (94%), 11 SNP genotypes in a further 288 subjects (3%) and 10 or more genotypes in 84 subjects (1%). This means over 98% of all subjects had 10 or more SNP genotypes contributing to their PRS. [symbol]
indicates data missing or illegible when filed

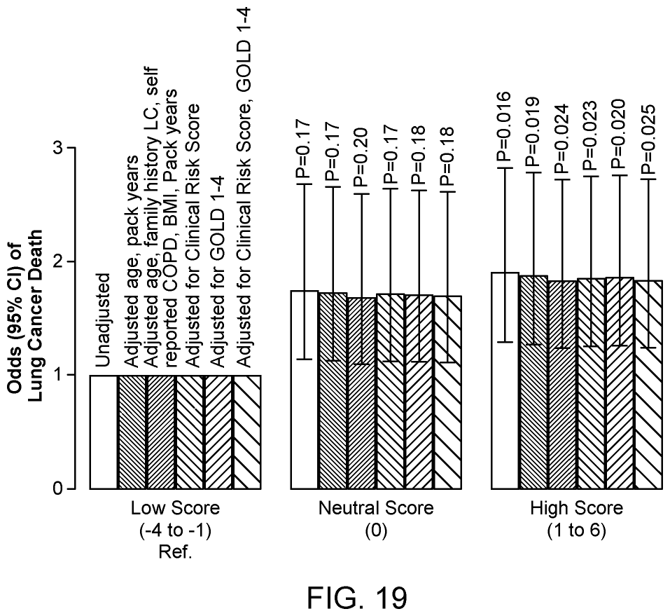

Using area-under-the-curve (AUC) receiver operating characteristics (ROC) analyses to derive a C-statistic the risk of dying of lung cancer according to the PRS alone was found to have AUC=0.568 (95% Confidence Interval (CI)=0.528-0.608, P=0.0009) and according to PRS and clinical score AUC=0.661 (95% CI=0.623-0.698, P<0.0001). Using Odds Ratio Estimates to model dying of lung cancer, it was found that each clinical score of 1 added 0.15 increase to the Odds of lung cancer death and each PRS score of 1 added 0.17 increase indicating comparable weighting in the model. Using the same analysis for risk of developing lung cancer the clinical score alone was found (AUC=0.647, 95% CI 0.620-0.674, P<0.0001) while adding the PRS score increased the AUC (AUC=0.667, 95% CI 0.640-0.693, P=0.018). When this was limited to those in the CT arm, the clinical only AUC (0.640, 95% CI 0.602-0.678) increased when adding the PRS (0.668, 95% CI 0.602-0.705, P=0.011). Using the PLCO_{M2012} model for developing lung cancer in this cohort, it was found that an AUC=0.677 (95% CI 0.651-0.704, P<0.001). The 6-year risk of developing lung cancer based on the gene-based model compared favourably with the PLCO_{M2012} model (FIG. 19).

### Discussion

In this clinical validity study using a subgroup of the NLST, it was found that a lung cancer polygenic risk score (PRS), calculated from 12 independent risk genotypes, correlates with lung cancer mortality. The PRS is based on germ-line mutations and is independent of lung cancer risk factors notably, age, smoking, family history, education, BMI, lung function and pre-existing comorbidity. It was surprising to find that elevated PRS is also significantly and linearly associated with greater lung cancer deaths. This could not be explained by variation in lung cancer stage, histology, detection methods, screening interval or surgical rates. Although there was a weak association with smoking pack years across the PRS groups, due primarily to smoking one extra cigarette per day, this would not explain the findings. Similarly, there was marginally more airflow limitation in those with an elevated PRS but neither of these, nor any other clinical variables examined after extensive adjustment, could account for the persisting effect between PRS and lung cancer mortality in the multivariable model. While it is noted that PRS was associated with lung cancer lethality and lung cancer mortality (but not other causes of death), a significant effect on mortality reduction favouring CT was not shown. Thus, lung cancer PRS appears to provide an independent and specific biomarker of lung cancer death.

There has been considerable interest in assessing high risk smokers eligible for lung cancer screening using clinical risk models. The most validated of these risk models is the PLCO_{M2012} which combines a number of clinical risk variables to derive a 6-year risk for developing lung cancer. However, PLCO_{M2012} also predicts the likelihood of having airflow limitation and COPD. This is important because pre-existing COPD is associated with greater lung cancer risk, more aggressive lung cancer histological subtypes, less surgery, more comorbidity with increasing non-lung cancer deaths and reduced benefit of lung cancer screening. This means those at greatest risk of lung cancer may not achieve the greatest benefits from screening due to an attenuation of this risk-benefit relationship at the highest end of the risk spectrum. As the benefits of lung cancer screening results from a complex interplay between a number of variables, it might be helpful to identify a biomarker of lung cancer lethality as part of the general risk assessment of screening participants. This is because it is recognised there exist considerable biological variability between lung cancers and that this variability will affect outcomes following screening. For example, elderly participants with a high PRS and high clinical risk score with severe COPD may be advised the harms of screening may outweigh the benefits. Similarly, a low PRS with low clinical score and negative CT may defer screening or screen two yearly. Alternatively, an elevated PRS may be used as a motivational tool for screening adherence and smoking cessation. A biomarker of lung cancer biology may be combined with clinical risk variables to improve screening outcomes (participation or adherence) and better individualise the risk versus benefits of screening.

The utility of adding genetic data to clinical risk scores of cancer has been gaining support in the clinical setting of optimising screening for prostate and breast cancer. This study shows that PRS, based on previously validated SNPs, adds to the risk assessment for lung cancer in a lung cancer screening cohort where lung cancer was diagnosed prospectively. Furthermore, it was found that PRS is associated with a small increase in risk of developing lung cancer (improves the AUC when added to the clinical score), it is associated with a 1.7-1.9-fold greater mortality from lung cancer. This latter finding persisted after extensive adjustment for various clinical variables and was also independent of known lung cancer variables such as stage and histology.

It is noted that risk genotypes in the PRS were identified in younger lighter smokers, suggesting potential utility in those recently included in the new USPSTF eligibility criteria. Interestingly, it was found that time to lung cancer diagnosis was shorter in the high PRS group (data not shown). Lastly, the PRS was able to be correlated with who died of their lung cancer rather than just who developed lung cancer in the setting of a retrospective case-control study. A novel approach in this study was the inclusion of genetic factors underlying both lung cancer and COPD. It is noted that the PRS did correlate weakly with smoking intensity and COPD but not severity of airflow limitation. These represent potentially confounding (or mediating) effects that have to date been overlooked when examining the genetic basis of lung cancer.

In summary, in the current study, a polygenic risk score was calculated for lung cancer that appears to correlate with lung cancer lethality in a prospective clinical validity study. PRS may contribute to assessing lung cancer screening participants with the aim to better identify screening participants who might benefit. A gene-based approach to selecting screening participants may have cost-effective benefits by reducing the number needed to screen (NNS) to prevent one lung cancer. This may have considerable importance in targeting younger lighter smokers recently recommended to be included in lung cancer screening. This approach deviates from existing approaches which recommends targeting those at greatest risk (risk-based selection) to lung cancer screening. An outcomes-based approach to lung cancer screening may be of greater utility and that germline mutations represent a very stable, cheap and acceptable bio-assay to incorporate into the optimisation of screening programmes.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1. A method for processing or analyzing a bodily sample in a subject, comprising:
(a) analyzing said bodily sample to yield a first data set comprising at least one genetic polymorphism in said bodily sample, wherein said at least one genetic polymorphism is associated with a first pulmonary or respiratory disease or disorder;
(b) analyzing said bodily sample to yield a second data set comprising at least one genetic polymorphism in said bodily sample, wherein said at least one genetic polymorphism is associated with a second pulmonary or respiratory disease or disorder;
(c) imaging a pulmonary nodule from said subject to determine at least one characteristic associated with said image of said pulmonary nodule;
(d) obtaining data with respect to at least one biomarker of said subject;
(e) computer processing said first data set and said second data set to yield a third data set, and analyzing said third data set in conjunction with said at least one characteristic of said pulmonary nodule of (c) and said at least one biomarker of (d) to determine the risk associated with said pulmonary nodule; and
(f) electronically outputting a report that identifies the risk presented by said pulmonary nodule.

2. The method of claim 1, wherein said pulmonary nodule is detected during a procedure or visit that is related to lung cancer screening.

3. The method of claim 1, wherein said pulmonary nodule is detected during a procedure or visit that is not related to lung cancer screening.

4. (canceled)

5. (canceled)

6. (canceled)

7. (canceled)

8. The method of claim 1, further comprising determining the level of said subject's risk of developing lung cancer, and categorizing said risk as high, intermediate, or low.

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. The method of claim 1, further comprising analyzing at least one of: total number of pulmonary nodule, pulmonary nodule size, pulmonary nodule speculation, pulmonary nodule type, pulmonary nodule location, and pulmonary nodule lobulation.

21. The method of claim 1, further comprising determining a clinical score.

22. The method of claim 1, wherein said first data set or said second data set comprises at least two, at least five, at least ten, or at least fifteen single nucleotide polymorphisms (SNPs) selected from:
(1) a polymorphism at rs1489759 of Hedgehog Interacting Protein (HHIP) gene,
(2) a polymorphism at rs13141641 of Hedgehog Interacting Protein (HHIP) gene,
(3) a polymorphism at rs2202507 of Glycophorin A (GYPA) gene,
(4) a polymorphism at rs7671167 of Family with Sequence Similarity 13 Member A (FAM13A) gene,
(5) a polymorphism at rs754388 of Ras and Rab Interactor 3 (RIN3) gene,
(6) a polymorphism at rs11168048 of 5-Hydroxytryptamine Receptor 4 (HTR4) gene,
(7) a polymorphism at rs58863591 of Deleted in Liver Cancer 1 (DLC1) gene,
(8) a polymorphism at rs1422795 of A Disintegrin and Metalloproteinase 19 (ADAM19) gene,
(9) a polymorphism at rs2306022 of Integrin Alpha 11 (ITGA11) gene,
(10) a polymorphism at rs2808630 of C-Reactive Protein (CRP) gene,
(11) a polymorphism at rs3087386 of DNA Repair Protein (Rev1) gene,
(12) a polymorphism at rs645419 of Matrix Metalloproteinase-12 (MMP12) gene,
(13) a polymorphism at rs763110 of Fas Ligand (FasL) gene,
(14) a polymorphism at rs1052486 of HLA-B-Associated Transcript 3 (BAT3) gene,
(15) a polymorphism at rs1800797 of Interleukin-6 (IL-6) gene,
(16) a polymorphism at rs2070600 of Advanced Glycosylation End Product-Specific Receptor (AGER) gene,
(17) a polymorphism at rs2656069 of Iron Regulatory Protein (IREB) gene,
(18) a polymorphism at rs16969968 of Cholinergic Receptor Nicotinic Alpha 3/5 (CHRNA3/5) gene,
(19) a polymorphism at rs402710 of Telomerase Reverse Transcriptase (TERT) gene, and
(20) a polymorphism at rs7937 of Cytochrome P450 Family 2 Subfamily A Member 6 (CYP2A6) gene.

23. The method of claim 1, wherein said first data set or said second data set comprises any SNP in full linkage disequilibrium to one or more of said SNPs of claim 22.

24. A method for processing or analyzing a bodily sample of a subject, comprising:
(a) analyzing said bodily sample to yield a first data set comprising at least one genetic polymorphism in said bodily sample, wherein said at least one genetic polymorphism is associated with a first pulmonary or respiratory disease or disorder, and a second data set comprising at least one genetic polymorphism in said bodily sample, wherein said at least one genetic polymorphism is associated with a second pulmonary or respiratory disease or disorder;
(b) imaging a pulmonary nodule from said subject to determine at least one characteristic associated with said image of said pulmonary nodule;
(c) obtaining data with respect to at least one biomarker of said subject
(d) computer processing said first data set and said second data set to produce a third data set, and further analyzing said third data set and said at least one characteristic of said pulmonary nodule of (b), to determine the risk associated with said pulmonary nodule to an accuracy of at least about 80%, wherein said accuracy is a percentage of a plurality of independent test samples corresponding to (1) a first plurality of subjects having a pulmonary nodule exhibiting malignancy to (2) a second plurality of subjects having a pulmonary nodule not exhibiting malignancy; and
(e) electronically outputting a report that identifies said risk presented by said pulmonary nodule.

25. (canceled)

26. (canceled)

27. (canceled)

28. (canceled)

29. The method of claim 24, further comprising determining the level of said subject's risk of developing lung cancer, and categorizing said risk as high, intermediate, or low.

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. The method of claim 24, wherein said first data set or said second data set comprises 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, or all 22 single nucleotide polymorphisms (SNPs) selected from:
(1) a polymorphism (e.g., GG genotype) at rs1489759 of human Hedgehog interacting protein (HHIP) gene,
(2) a polymorphism (e.g., CC genotype) at rs13141641 of human Hedgehog interacting protein (HHIP) gene,
(3) a polymorphism (e.g., GG or/and CC genotype) at rs2202507 of glycophorin A (GYPA) gene,
(4) a polymorphism (e.g., CC genotype) at rs7671167 of family with sequence similarity 13 member A (FAM13A) gene,
(5) a polymorphism (e.g., CG or GG genotype) at rs754388 of Ras and Rab interactor 3 (RIN3) gene,
(6) a polymorphism (e.g., CC genotype) at rs11168048 of 5-hydroxytryptamine receptor 4 (HTR4) gene,
(7) a polymorphism (e.g., CT, or TT genotype) at rs58863591 of deleted in liver cancer 1 (DLC1) gene,
(8) a polymorphism (e.g., CC genotype) at rs1422795 in a disintegrin and metalloproteinase 19 (ADAM19) gene,
(9) a polymorphism (e.g., TT or TC genotype) at rs2306022 integrin alpha 11 (ITGA11) gene,
(10) a polymorphism (e.g., CC genotype) at rs2808630 of C-reactive protein (CRP) gene,
(11) a polymorphism (e.g., GG genotype) at rs3087386 of DNA repair protein (Rev1) gene,
(12) a polymorphism (e.g., GG genotype) at rs645419 of matrix metalloproteinase-12 (MMP12) gene,
(13) a polymorphism (e.g., TT genotype) at rs763110 of Fas ligand (FasL) gene,
(14) a polymorphism (e.g., GG genotype) at rs1052486 of HLA-B-associated transcript 3 (BAT3) gene,
(15) a polymorphism (e.g., GG genotype) at rs1800797 of interleukin-6 (IL-6) gene,
(16) a polymorphism (e.g., TT or TC genotype) at rs2070600 of advanced glycosylation end product-specific receptor (AGER) gene,
(17) a polymorphism (e.g., CC or CT genotype) at rs2656069 of iron regulatory protein (IREB) gene,
(18) a polymorphism (e.g., AA genotype) at rs16969968 of cholinergic receptor nicotinic alpha 3/5 (CHRNA3/5) gene,
(19) a polymorphism (e.g., CC genotype) at rs402710 of telomerase reverse transcriptase (TERT) gene, and
(20) a polymorphism (e.g., TT genotype) at rs7937 of cytochrome P450 family 2 subfamily A member 6 (CYP2A6) gene.

64. The method of claim 24, wherein said first data set or said second data set comprises one or more single nucleotide polymorphisms (SNPs) selected from (i)-(vii) (e.g., from (iii)-(vii)):
(i) at least one chronic obstructive pulmonary disease (COPD)-protective SNP;
(ii) at least one COPD-susceptible SNP;
(iii) at least one lung cancer-protective SNP;
(iv) at least one lung cancer-susceptible SNP;
(v) at least one lung cancer-susceptible, COPD-protective SNP;
(vi) at least one lung cancer and COPD dual protective SNP; and
(vii) at least one lung cancer and COPD dual susceptible SNP.

65. (canceled)

66. (canceled)

67. (canceled)

68. (canceled)

69. (canceled)

70. (canceled)

71. (canceled)

72. The method of claim 24, wherein, in (c), said subject is determined to be predisposed to develop or die of lung cancer within a period of time (e.g., about 5 years).

73. (canceled)

74. (canceled)

75. (canceled)

76. (canceled)

77. (canceled)

78. (canceled)

79. (canceled)

80. (canceled)

81. The method of claim 24, wherein said bodily sample is selected from the group consisting of: a blood sample, a serum sample, a plasma sample, a saliva sample, a stool sample, a sputum sample, a urine sample, a semen sample, a transvaginal fluid sample, a cerebrospinal fluid sample, a sweat sample, a cell sample, and a tissue sample.

82. (canceled)

83. (canceled)

84. (canceled)

85. (canceled)

86. (canceled)

87. (canceled)

88. The method of claim 24, wherein said at least one biomarker comprises analysis of proteins, antibodies, or nucleic acids (e.g., liquid or tumor DNA, tumor RNA) obtained from said subject.

89. (canceled)

90. (canceled)

91. (canceled)

92. (canceled)

93. The method of claim 24, wherein said an improved clinical outcome is lower surgery rate for benign nodules.

94. (canceled)

95. (canceled)

96. (canceled)

97. A method for processing or analyzing a bodily sample of a subject, comprising:
(a) analyzing said bodily sample to yield a data set comprising at least one genetic polymorphism in said bodily sample, wherein said at least one genetic polymorphism is associated with a pulmonary or respiratory disease or disorder;
(b) computer processing said data set from (a) and a clinical health parameter of said subject to determine a risk-value of said pulmonary or respiratory disease or disorder in said subject; and
(c) electronically outputting a report that identifies said risk-value of said pulmonary or respiratory disease or disorder in said subject determined in (b).

98. (canceled)

99. (canceled)

100. (canceled)

101. (canceled)

102. (canceled)

103. (canceled)

104. (canceled)

105. (canceled)

106. (canceled)

107. (canceled)

108. (canceled)

109. (canceled)

110. (canceled)

111. (canceled)

112. (canceled)

113. (canceled)

114. (canceled)

115. (canceled)

116. (canceled)

117. (canceled)

118. (canceled)

119. (canceled)

120. (canceled)

121. (canceled)

122. (canceled)

123. (canceled)

124. (canceled)

125. (canceled)

126. The method of claim 97, wherein a risk-value indicates a presence, a reduced risk, or an elevated risk of said pulmonary or respiratory disease or disorder.

127. (canceled)

128. (canceled)

129. (canceled)

130. (canceled)

131. (canceled)

132. (canceled)

133. (canceled)

134. (canceled)

135. (canceled)

136. (canceled)

137. (canceled)

138. (canceled)

139. (canceled)

140. (canceled)

141. (canceled)

142. (canceled)

143. (canceled)

144. The method of claim 97, wherein said subject has or had an exposure to smoking (e.g., active or passive smoking) (e.g., tobacco smoking, such as cigarette smoking, pipe smoking, cigar smoking, use of chewing tobacco, or a combination thereof).

145. (canceled)

146. The method of claim 97, wherein said bodily sample is selected from the group consisting of: a blood sample, a serum sample, a plasma sample, a saliva sample, a buccal sample, a stool sample, a sputum sample, a urine sample, a semen sample, a transvaginal fluid sample, a cerebrospinal fluid sample, a sweat sample, a cell sample, and a tissue sample.

147. (canceled)

148. (canceled)

149. (canceled)

150. (canceled)

151. (canceled)

152. (canceled)

153. (canceled)

154. (canceled)

155. (canceled)

156. (canceled)

157. (canceled)
